(19) United States
(12) Patent Application Publication
(10) Pub. No.: US 2026/0308676 A1
(43) Pub. Date: Oct. 8, 2026

(54) METHOD AND PRODUCTS FOR CHARACTERIZING A POLYNUCLEOTIDE USING A NANOPORE

(71) Applicant: Oxford Nanopore Technologies PLC, Oxford (GB)

(72) Inventors: Lukasz Ciszewski, Oxford (GB); Sebastien James, Oxford (GB); Graham James Hall, Oxford (GB); Olle Alfred Nordesjö, Oxford (GB)

(73) Assignee: Oxford Nanopore Technologies PLC, Oxford (GB)

(21) Appl. No.: 19/484,909

(22) PCT Filed: May 17, 2024
(86) PCT No.: PCT/GB2024/051301

§ 371 Date: Nov. 17, 2025

(30) Foreign Application Priority Data
May 18, 2023 (GB) 2307486.7

(51) Int. Cl. C12Q 1/6869; G01N 33/487

(52) U.S. Cl. CPC C12Q 1/6869; G01N 33/48721

(57) ABSTRACT

Provided herein are methods of characterising a polynucleotide during the translocation of the polynucleotide through a nanopore. Also provided herein are methods of operating a nanopore array. Further provided herein are kits and apparatuses for carrying out such methods.

### RELATED APPLICATIONS

This application is a national stage filing under 35 U.S.C. § 371 of international application number PCT/GB2024/051301, filed May 17, 2024, which claims the benefit of United Kingdom application number GB 2307486.7, filed May 18, 2023, each of which is herein incorporated by reference in its entirety.

### REFERENCE TO AN ELECTRONIC SEQUENCE LISTING

The contents of the electronic sequence listing (0036670177US00-SEQ-KZM.xml; Size: 13,033 bytes; and Date of Creation: Oct. 17, 2025) are herein incorporated by reference in their entirety.

### FIELD OF THE INVENTION

The present disclosure provides methods of characterising a polynucleotide as it translocates through a nanopore. The disclosure also provides methods of operating a nanopore array. The disclosure also provides novel kits and apparatuses for use in the methods of the disclosure.

### BACKGROUND

The characterisation of biological molecules is of increasing importance in biomedical and biotechnological applications. For example, sequencing of nucleic acids allows the study of genomes and the proteins they encode and, for example, allows correlation between nucleic acid mutations and observable phenomena such as disease indications. Nucleic acid sequencing can be used in evolutionary biology to study the relationship between organisms. Metagenomics involves identifying organisms present in samples, for example microbes in a microbiome, with nucleic acid sequencing allowing the identification of such organisms.

Nanopore sensing of polynucleotide analytes can reveal the identity and perform single molecule counting of the sensed analytes in real time, but can also provide information on their composition such as their nucleotide sequence, as well as the presence of characteristics such as base modifications, oxidation, reduction, decarboxylation, deamination and more. Nanopore sensing has the potential to allow rapid and cheap polynucleotide sequencing, providing single molecule sequence reads of polynucleotides of tens to tens of thousands bases length. Furthermore, the potential to create very long reads has a number of benefits over traditional next-generation sequencing technologies that produce short reads (typically <300 bp). For example, in the context of whole genome sequencing, the longer reads produced by nanopore sensing allows easier assembly of DNA fragments, improved characterisation of repetitive genomic regions and identification of large structural variations.

Two of the essential components of polymer characterization using nanopore sensing are (1) the control of polymer movement through the pore and (2) the discrimination of the component building blocks as the polymer is moved through the pore. During nanopore sensing of analytes such as polynucleotides, it is important to control the movement of the polynucleotide with respect to the pore. Uncontrolled movement can prevent or impede accurate characterisation of the polynucleotides. For example, accurately distinguishing each nucleotide in a homopolymeric polynucleotide is problematic when the movement of the polynucleotide with respect to the pore is not controlled.

In order to maximise the utility of characterisation methods, it is further necessary to maximise the efficiency of the methods used. In part this may involve efficiently controlling the movement of the polynucleotide with respect to the detector used to characterise the polynucleotides, such as a nanopore, for example by controlling the movement of a polynucleotide with respect to a detector such as a nanopore by using a motor protein to control the movement of the polynucleotide. However, a further aspect involves reducing unproductive characterisation of species which may be present in a sample but which are not the desired analyte. There is a need for such “unproductive characterisation” to be reduced.

A further issue that may be encountered during the translocation of a polynucleotide through a detector such as a nanopore is known as blocking (e.g. pore blocking). Without being bound by theory, it is believed that pore blocking may arise due to a variety of reasons, for example due to the formation of secondary structure by the polynucleotide on the side of the nanopore that is first contacted with the polynucleotide, the binding of co-factors or proteins to the polynucleotide on the side of the nanopore that is first contacted with the polynucleotide, or simply the speed at which the polynucleotide is translocated. Particularly for long analytes, pore blocking is a key limiting factor on the amount of information that may be obtained from a nanopore array.

One known approach to address pore blocking is to seek to translocate the polynucleotide back through the nanopore (or other detector used) such that the direction of movement of the polynucleotide with respect to the pore is reversed. This may allow the problematic polynucleotide to fully eject from the pore and allow for a new polynucleotide to translocate through the pore.

However, problems persist. In particular, attempts to unblock pores are often unsuccessful. Over time, pore blocking will reduce the efficiency in which a detector array (e.g. a nanopore array) can be used, and thus limit the amount of information which can be obtained. Blocking also typically effectively limits the functional lifetime of detector arrays (e.g. nanopore arrays). Therefore, there remains a need for ways of reducing blocking, particularly in nanopore systems.

For these and other reasons, there is a need for new and/or improved methods of characterising polynucleotides.

### SUMMARY OF THE INVENTION

As described in more detail herein, and without being bound by theory, the inventors believe that the formation of cation-mediated secondary structure may contribute to blocking of detector arrays (e.g. nanopore arrays). The inventors have now found that conditions which decrease formation of secondary structure can be beneficially used to improve the quality of data that is obtained from a nanopore array.

Accordingly, in one aspect, the disclosure relates to methods of characterising a polynucleotide. The polynucleotide is contacted with a nanopore, which has a first opening and a second opening. A portion of the polynucleotide is translocated through the nanopore in the direction from the first opening to the second opening. The portion of the polynucleotide that has translocated through the nanopore is then brought into contact with conditions that are present on the side of nanopore having the second opening. The conditions decrease the formation of cation-mediated secondary structure by the portion of the polynucleotide that has translocated through the pore. The portion of the polynucleotide is subsequently translocated through the nanopore in the direction from the second opening to the first opening. One or more measurements that are characteristic of the polynucleotide are taken during the translocation of the polynucleotide through the nanopore to thereby characterise the polynucleotide. The one or more measurements may be taken when the polynucleotide translocates through the nanopore in the direction from the first opening to the second opening and/or when the polynucleotide translocates through the nanopore in the direction from the second opening to the first opening.

In another aspect, the disclosure relates to methods of operating a nanopore array. Each nanopore in the array has a first opening and a second opening. A plurality of polynucleotides are contacted with the array. The conditions under which the plurality of polynucleotides are contacted with the array lead to a portion of each polynucleotide translocating through the nanopores in the array in the direction from the first opening to the second opening, i.e. a portion of a first polynucleotide is translocated through a first nanopore in the direction from the first opening to the second opening, a portion of a second polynucleotide is translocated through a second nanopore in the direction from the first opening to the second opening, and so forth. The translocated portion of the polynucleotides that have translocated through the nanopores in the array are brought into contact with conditions that are present on the side of nanopores having the second opening. The conditions decrease the formation of cation-mediated secondary structure by the portion of the polynucleotides that have translocated through the pore. A desired characteristic of each polynucleotide that has been translocated is determined, for example the presence or absence of said characteristic. Polynucleotides which do not have the desired characteristic are ejected from the nanopore by translocating the portion of said polynucleotides through the nanopore in the direction from the second opening to the first opening.

Accordingly, provided herein is a method of characterising a polynucleotide, the method comprising:
(a) contacting the polynucleotide with a nanopore having a first opening and a second opening;
(b) translocating a portion of the polynucleotide through the nanopore in a first direction with respect to the nanopore, wherein the first direction is from the first opening to the second opening;
(c) contacting the translocated portion of the polynucleotide with one or more conditions which decrease formation of cation-mediated secondary structure;
(d) translocating the polynucleotide in a second direction with respect to the nanopore, wherein the second direction is from the second opening to the first opening; and
(e) taking one or more measurements characteristic of the polynucleotide during the translocation of the polynucleotide through the nanopore, thereby characterising the polynucleotide.

In some embodiments, if the measurements characteristic of the polynucleotide are indicative of one or more desired characteristics of the polynucleotide, the method comprises translocating the entire polynucleotide through the nanopore and taking one or more measurements during said translocation.

In some embodiments, the method comprises repeating steps (b) to (d) multiple times.

In some embodiments, if the measurements characteristic of the polynucleotide are indicative of one or more undesired characteristics of the polynucleotide, the method comprises ejecting the polynucleotide from the first opening of the nanopore. In some embodiments, the one or more undesired characteristics include the inability of the polynucleotide to fully translocate through the nanopore in the first direction with respect to the nanopore.

In some embodiments, the method comprises characterising a plurality of polynucleotides using a plurality of nanopores. In some embodiments, the plurality of nanopores are present in a nanopore array.

Also provided herein is a method of operating a nanopore array, wherein each nanopore in the array has a first opening and a second opening, the method comprising:
(a) contacting the array with a plurality of polynucleotides under conditions such that a portion of each polynucleotide translocates through the array in a first direction with respect to the nanopores in the array, wherein the first direction is from the first opening to the second opening;
(b) contacting the translocated portion of the polynucleotides with one or more conditions which decrease formation of cation-mediated secondary structure;
(c) determining whether each polynucleotide translocated in step (b) has a desired characteristic; and
(d) ejecting polynucleotides which do not have the desired characteristic by translocating the polynucleotide in a second direction with respect to the nanopores in the array, wherein the second direction is from the second opening to the first opening.

In some embodiments, the method comprises taking one or more measurements characteristic of the polynucleotides in the plurality of polynucleotides during the translocation of the portion of each polynucleotide through the nanopore, thereby characterising the polynucleotides.

In some embodiments of the above methods, the method is performed at a temperature lower than the melting temperature of the cation-mediated secondary structure.

In some embodiments of the above methods, the cation-mediated secondary structure is a guanine tetrad or a G-quadruplex, preferably a G-quadruplex.

In some embodiments of the above methods, the one or more conditions comprise a salt that disrupts the stability of the cation-mediated secondary structure. In some embodiments of the above methods, the one or more conditions comprise a metal salt that disrupts the stability of the cation-mediated secondary structure. In some embodiments, the metal salt comprises a cation selected from the group consisting of Rb⁺, Na⁺, Cs⁺, Li⁺, Mg²⁺ and Ca²⁺. In some embodiments, the metal salt comprise comprises a Rb⁺ cation. In some embodiments, the metal salt comprises (i) rubidium ferricyanide and/or rubidium ferrocyanide, and/or (ii) rubidium chloride. In some embodiments, the metal salt comprises a Na⁺ cation. In some embodiments, the metal salt comprises sodium ferricyanide and/or sodium ferrocyanide. In some embodiments, the salt comprises an ammonium cation (NH₄⁺). In some embodiments of the above methods, the conditions are substantially free of K⁺.

In some embodiments of the above methods, the polynucleotide, or each polynucleotide in the plurality of the polynucleotides, has a length of 100 nucleotides or more. In some embodiments of the above methods, the polynucleotide has a length of 5000 nucleotides or more, or the polynucleotides in the plurality of polynucleotides have an average length of 5000 nucleotides or more.

In some embodiments of the above methods, the method comprises controlling the movement of the or each polynucleotide with respect to the nanopore in the first direction using a polynucleotide-handling protein. In some embodiments, the polynucleotide-handling protein is or comprises a helicase, translocase or helicase-nuclease complex.

In some embodiments of the above methods, the nanopore is a protein pore, a solid state pore or a DNA origami pore, preferably wherein the nanopore is a transmembrane protein pore.

In some embodiments of the above methods, the one or more characteristics comprises (i) the length of the polynucleotide, (ii) the identity of the polynucleotide, (iii) the sequence of the polynucleotide, (iv) the secondary structure of the polynucleotide and (v) whether or not the polynucleotide is modified.

In some embodiments of the above methods that involve ejecting the polynucleotide from the nanopore, ejecting the polynucleotide from the nanopore comprises applying a force across the nanopore. In some embodiments, the force comprises a voltage potential applied across the nanopore.

In some embodiments of the above methods, the method comprises applying a voltage potential across the nanopore via one or more platinum electrodes.

Also provided herein is a kit for characterising a polynucleotide, comprising (a) a nanopore in a membrane; and (b) a solution comprising one or more metal salts selected from the group consisting of one or more rubidium salts, sodium ferricyanide and sodium ferrocyanide. In some embodiments, the one or more metal salts are selected from the group consisting of (i) rubidium ferricyanide and/or rubidium ferrocyanide, (ii) rubidium chloride, and/or (iii) sodium ferricyanide and sodium ferrocyanide.

Further provided herein is an apparatus for characterising a polynucleotide, comprising (a) an array of nanopores in a membrane; and (b) a solution comprising one or more metal salts in contact with the membrane, wherein the one or more metal salts are selected from the group consisting of one or more rubidium salts, sodium ferricyanide and sodium ferrocyanide. In some embodiments, the one or more metal salts are selected from the group consisting of (i) rubidium ferricyanide and/or rubidium ferrocyanide, (ii) rubidium chloride, and (iii) sodium ferricyanide and sodium ferrocyanide. In some embodiments, the array is an array of individually addressable elements, wherein each independently addressable element of the array comprises a cis and a trans chamber connected by an electrical communication means, wherein said cis and trans chambers are separated by the membrane, and the membrane of each independently addressable element comprises a single nanopore. In some embodiments, the cis and the trans chamber each comprise the solution. In some embodiments, the trans chamber comprises the solution, and the cis chamber comprises a different solution comprising one or more metal salts. In some embodiments, the apparatus comprises a polynucleotide handling enzyme on the cis side of the membrane. In some embodiments, the independently addressable elements of the array share a common cis chamber. In some embodiments, each independently addressable element has a trans chamber separate from the trans chambers of other independently addressable elements of the array. In some embodiments, the apparatus comprises a voltage source connected to electrodes, wherein the voltage source and electrodes are configured to apply an electric field between the cis and the trans chambers. In some embodiments, the apparatus comprises a digital logic circuit associated with the nanopores of the independently addressable elements of the array of independently addressable elements.

Also provided herein is use of one or more metal salts for reducing cation-mediated secondary structure in a polynucleotide in a nanopore apparatus or during a method of characterising a polynucleotide using a nanopore, wherein the one or more metal salts are selected from the group consisting of one or more rubidium salts, sodium ferricyanide and sodium ferrocyanide. In some embodiments, the one or more metal salts are selected from the group consisting of (i) rubidium ferricyanide and/or rubidium ferrocyanide, (ii) rubidium chloride, and (iii) sodium ferricyanide and sodium ferrocyanide.

### BRIEF DESCRIPTION OF THE FIGURES

FIG. 1. Non-limiting schematic showing the translocation of a polynucleotide from a first side (e.g. the cis side) of a nanopore to a second side (e.g. the trans side) of a detector such as a nanopore. In both schematics, secondary structure is formed by the polynucleotide on the trans side of the nanopore. In the top schematic, no issue is encountered when the polynucleotide translocates from the cis to the trans side of the pore, and further polynucleotides may be captured by the nanopore. In the bottom schematic, an issue is encountered that prevents the polynucleotide from fully translocating from the cis to the trans side of the nanopore, resulting in pore blocking. Due to the formation of secondary structure by the polynucleotide on the trans side on the nanopore, it is not possible to eject the polynucleotide on the cis side of the membrane. The pore cannot capture further polynucleotides.

FIG. 2. Estimated output (MB) over time normalised to 1500 nanopores. Y-axis indicates the cumulative sum (y-axis; MB) of data collected from a sample comprising human genomic DNA 30 kb in length over time. Control refers to a buffer comprising a potassium salt on the cis and trans side of the nanopores. Na refers to a buffer comprising a sodium salt on the cis and trans side of the nanopores.

FIG. 3. Estimated output (MB) over time normalised by the number of starting channels. Y-axis indicates the cumulative sum (y-axis; MB) of data collected from a sample comprising a polynucleotide comprising a sequence known to form a G-quadruplex over time. Control refers to a buffer comprising a potassium salt on the cis and trans side of the nanopores. Na and sodium_cistrans refer to a buffer comprising a sodium salt on the cis and trans side of the nanopores. Sodium_cis refers to a buffer comprising a potassium salt on the cis side of the nanopores and a sodium salt on the trans side of the nanopores.

FIG. 4. Melting temperature of a G-quadruplex in the presence of different salts, as determined by fluorescence spectroscopy. The 3′ and 5′ ends of a polynucleotide comprising a G-quadruplex structure were attached to a quencher and a fluorophore such that the fluorescence was quenched when the G-quadruplex was formed around the cation. Upon denaturing of the G-quadruplex, the quenching of the fluorophore was reduced.

FIG. 5. Graph showing normalised fluorescence [-] vs temperature [° C.] for the same G-quadruplex-forming polynucleotide in different ionic solutions (potassium, sodium, and rubidium). Cation type has a large effect on G-quadruplex stability. From left to right, the curves depicted are: Rb⁺, Na⁺ (150 mM), Na⁺ (210 mM), K⁺.

FIG. 6. Output over time for the same 30 kb human genomic DNA sample for three different cation types, as shown by total sequencing output (Gb). Increases in sequencing output for (i) sodium compared to potassium, and (ii) rubidium compared to potassium, are (i) +37% and (ii) +56%, respectively.

FIG. 7. A: Sequencing Output over time comparing rubidium salt flow cells to control potassium salt flow cells. Potassium salt flow cells only were washed and reloaded at 22 hours. Total sequencing outputs remain similar. B: Channel loss over time as a percentage of starting pores. Each line represents one flow cell. Recovery of pores at 22 hours for the potassium flow cells following wash and reload can be seen. The rate of channel loss for the rubidium salt flow cells is less than that for the potassium salt flow cells. This leads to better maintained throughput over time, leading to similar levels of output as in FIG. 7A.

FIG. 8. A: Output over time for the same ULK114 human genomic DNA sample with potassium salts and rubidium salts each with two nuclease washes and reloads at approximately 24 hours and 48 hours. Increase in sequencing output is +46% for the rubidium salts. B: N50 of the ULK114 human genomic sample for both flow cell types, showing that both had read length N50s above 100 kilobases.

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein.

The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment.

It should be appreciated that “embodiments” of the disclosure can be specifically combined together unless the context indicates otherwise. The specific combinations of all disclosed embodiments (unless implied otherwise by the context) are further disclosed embodiments of the claimed invention.

In addition, as used in this specification and the appended claims, the singular forms “a”, “an”, and “the” include plural referents unless the content clearly dictates otherwise. Thus, for example, reference to “a polynucleotide” includes two or more polynucleotides, reference to “a polynucleotide-handling enzyme” includes two or more such polynucleotide-handling enzymes, reference to “a nanopore” includes two or more nanopores and the like.

It is to be understood that whilst the methods, uses and products of the invention have been described with respect to a nanopore, the methods, uses and product of the invention equally relate to the use of a detector in place of a nanopore. The methods, uses and products provided herein are amenable to detectors including (i) a zero-mode waveguide, (ii) a field-effect transistor, optionally a nanowire field-effect transistor; (iii) an AFM tip; (iv) a nanotube, optionally a carbon nanotube and (v) a nanopore. The disclosed methods, uses and products are particularly amenable to applications in which a polynucleotide is moved through a detector or through a structure containing a detector, e.g. a well in a detector chip.

All publications, patents and patent applications cited herein, whether supra or infra, are hereby incorporated by reference in their entirety.

### Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016), for definitions and terms of the art. The definitions provided herein should not be construed to have a scope less than understood by a person of ordinary skill in the art.

“About” as used herein when referring to a measurable value such as an amount, a temporal duration, and the like, is meant to encompass variations of +20% or +10%, more preferably +5%, even more preferably +1%, and still more preferably +0.1% from the specified value, as such variations are appropriate to perform the disclosed methods.

“Nucleotide sequence” or “nucleic acid molecule(s)” as used herein refers to a polymeric form of nucleotides of any length, either ribonucleotides or deoxyribonucleotides. This term refers only to the primary structure of the molecule. Thus, this term includes double- and single-stranded DNA, and RNA. The term “nucleic acid” as used herein, is a single or double stranded covalently-linked sequence of nucleotides in which the 3′ and 5′ ends on each nucleotide are joined by phosphodiester bonds. The polynucleotide may be made up of deoxyribonucleotide bases or ribonucleotide bases. Nucleic acids may be manufactured synthetically in vitro or isolated from natural sources. Nucleic acids may further include modified DNA or RNA, for example DNA or RNA that has been methylated, or RNA that has been subject to post-translational modification, for example 5′-capping with 7-methylguanosine, 3′-processing such as cleavage and polyadenylation, and splicing. Nucleic acids may also include synthetic nucleic acids (XNA), such as hexitol nucleic acid (HNA), cyclohexene nucleic acid (CeNA), threose nucleic acid (TNA), glycerol nucleic acid (GNA), locked nucleic acid (LNA) and peptide nucleic acid (PNA). Sizes of nucleic acids, also referred to herein as “polynucleotides” are typically expressed as the number of base pairs (bp) for double stranded polynucleotides, or in the case of single stranded polynucleotides as the number of nucleotides (nt). One thousand bp or nt equal a kilobase (kb). Polynucleotides of less than around 40 nucleotides in length are typically called “oligonucleotides” and may comprise primers for use in manipulation of DNA such as via polymerase chain reaction (PCR).

The term “amino acid” in the context of the present disclosure is used in its broadest sense and is meant to include organic compounds containing amine (NH₂) and carboxyl (COOH) functional groups, along with a side chain (e.g., a R group) specific to each amino acid. In some embodiments, the amino acids refer to naturally occurring L α-amino acids or residues. The commonly used one and three letter abbreviations for naturally occurring amino acids are used herein: A=Ala; C-Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term “amino acid” further includes D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as B-amino acids. For example, analogues or mimetics of phenylalanine or proline, which allow the same conformational restriction of the peptide compounds as do natural Phe or Pro, are included within the definition of amino acid. Such analogues and mimetics are referred to herein as “functional equivalents” of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

The terms “leader” and “leader sequence” are used interchangeably herein.

The terms “polypeptide”, and “peptide” are interchangeably used herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. Polypeptides can also undergo maturation or post-translational modification processes that may include, but are not limited to: glycosylation, proteolytic cleavage, lipidization, signal peptide cleavage, propeptide cleavage, phosphorylation, and such like. A peptide can be made using recombinant techniques, e.g., through the expression of a recombinant or synthetic polynucleotide. A recombinantly produced peptide it typically substantially free of culture medium, e.g., culture medium represents less than about 20%, more preferably less than about 10%, and most preferably less than about 5% of the volume of the protein preparation.

The term “protein” is used to describe a folded polypeptide having a secondary or tertiary structure. The protein may be composed of a single polypeptide, or may comprise multiple polypeptides that are assembled to form a multimer. The multimer may be a homooligomer, or a heterooligmer. The protein may be a naturally occurring, or wild type protein, or a modified, or non-naturally, occurring protein. The protein may, for example, differ from a wild type protein by the addition, substitution or deletion of one or more amino acids.

A “variant” of a protein encompass peptides, oligopeptides, polypeptides, proteins and enzymes having amino acid substitutions, deletions and/or insertions relative to the unmodified or wild-type protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

For all aspects and embodiments of the present invention, a “variant” has at least 50%, 60%, 70%, 80%, 90%, 95% or 99% complete sequence identity to the amino acid sequence of the corresponding wild-type protein. Sequence identity can also be to a fragment or portion of the full-length polynucleotide or polypeptide. Hence, a sequence may have only 50% overall sequence identity with a full-length reference sequence, but a sequence of a particular region, domain or subunit could share 80%, 90%, or as much as 99% sequence identity with the reference sequence.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant” or “variant” refers to a gene or gene product that displays modifications in sequence (e.g., substitutions, truncations, or insertions), post-translational modifications and/or functional properties (e.g., altered characteristics) when compared to the wild-type gene or gene product. It is noted that naturally occurring mutants can be isolated; these are identified by the fact that they have altered characteristics when compared to the wild-type gene or gene product. Methods for introducing or substituting naturally-occurring amino acids are well known in the art. For instance, methionine (M) may be substituted with arginine (R) by replacing the codon for methionine (ATG) with a codon for arginine (CGT) at the relevant position in a polynucleotide encoding the mutant monomer. Methods for introducing or substituting non-naturally-occurring amino acids are also well known in the art. For instance, non-naturally-occurring amino acids may be introduced by including synthetic aminoacyl-tRNAs in the IVTT system used to express the mutant monomer. Alternatively, they may be introduced by expressing the mutant monomer in E. coli that are auxotrophic for specific amino acids in the presence of synthetic (i.e. non-naturally-occurring) analogues of those specific amino acids. They may also be produced by naked ligation if the mutant monomer is produced using partial peptide synthesis. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 1 below. Where amino acids have similar polarity, this can also be determined by reference to the hydropathy scale for amino acid side chains in Table 2.

TABLE 1
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, charged (+) | Thr | polar, hydrophilic, neutral |
| Ile | aliphatic, hydrophobic, neutral | Val | aliphatic, hydrophobic, neutral |
| Lys | polar, hydrophilic, charged (+) | Trp | aromatic, hydrophobic, neutral |
| Leu | aliphatic, hydrophobic, neutral | Tyr | aromatic, polar, hydrophobic |

TABLE 2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

A mutant or modified protein, monomer or peptide can also be chemically modified in any way and at any site. A mutant or modified monomer or peptide is preferably chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The mutant of modified protein, monomer or peptide may be chemically modified by the attachment of any molecule. For instance, the mutant of modified protein, monomer or peptide may be chemically modified by attachment of a dye or a fluorophore.

### Disclosed Methods

In one aspect, the disclosure relates to a method of characterising a polynucleotide as it translocates through a nanopore having a first opening and a second opening. A portion of the polynucleotide translocates in a first direction with respect to the nanopore, wherein the first direction is from the first opening to the second opening. The portion of the polynucleotide translocates through the nanopore in a second direction with respect to the nanopore, wherein the second direction is from the second opening to the first opening.

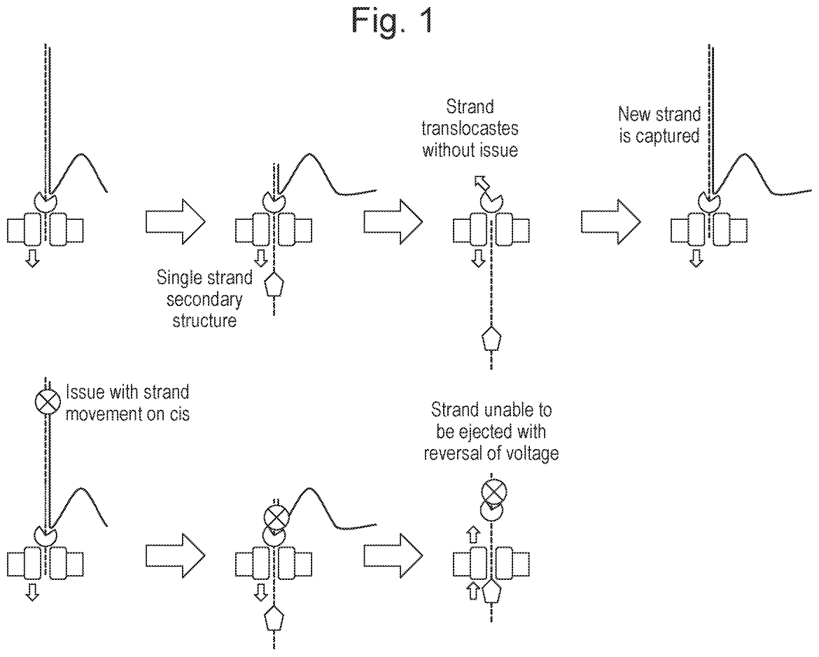

The portion of the polynucleotide that translocates through the nanopore is contacted with one or more conditions which decrease formation of cation-mediated secondary structure. Without being bound by theory, the inventors believe that the formation of secondary structure by the portion of the polynucleotide that has translocated through the pore may prevent the movement of the polynucleotide in the second direction, such that if the pore becomes blocked by a polynucleotide, thus preventing translocation of the polynucleotide from the first opening to the second opening, ejecting the polynucleotide by reversing the direction of movement of the polynucleotide through the pore is also impeded. This is described in FIG. 1.

Without being bound by theory, the inventors consider that this phenomenon could account for a substantial portion of the observed decrease in nanopore array lifetimes and limited nanopore efficiency, particularly when addressing long nanopores. More specifically, (without being bound by theory) the inventors consider that cation-mediated secondary structure may be responsible for as much as 30-40% of instances where a pore becomes “permanently blocked”, i.e. is unable to be unblocked by reversing the direction of movement of a polynucleotide through a pore.

The inventors have now found that by contacting the portion of the polynucleotide that has translocated through the pore with conditions which decrease formation of cation-mediated secondary structure by the polynucleotide, a greater proportion of pore blockages are able to be unblocked. The functional lifetime of nanopore arrays can thus be increased and the characterisation data obtained when a nanopore array is used to characterise a sample of polynucleotides can be improved.

Accordingly, in one aspect is provided herein a method of characterising a target polynucleotide, comprising
contacting the polynucleotide with a nanopore having a first opening and a second opening;
translocating a portion of the polynucleotide through the nanopore in a first direction with respect to the nanopore, wherein the first direction is from the first opening to the second opening;
contacting the translocated portion of the polynucleotide with one or more conditions which decrease formation of cation-mediated secondary structure;
translocating the polynucleotide in a second direction with respect to the nanopore, wherein the second direction is from the second opening to the first opening; and
taking one or more measurements characteristic of the polynucleotide during the translocation of the polynucleotide through the nanopore, thereby characterising the polynucleotide.

In some embodiments, if the measurements characteristic of the polynucleotide are indicative of one or more desired characteristics of the polynucleotide, the method comprises translocating the entire polynucleotide through the nanopore and taking one or more measurements during said translocation. In some embodiments, if the measurements characteristic of the polynucleotide are indicative of one or more undesired characteristics of the polynucleotide, the method comprises ejecting the polynucleotide from the first opening of the nanopore. This enables the method to be used in an “adaptive sampling” approach to characterisation of a polynucleotide. If the measurement of a portion of the polynucleotide is indicative of a desired characteristic, e.g. a genomic region of interest, then the measurement can continue. If the measured of a portion of the polynucleotide is indicative of an undesired characteristic, e.g. a genomic region that is not of interest, the polynucleotide can be ejected from the nanopore to allow a further polynucleotide to be characterised. The methods reduce pore blocking in both cases.

In some embodiments, typically where measurements characteristic of the polynucleotide are indicative of one or more desired characteristics of the polynucleotide, the method may comprise repeating the steps of
translocating a portion of the polynucleotide through the nanopore in a first direction with respect to the nanopore, wherein the first direction is from the first opening to the second opening;
contacting the translocated portion of the polynucleotide with one or more conditions which decrease formation of cation-mediated secondary structure; and
translocating the polynucleotide in a second direction with respect to the nanopore, wherein the second direction is from the second opening to the first opening. This allows for a polynucleotide to be characterised repeatedly, which has advantages in improving the characterisation of the polynucleotide, for example improving the base-calling accuracy. In such a manner, the target polynucleotide may oscillate with respect to the detector (i.e. it may be “flossed” with respect to the first and second openings of the detector). This “flossing” allows the target polynucleotide to be repeatedly characterised. In some embodiments this allows the accuracy of the characterisation information to be increased. This is described in more detail in WO 2021/255476, the entire contents of which are hereby incorporated by reference.

The undesired characteristics of a polynucleotide may include the inability of the polynucleotide to fully translocate though the nanopore in the first direction with respect to the nanopore. This characteristic is the detection of a blocked nanopore, which is undesirable as a blocked nanopore prevents the nanopore from characterising further polynucleotides.

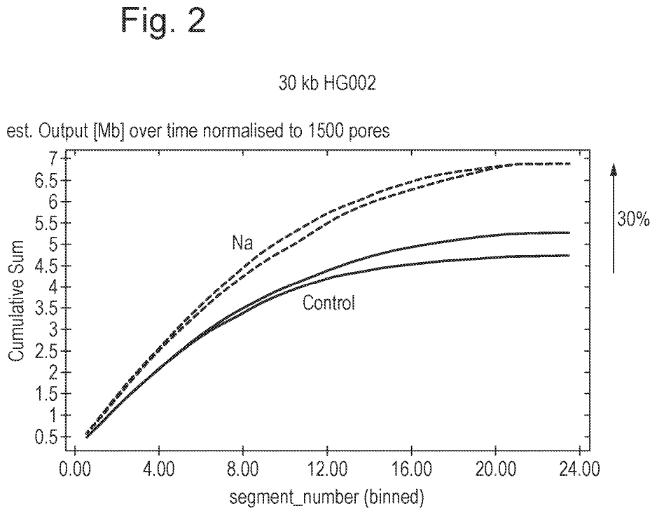

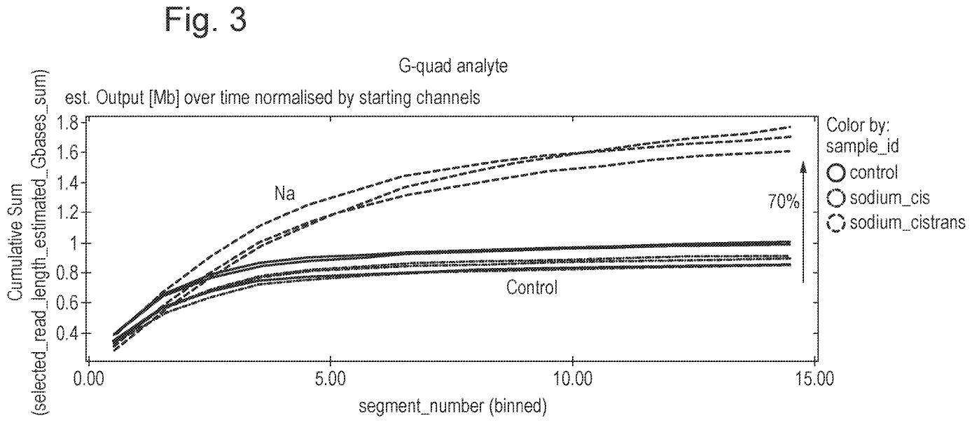

Accordingly, the method of characterising a polynucleotide enables a nanopore to be used to characterise more polynucleotides than would otherwise be possible (due to blockages). In some embodiments, the method comprises characterising a plurality of polynucleotides using a plurality of nanopores. In some embodiments, the plurality of nanopores are present in a nanopore array. The method of characterising a polynucleotide extends the lifetime of the array by reducing nanopore blocking and therefore increasing the availability of nanopores in an array for further characterisation, as shown in FIGS. 2 and 3.

In another aspect, the disclosure relates to methods of operating a nanopore array. Each nanopore in the array has a first opening and a second opening. The method comprises contacting the array with a plurality of polynucleotides under conditions such that a portion of each polynucleotide translocates through the array in a first direction with respect to the nanopores in the array, wherein the first direction is from the first opening to the second opening.

The portion of the polynucleotide that translocates through the nanopore is contacted with one or more conditions which decrease formation of cation-mediated secondary structure, as discussed above with respect to the method of characterising a polynucleotide. The method comprises determining whether each polynucleotide that has been translocated has a desired characteristic, and ejecting polynucleotides which do not have the desired characteristic by translocating the polynucleotide in a second direction with respect to the nanopores in the array, wherein the second direction is from the second opening to the first opening. In some embodiments, the desired characteristic may be the ability of the polynucleotide to fully translocate though the nanopore in the first direction with respect to the nanopore.

By ejecting polynucleotides which do not have the desired characteristic, the efficiency of the nanopore array is improved as time is not spent on measuring characteristics of an undesired polynucleotide. Furthermore, the improved ability to eject polynucleotides that do not have desired characteristics, as a result of the conditions which decrease formation of cation-mediated secondary structure, improves the lifetime of the nanopore array, as discussed above.

In some embodiments of the above methods, the method is performed at a temperature lower than the melting temperature of the cation-mediated secondary structure. In some embodiments, as described in example 1, the stability of cation-mediated secondary structure can be determined using a fluorophore-quencher approach.

As described in example 1, the inventors have determined that under typical running conditions of a nanopore involving a potassium salt, the melting temperature of the exemplary cation-mediated secondary structures may be elevated. The inventors have further determined that conditions which decrease formation of cation-mediated secondary structure can depress the melting temperature of the cation-mediated secondary structure. For example, in example 1 the melting temp of an exemplary G quadruplex was determined to be approximately 65° C., whereas under the conditions identified herein as decreasing formation of cation-mediated secondary structure, the melting temperature of the cation-mediated secondary structure was approximately 50° C. Similarly, in the experimental conditions used in example 2, the melting temperature of an exemplary G quadruplex in the presence of potassium ions was reduced by approximately 10° C. or 20° C. under conditions comprising Na⁺ or R^{b+} cations, respectively.

Surprisingly, the inventors have found that benefits of decreased pore blocking, improved array lifetime and improved data characterisation do not require that the melting temperature of the cation-mediated secondary structure be decreased below the operational temperature of the methods. In other words, without being bound by theory, the inventors consider that the methods disclosed herein provide beneficial effects as disclosed herein even when such methods are operated at temperatures at which cation-mediated secondary structure may still form.

This is particularly advantageous, because raising the operating temperature of a nanopore system to destabilise the formation of the cation-mediated secondary structure may be associated with significant disadvantages, such as altering the stability of other components of the system, e.g. the nanopore itself, as well as the kinetics of polynucleotide translocation, increasing equipment complexity and operational running costs. By simply altering the conditions to decrease formation of cation-mediated secondary structure, these disadvantageous effects are avoided whilst retaining the benefits of pore unblocking.

In some embodiments, the methods may be carried out at from 0° C. to 100° C., such as from 10° C. to 90° C., from 15° C. to 80° C., from 17° C. to 60° C., from 18° C. to 55° C., from 19° C. to 50° C., or from 20° C. to 45° C. In some embodiments the cation mediated secondary structure has a melting temperature of more than 50° C., such as more than 55° C., e.g. more than 60° C.; and the methods are carried out below the melting temperature of the cation mediated secondary structure, e.g. below 60° C., such as below 55° C., e.g. below 50° C.

The methods are typically carried out at a temperature that supports enzyme function, such as a temperature of from about 20° C. to about 50° C. such as from about 30° C. to about 45° C. e.g. from about 34° C. to about 40° C., e.g. about 31, 32, 33, 34, 35, 36, 37, 38, or 39° C.

In some embodiments, the melting temperature of the cation-mediated secondary structure when contacted with the one or more conditions is reduced. In some embodiments, the melting temperature is reduced by at least 5° C., such as at least 10° C., at least 15° C., or at least 20° C. In some embodiments, the reduction in melting temperature may be calculated in comparison to the melting temperature of the cation-mediated secondary structure when contacted with a reference condition. A skilled person can readily determine suitable reference conditions depending on the condition being altered to decrease cation-mediated secondary structure formation. In some embodiments, the reduction in melting temperature is calculated using a thrombin-binding aptamer comprising a G-quadruplex, as known in the art and as discussed in examples 1 and 2.

In some embodiments, the total read output of the nanopore is increased. In some embodiments, the total read output of the nanopore is increased by at least 10%, such as at least 15%, at least 20%, at least 25%, at least 30%, at least 35%, at least 37%, at least 40%, at least 45%, at least 46%, at least 50%, or at least 55%. In some embodiments, the increase in total read output may be calculated in comparison to the total read output of a nanopore comprising a reference condition. A skilled person can readily determine suitable reference conditions depending on the condition being altered to increase total read output of the nanopore. In some embodiments, total read output is calculated over 24, 48 or 72 hours.

In some embodiments, the loss of nanopores in an array is reduced. In this context, the term “loss” is intended to refer to a nanopore that is not capable of taking one or more measurements characteristic of a polynucleotide during the translocation of the polynucleotide through the nanopore. For example, in some embodiments the loss of a nanopore is caused by blocking of the nanopore by the cation-mediated secondary structure of the polynucleotide. In some embodiments, the loss of nanopores in an array is reduced by at least 1%, such as at least 2%, at least 3%, at least 4%, at least 5%, at least 10%, at least 15%, or at least 20%. In some embodiments, the loss of nanopores in an array may be calculated in comparison to the loss of nanopores in an array comprising a reference condition. A skilled person can readily determine suitable reference conditions depending on the condition being altered to reduce the loss of nanopores in an array. In some embodiments, the loss of nanopores in an array is calculated over 24, 48 or 72 hours, as discussed in example 4.

Typically, the reference condition comprises the presence of a potassium salt. The reference conditions may promote formation of potassium cation-mediated secondary structure, such as the formation of potassium cation-mediated G-tetrad and/or G-quadruplex. In some embodiments, the reference condition is the presence of 150 mM potassium ferricyanide, 150 mM potassium ferrocyanide, 25 mM potassium phosphate, pH8 at 34° C.

When the detector is or comprises a nanopore, the first and second openings may be referred to as the cis and trans openings of the nanopore. Often the first opening is the cis opening and the second opening is the trans opening, but in some embodiments the first opening is the trans opening and the second opening is the cis opening, respectively. The notation “cis” and “trans” openings in nanopores is routine in the art. For example, the cis opening of a nanopore typically faces the cis chamber of a nanopore device such as an apparatus as described herein having cis and trans chambers, and the trans opening typically faces the trans chamber.

In some embodiments, the nanopore is present in a structure such as a membrane having a cis side and a trans side. The cis side of the structure or membrane is typically the side of the membrane that is first contacted with the polynucleotide, i.e. the side of a membrane to which a sample comprising the polynucleotide is added. The trans side of the membrane is typically the other side of the membrane from which the polynucleotide is first contacted with the membrane. Typically, the first opening of the nanopore is at the cis side of the membrane and the second opening of the nanopore is at the trans side.

Accordingly, in some embodiments, the first direction is from the cis side of the membrane to the trans side of the nanopore (or structure or membrane comprising the nanopore) and the second direction is from the trans side of the nor (or the structure or membrane comprising the nanopore) to the cis side of the membrane. In some embodiments, the method comprises contacting the translocated portion of the polynucleotide with one or more conditions which decrease formation of cation-mediated secondary structure on the trans side of the membrane.

In some embodiments, the first opening of the nanopore is at the trans side of the nanopore (or structure or membrane comprising the nanopore) and the second opening of the nanopore is at the cis side of the nanopore (or structure or membrane comprising the nanopore). Accordingly, in some embodiments, the first direction is from the trans side of the membrane to the cis side of the membrane and the second direction is from the cis side of the membrane to the trans side of the membrane. In some embodiments, the method comprises contacting the translocated portion of the polynucleotide with one or more conditions which decrease formation of cation-mediated secondary structure on the cis side of the membrane.

In some embodiments the trans side of the membrane comprises the one or more conditions which decrease formation of cation-mediated secondary structure. In some embodiments the conditions on the cis side of the membrane may or may not decrease formation of cation-mediated secondary structure. In some embodiments, the one or more conditions which decrease formation of cation-mediated secondary structure are on both the cis and trans sides of the membrane. In some embodiments, the conditions on the cis side of the membrane comprises one or more conditions that decrease formation of cation-mediated secondary structure. In some embodiments, the conditions on the cis side of the membrane do not comprise conditions that decrease formation of cation-mediated secondary structure.

### Cation-Mediated Secondary Structure

The methods comprise contacting a portion of a polynucleotide that has been translocated through the pore with one or more conditions that decrease formation of cation-mediated secondary structure.

In some embodiments the secondary structure comprises a hydrogen-bonded plurality of polynucleotides. In some embodiments the hydrogen bonded polynucleotides form a planar substructure. In some embodiments a plurality of planar hydrogen-bonded substructures are stabilised by charge interactions. In some embodiments a plurality of such substructures may be stabilised by ionic binding to a metal cation. Such structures may thus be characterised as being cation-mediated.

In some embodiments the secondary structure comprises a plurality of guanosine nucleotides. In some embodiments the secondary structure is formed from a polynucleotide sequence rich in guanosine nucleotides, such as sequence at least 15 nucleotides in length or more and comprising 50% or more guanine bases.

In some embodiments, the cation-mediated secondary structure comprises a tetrad and/or a quadruplex. In some embodiments a tetrad is formed by four nucleotide residues, typically all of the same time, in a planar arrangement. In some embodiments a quadruplex is compared of at least two tetrads stacked upon one another. In some embodiments, the cation-mediated secondary structure comprises a G-tetrad and/or a G-quadruplex. Typically, a G-tetrad is formed by four guanosine nucleotides. Typically, a G-quadruplex is formed by at least two stacked G-tetrads, i.e. by at least 8 guanosine nucleotides. In some embodiments, the G-quadruplex comprises at least two G-tetrads, such as at least three G-tetrads or at least four G-tetrads. G-tetrads and G-quadruplexes are polynucleotide sequences that are rich in guanine and are capable of forming a four-stranded structure. Four guanine bases can associate through Hoogsteen hydrogen bonding to form a square planar structure called a guanine tetrad, and two or more guanine tetrads can stack on top of each other to form a G-quadruplex. The quadruplex structure is further stabilized by the presence of a cation, especially potassium, which sits in a central channel between each pair of tetrads. Forming G-quadruplexes is well known in the art (Marathias and Bolton, Nucleic Acids Research, 2000; 28 (9): 1969-1977; Kankia and Marky, J. Am. Chem. Soc. 2001, 123, 10799-10804; and Marusic et al., Nucleic Acids Research, 2012, 1-11). In some embodiments, the G-quadruplex may be a parallel G-quadruplex, an anti-parallel G-quadruplex or a hybrid G-quadruplex.

In some embodiments, the one or more quadruplexes comprise the sequence Ga followed by Nb followed by Gc followed by Nd followed by Ge followed by Nf followed by Gg, wherein G is a nucleotide comprising guanine, wherein a, c, e and g are independently selected from 1, 2, 3, 4 and 5, wherein N is any nucleotide and wherein b, d and f are from 2 to 50. The values of a, c, e and g may be identical. In some embodiments G is guanosine monophosphate (GMP), cyclic guanosine monophosphate (cGMP), deoxyguanosine monophosphate (dGMP), dideoxyguanosine monophosphate, N2-methyl-GMP, N2-methyl-cGMP, N2-methyl-dGMP, N2-methyl-dideoxyguanosine monophosphate, N2-methyl-06-methyl-GMP, N2-methyl-06-methyl-cGMP, N2-methyl-06-methyl-dGMP, N2-methyl-06-methyl-dideoxyguanosine monophosphate, 2′-O-methyl-GMP, 2′-O-methyl-cGMP, 2′-O-methyl-dGMP, 2′-O-methyl-dideoxyguanosine monophosphate, 6-thio-GMP, 6-thio-cGMP, 6-thio-dGMP, 6-thio-dideoxyguanosine monophosphate, 7-methyl-GMP, 7-methyl-cGMP, 7-methyl-dGMP, 7-methyl-dideoxyguanosine monophosphate, 7-deaza-GMP, 7-deaza-cGMP, 7-deaza-dGMP, 7-deaza-dideoxyguanosine monophosphate, 8-oxo-GMP, 8-oxo-cGMP, 8-oxo-dGMP or 8-oxo-dideoxyguanosine monophosphate. In some embodiments, a G-quadruplex comprises the sequence set out by the formula GₘXₙGₘXₒGₘXₚGₘ, wherein m is the number of G residues of each vertex of the Q quadruplex, and Xn, Xo and Xp can be any combination of residues, including G, forming the loops between the vertices. m is therefore equal to the number of G-tetrads in the G-quadruplex.

In some embodiments, a G-quadruplex comprises at least 60% sequence identity to the sequence GGTTGGTGTGGTTGG (SEQ ID NO: 9), such as at least 70%, at least 80%, at least 90% or 100% sequence identity to the sequence GGTTGGTGTGGTTGG (SEQ ID NO: 9).

The methods comprise decreasing formation of the cation-mediated secondary structure. The conditions may decrease formation of the cation-mediated secondary structure by any amount, such by at least 10%, at least 20%, at least 30%, at least 40%, at least 50%, at least 60%, at least 70%, at least 80% or at least 90%. In some embodiments the methods comprise decreasing stability of the cation-mediated secondary structure. The conditions may decrease the stability of the cation-mediated secondary structure by any amount, such by at least 10%, at least 20%, at least 30%, at least 40%, at least 50%, at least 60%, at least 70%, at least 80% or at least 90%.

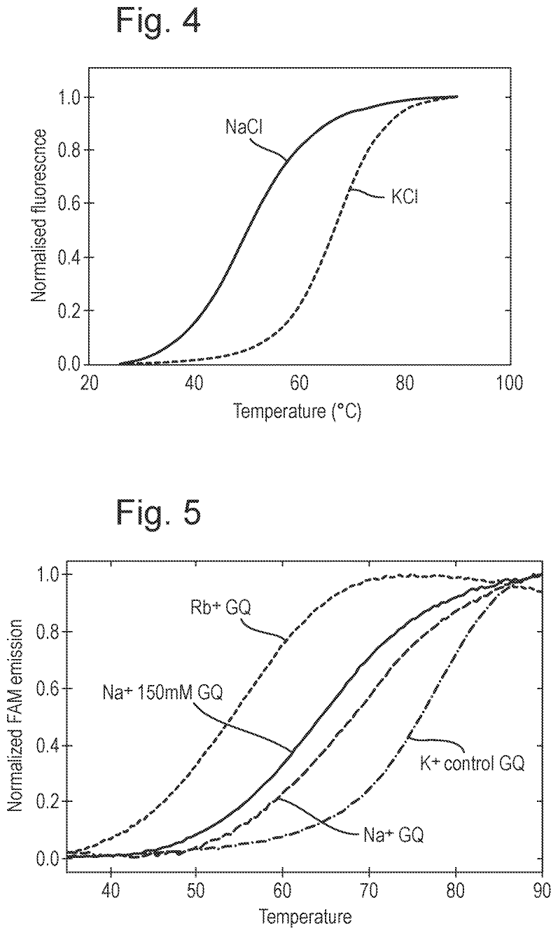

The decrease of cation-mediated secondary structure formation or stability is typically compared to a reference condition. A skilled person can readily determine suitable reference conditions depending on the condition being altered to decrease cation-mediated secondary structure formation. Typically, the reference condition comprises the presence of a potassium salt. The reference conditions may promote formation of potassium cation-mediated secondary structure, such as the formation of potassium cation-mediated G-tetrad and/or G-quadruplex. In some embodiments, the reference condition is the presence of the polynucleotide and 150 mM potassium ferricyanide, 150 mM potassium ferrocyanide, 25 mM potassium phosphate, pH8 at 34° C. A decreased in the formation of the cation-mediated structure may be determined by any means known to the skilled person, for example by identifying the melting temperature of the cation-mediated structure in the one or more conditions and in the reference condition. This is exemplified in FIGS. 4 and 5.

Contacting a Polynucleotide with One or More Conditions

The methods comprise contacting a portion of a polynucleotide that has been translocated through the pore with one or more conditions that decrease formation of cation-mediated secondary structure.

Any suitable conditions can be included. In some embodiments, the methods comprise contacting a portion of a polynucleotide that has been translocated through the pore with two or more conditions that decrease formation of cation-mediated secondary structure, such as three or more, or four or more conditions that decrease formation of cation-mediated secondary structure. Where the first direction is from the cis side of the membrane to the trans side of the membrane, the one or more conditions are typically on the trans side of the membrane. In this embodiment, the cis side of the membrane may also comprise one or more conditions that decrease formation of cation mediated secondary structure. In embodiments where the first direction is from the trans side of the membrane to the cis side of the membrane, the one or more conditions are typically on the cis side of the membrane. In this embodiment, the trans side of the membrane may also comprise one or more conditions that decrease formation of cation mediated secondary structure.

In some embodiments, the conditions include, for example, salt-type, salt concentration, temperature, presence or absence of one or more oligonucleotides, and/or pH.

In some embodiments, the conditions comprise an increased temperature, e.g. when compared to a reference condition. In some embodiments, the method is operated at an increased running temperature, which may decrease the formation and/or stability of cation-mediated secondary structures. For example, a suitable reference temperature may be about 34° C., and in some embodiments, the increased temperature is about 35° C. or higher, such as about 35° C. to about 70° C., e.g. about 35° C. to about 60° C., such as about 40° C. to about 55° C. or about 40° C. to about 50° C.

In some embodiments, the conditions comprise an altered pH, e.g. when compared to reference conditions. For example, a suitable reference pH is about pH 8. In some embodiments, the operational pH is about pH 7 or less, such as from about pH 2 to about pH 7 or about pH 3 to about pH 6. In some embodiments, the operational pH is about pH 9 or more, such as from about pH 9 to about pH 11.

In some embodiments, the conditions comprise one or more control polynucleotides. In some embodiments, the one or more control polynucleotides hybridise with a portion of the sequence that is capable of forming cation-mediated secondary structure. In some embodiments, the one or more control polynucleotides hybridise with a portion of the sequence that is capable of forming a G-tetrad and/or a G-quadruplex. In some embodiments, the one or more control polynucleotides are the reverse complement of the portion of the sequence that is capable of forming cation-mediated secondary structure. In some embodiments, the portion of the sequence to which the one or more control polynucleotide hybridise may be at least 20% of the sequence that is capable of forming cation-mediated secondary structure, such as at least 30%, at least 40%, at least 50%, at least 60%, at least 70%, at least 80%, at least 90% or 100% of the sequence that is capable of forming cation-mediated secondary structure. In some embodiments, the one or more control polynucleotides are at least about 4 nucleotides in length, such as at least about 5, at least about 10, at least about 15, at least about 20, or at least about 30 nucleotides in length. In some embodiments, the one or more control polynucleotides are from about 4-30 nucleotides in length, such as from about 5-25, e.g. from about 10-20 or about 12-16 nucleotides in length.

In some embodiments, the conditions comprise a salt, such as a salt comprising an ammonium cation (NH₄⁺) and/or a metal salt. In some embodiments, the salt disrupts the formation of the cation-mediated secondary structure. In some embodiments, the salt disrupts the stability of the cation-mediated secondary structure. In some embodiments, the salt may comprise one or more cations as described herein.

In some embodiments, the salt comprises an ammonium cation (NH₄⁺). Any suitable anion may be used in the salt. Anions include fluoride, chloride, bromide, iodide, acetate, carbonate, citrate, phosphate, sulphite, sulphate, nitrite, ferricyanide and ferrocyanide. In some embodiments, the salt comprises one or more anions selected from the group consisting of chloride, ferricyanide and ferrocyanide. In some embodiments, the one or more anions comprise chloride. In some embodiments, the one or more anions comprise ferricyanide and ferrocyanide. In some embodiments, the salt comprises ammonium ferricyanide and/or ammonium ferrocyanide, typically a mixture of ammonium ferricyanide and ammonium ferrocyanide. In some embodiments, the salt comprises ammonium chloride. The salt may comprise a mixture of anions described above, such as a mixture of ferricyanide, ferrocyanide and chloride anions. In some embodiments, the salt comprises (i) ammonium chloride and (ii) ammonium ferricyanide and/or ammonium ferrocyanide, typically a mixture of ammonium ferricyanide and ammonium ferrocyanide. In some embodiments, the conditions comprise a salt comprising an ammonium cation and a metal salt, as described herein. In some embodiments, the metal salt comprises one or more cations selected from the group consisting of Rb⁺, Na⁺, Cs⁺, Li⁺, Mg²⁺ and Ca²⁺, as described herein, such as (i) as a chloride salt and/or (ii) as a ferricyanide salt and/or a ferrocyanide salt, typically as a mixture of a ferricyanide salt and ferrocyanide salt

In some embodiments, the concentration of the salt is from about 0.01 to about 1 M, typically from 0.02 to 0.5 M or from 0.05 to 0.3 M. In some embodiments, the concentration of the salt is about 0.025 M, about 0.05 M, about 0.075 M, about 0.1 M, about 0.15 M, about 0.2 M, about 0.25 M or about 0.3 M. Where the salt is a mixture of two or more salts, the total concentration of the salts in the mixture may be from 0.01 to 1 M, typically from 0.02 to 0.5 M or from 0.05 to 0.3 M. In some embodiments, the total concentration of the salt is about 0.025 M, about 0.05 M, about 0.075 M, about 0.1 M, about 0.15 M, about 0.2 M, about 0.25 M or about 0.3 M. In some embodiments, the concentration of the salts in the mixture are identical. In some embodiments, the concentration of the salts in the mixture are not identical. In some embodiments, the concentration of the salts may comprise a ratio of 1:1.5, 1:2, 1:2.5, 1:3, 1:3.5, 1:4 or 1:5. In some embodiments, the salt comprises an ammonium cation and the concentration of the salt is from 0.01 to 1 M, typically from 0.02 to 0.5 M or from 0.05 to 0.3 M, such as about 0.025 M, about 0.05 M, about 0.075 M, about 0.1 M, about 0.15 M, about 0.2 M, about 0.25 M or about 0.3 M. In some embodiments, the salt comprises a mixture of a ferricyanide salt and ferrocyanide salt, and the total concentration of the salts in the mixture is from 0.01 to 1 M, typically from 0.02 to 0.5 M or from 0.05 to 0.3 M, such as about 0.025 M, about 0.05 M, about 0.075 M, about 0.1 M, about 0.15 M, about 0.2 M, about 0.25 M or about 0.3 M.

Typically, the conditions comprise a metal salt. In some embodiments the metal salt disrupts the formation of the cation-mediated secondary structure. In some embodiments the metal salt disrupts the stability of the cation-mediated secondary structure.

In some embodiments, the metal salt comprises one or more monovalent or divalent metal salts. In some embodiments, the metal salt comprises one or more monovalent metal salts.

In some embodiments, the metal salt does not comprise a potassium salt. In some embodiments, the metal salt does not comprise a potassium salt or a magnesium salt. In some embodiments, the metal salt comprises one or more cations having a dehydrated ionic diameter that is larger than the dehydrated ionic diameter of potassium, such as at least 5%, at least 10%, at least 20%, at least 50% or more larger than the dehydrated ionic diameter of potassium. In some embodiments, the metal salt comprises one or more cations having a dehydrated ionic diameter that is smaller than the dehydrated ionic diameter of potassium, such as at least 5%, at least 10%, at least 20%, at least 50% or more smaller than the dehydrated ionic diameter of potassium. In some embodiments, the metal salt comprises one or more cations having a dehydrated ionic diameter that is larger than, or smaller than, the dehydrated ionic diameter of potassium, such as at least 5%, at least 10%, at least 20%, at least 50% or more larger or smaller than the dehydrated ionic diameter of potassium. In some embodiments, the metal salt comprises one or more cations having a hydrated ionic diameter that is larger than the hydrated ionic diameter of potassium, such as at least 5%, at least 10%, at least 20%, at least 50% or more larger. In some embodiments, the metal salt comprises one or more cations having a hydrated ionic diameter that is smaller than the hydrated ionic diameter of potassium, such as at least 5%, at least 10%, at least 20%, at least 50% or more smaller. In some embodiments, the metal salt comprises one or more cations having a hydrated ionic diameter that is larger than, or smaller than, the hydrated ionic diameter of potassium, such as at least 5%, at least 10%, at least 20%, at least 50% or more larger or smaller than the hydrated ionic diameter of potassium. The dehydrated and hydrated sizes of cations are known to the skilled person, for example, as provided in Kielland, J. “Individual activity coefficients of ions in aqueous solutions.” J. Am. Chem. Soc. 59:1675-1678, 1937. Without wishing to be bound by theory, it is believed that a potassium cation may be the optimal size for forming and stabilising cation-mediated secondary structures, such as G-tetrads and G-quadruplexes; such that cations that are larger or smaller than potassium may reduce the formation of, and/or decrease the stability of, the cation-mediated secondary structures.

In some embodiments, the metal salt comprises one or more cations selected from the group consisting of Rb⁺, Na⁺, Cs⁺, Li⁺, Mg²⁺ and Ca²⁺. In some embodiments, the metal salt comprises a cation selected from the group consisting of Na⁺, Cs⁺, Li⁺, Mg²⁺ and Ca²⁺. In some embodiments, the metal salt comprises one or more cations selected from the group consisting of Rb⁺, Na⁺, Cs⁺, Lit and Ca²⁺. In some embodiments, the metal salt comprises one or more cations selected from the group consisting of Rb⁺, Na⁺, Cs⁺ and Lit. Salts comprising each of these cations have been shown to decrease the stability of cation-mediated secondary structure, in particular, G-tetrads and G-quadruplexes. In some embodiments, the metal salt comprises a Rb⁺ cation, i.e. is a rubidium salt. In some embodiments, the metal salt comprises a Na⁺ cation, i.e. is a sodium salt. Any suitable anion may be used in the metal salt. Anions include fluoride, chloride, bromide, iodide, acetate, carbonate, citrate, phosphate, sulphite, sulphate, nitrite, nitrate, ferricyanide and ferrocyanide. In some embodiments, the one or more anions are selected from the group consisting of chloride, ferricyanide and ferrocyanide. In some embodiments, the anion may comprise chloride. In some embodiments, the anion may comprise ferricyanide and ferrocyanide. In some embodiments, the metal salt comprises one or more cations selected from the group consisting of Rb⁺, Na⁺, Cs⁺, Li⁺, Mg²⁺ and Ca²⁺ as a ferricyanide salt and/or a ferrocyanide salt, typically as a mixture of a ferricyanide salt and ferrocyanide salt. In some embodiments, the metal salt comprise a cation selected from the group consisting of Na⁺, Cs⁺, Li⁺, Mg²⁺ and Ca²⁺ as a ferricyanide salt and/or a ferrocyanide salt, typically as a mixture of a ferricyanide salt and ferrocyanide salt. In some embodiments, the metal salt comprises one or more cations selected from the group consisting of Rb⁺, Na⁺, Cs⁺, Li⁺ and Ca²⁺ as a ferricyanide salt and/or a ferrocyanide salt, typically as a mixture of a ferricyanide salt and ferrocyanide salt. In some embodiments, the metal salt comprises one or more cations selected from the group consisting of Rb⁺, Na⁺, Cs⁺ and Lit as a ferricyanide salt and/or a ferrocyanide salt, typically as a mixture of a ferricyanide salt and ferrocyanide salt. In some embodiments, the metal salt comprises rubidium ferricyanide and/or rubidium ferrocyanide, typically a mixture of rubidium ferricyanide and rubidium ferrocyanide. In some embodiments, the metal salt is sodium ferricyanide and/or sodium ferrocyanide, typically a mixture of sodium ferricyanide and sodium ferrocyanide. In some embodiments, the metal salt comprises one or more cations selected from the group consisting of Rb⁺, Na⁺, Cs⁺, Li⁺, Mg²⁺ and Ca²⁺ as a chloride salt. In some embodiments, the metal salt comprise one or more cation selected from the group consisting of Na⁺, Cs⁺, Li⁺, Mg²⁺ and Ca²⁺ as a chloride salt. In some embodiments, the metal salt comprises one or more cations selected from the group consisting of Rb⁺, Na⁺, Cs⁺, Lit and Ca²⁺ as a chloride salt. In some embodiments, the metal salt comprises one or more cations selected from the group consisting of Rb⁺, Na⁺, Cs⁺ and Lit as a chloride salt. In some embodiments, the metal salt comprises rubidium chloride. In some embodiments, the metal salt comprises sodium chloride. The metal salt may comprise a mixture of anions described above, such as a mixture of ferricyanide, ferrocyanide and chloride anions. In some embodiments, the metal salt comprises one or more cations selected from the group consisting of Rb⁺, Na⁺, Cs⁺, Lit, Mg²⁺ and Ca²⁺, and one or more anions comprising (i) chloride and (ii) ferricyanide and/or ferrocyanide, typically a mixture of ferricyanide and ferrocyanide. In some embodiments, the metal salt comprises one or more cations selected from the group consisting of Na⁺, Cs⁺, Li⁺, Mg²⁺ and Ca²⁺, and one or more anions comprising (i) chloride and (ii) ferricyanide and/or ferrocyanide, typically a mixture of ferricyanide and ferrocyanide. In some embodiments, the metal salt comprises one or more cations selected from the group consisting of Rb⁺, Na⁺, Cs⁺, Lit and Ca²⁺, and one or more anions comprising (i) chloride and (ii) ferricyanide and/or ferrocyanide, typically a mixture of ferricyanide and ferrocyanide. In some embodiments, the metal salt comprises one or more cations selected from the group consisting of Rb⁺, Na⁺, Cs⁺ and Lit, and one or more anions comprising (i) chloride and (ii) ferricyanide and/or ferrocyanide, typically a mixture of ferricyanide and ferrocyanide. In some embodiments, the metal salt comprises (i) rubidium chloride and (ii) rubidium ferricyanide and/or rubidium ferrocyanide, typically a mixture of rubidium ferricyanide and rubidium ferrocyanide. In some embodiments, the metal salt comprises (i) sodium chloride and (ii) sodium ferricyanide and/or sodium ferrocyanide, typically a mixture of sodium ferricyanide and sodium ferrocyanide. The beneficial effects identified by the inventors when the metal salt comprises a cation selected from the group consisting of Rb⁺, Na⁺, Cs⁺, Li⁺, Mg²⁺ and Ca²⁺ are a feature of the present disclosure. In more detail, and without being bound by theory, the signal: noise ratio of measurements taken in a nanopore system as a polynucleotide moves with respect to a nanopore may correlate with base-calling accuracy. Conditions which decrease formation of cation-mediated secondary structure are conventionally associated with a decreased signal: noise ratio compared to corresponding conditions comprising potassium salts. However, the inventors have surprisingly found that base-calling accuracy of nanopore measurements may be retained even under conditions which decrease formation of cation-mediated secondary structure.

In some embodiments, the concentration of the metal salt is from about 0.01 to about 1 M, typically from 0.02 to 0.5 M or from 0.05 to 0.3 M. In some embodiments, the concentration of the metal salt is about 0.025 M, about 0.05 M, about 0.075 M, about 0.1 M, about 0.15 M, about 0.2 M, about 0.25 M or about 0.3 M. Where the metal salt is a mixture of two or more salts, the total concentration of the metal salts in the mixture is from 0.01 to 1 M, typically from 0.02 to 0.5 M or from 0.05 to 0.3 M. In some embodiments, the total concentration of the metal salt is about 0.025 M, about 0.05 M, about 0.075 M, about 0.1 M, about 0.15 M, about 0.2 M, about 0.25 M or about 0.3 M. In some embodiments, the concentration of the metal salts in the mixture are identical. In some embodiments, the concentration of the metal salts in the mixture are not identical. In some embodiments, the concentration of the metal salts may comprise a ratio of 1:1.5, 1:2, 1:2.5, 1:3, 1:3.5, 1:4 or 1:5. In some embodiments, the metal salt comprises a Na⁺ cation and the concentration of the metal salt is from 0.01 to 1 M, typically from 0.02 to 0.5 M or from 0.05 to 0.3 M, such as about 0.025 M, about 0.05 M, about 0.075 M, about 0.1 M, about 0.15 M, about 0.2 M, about 0.25 M or about 0.3 M. In some embodiments, the metal salt comprises a Rb⁺ cation and the concentration of the metal salt is from 0.01 to 1 M, typically from 0.02 to 0.5 M or from 0.05 to 0.3 M, such as about 0.025 M, about 0.05 M, about 0.075 M, about 0.1 M, about 0.15 M, about 0.2 M, about 0.25 M or about 0.3 M. In some embodiments, the metal salt comprises a mixture of a ferricyanide salt and ferrocyanide salt, and the total concentration of the metal salts in the mixture is from 0.01 to 1 M, typically from 0.02 to 0.5 M or from 0.05 to 0.3 M, such as about 0.025 M, about 0.05 M, about 0.075 M, about 0.1 M, about 0.15 M, about 0.2 M, about 0.25 M or about 0.3 M. In some embodiments, the metal salt comprises about 0.01 to about 1 M rubidium ferricyanide, typically from 0.02 to 0.5 M, from 0.05 to 0.3 M, from 0.1 to 0.2 M, or about 0.15 M rubidium ferricyanide. In some embodiments, the metal salt comprises about 0.01 to about 1 M rubidium ferrocyanide, typically from 0.02 to 0.5 M, from 0.05 to 0.3 M, from 0.1 to 0.2 M, or about 0.15 M rubidium ferrocyanide. In some embodiments, the metal salt comprises about 0.005 to about 0.1 M rubidium phosphate, typically from 0.01 to 0.05 M, from 0.02 to 0.03 M, or about 0.025 M rubidium phosphate. In some embodiments, the metal salt comprises about 0.01 to about 1 M rubidium ferricyanide, 0.01 to about 1 M rubidium ferrocyanide and about 0.005 to about 0.1 M rubidium phosphate, typically: from 0.02 to 0.5 M rubidium ferricyanide, from 0.02 to 0.5 M rubidium ferrocyanide and 0.005 to 0.01 M rubidium phosphate; from 0.05 to 0.3 M rubidium ferricyanide, from 0.05 to 0.3 M rubidium ferrocyanide and 0.01 to 0.05 M rubidium phosphate; from 0.1 to 0.2 M rubidium ferricyanide, from 0.1 to 0.2 M rubidium ferrocyanide and 0.02 to 0.03 M rubidium phosphate; or about 0.15 M rubidium ferricyanide, about 0.15 M rubidium ferrocyanide and about 0.025 M rubidium phosphate. In some embodiments, the metal salt comprises about 0.01 to about 1 M sodium ferricyanide, typically from 0.02 to 0.5 M, from 0.05 to 0.3 M, from 0.1 to 0.2 M, or about 0.15 M or about 0.21 M sodium ferricyanide. In some embodiments, the metal salt comprises about 0.01 to about 1 M sodium ferrocyanide, typically from 0.02 to 0.5 M, from 0.05 to 0.3 M, from 0.1 to 0.2 M, or about 0.15 M or about 0.21 M sodium ferrocyanide. In some embodiments, the metal salt comprises about 0.005 to about 0.1 M sodium phosphate, typically from 0.01 to 0.05 M, from 0.02 to 0.03 M, or about 0.025 M sodium phosphate. In some embodiments, the metal salt comprises about 0.01 to about 1 M sodium ferricyanide, 0.01 to about 1 M sodium ferrocyanide and about 0.005 to about 0.1 M sodium phosphate, typically: from 0.02 to 0.5 M sodium ferricyanide, from 0.02 to 0.5 M sodium ferrocyanide and 0.005 to 0.01 M sodium phosphate; from 0.05 to 0.3 M sodium ferricyanide, from 0.05 to 0.3 M sodium ferrocyanide and 0.01 to 0.05 M sodium phosphate; from 0.1 to 0.2 M sodium ferricyanide, from 0.1 to 0.2 M sodium ferrocyanide and 0.02 to 0.03 M sodium phosphate; about 0.15 M sodium ferricyanide, about 0.15 M sodium ferrocyanide and about 0.025 M sodium phosphate; or about 0.21 M sodium ferricyanide, about 0.21 M sodium ferrocyanide and about 0.025 M sodium phosphate.

The methods are typically carried out in the presence of charge carriers, as discussed herein. In some embodiments, the conditions comprise a further metal salt as a charge carrier. Typically, the additional metal salt is not a potassium salt. In some embodiments, the further metal salt comprises one or more monovalent or divalent metal salts. In some embodiments, the further metal salt comprises one or more monovalent metal salts. In some embodiments, the further metal salt comprises one or more cations selected from the group consisting of Rb⁺, Na⁺, Cs⁺, Li⁺, Mg²⁺ and Ca²⁺, typically Rb⁺ or Na⁺. In some embodiments, the further metal salt comprises a cation selected from the group consisting of Na⁺, Cs⁺, Li⁺, Mg²⁺ and Ca²⁺, typically Na⁺. In some embodiments, the further metal salt comprises one or more cations selected from the group consisting of Rb⁺, Na⁺, Cs⁺, Lit and Ca²⁺. In some embodiments, the further metal salt comprises one or more cations selected from the group consisting of Rb⁺, Na⁺, Cs⁺ and Lit. In some embodiments, the further metal salt is a chloride salt. In some embodiments, the further metal salt comprises one or more chloride salts comprising a cation selected from the group consisting of Rb⁺, Na⁺, Cs⁺, Li⁺, Mg²⁺ and Ca²⁺, typically Rb⁺ or Na⁺. In some embodiments, the further metal salt is a chloride salt comprising a cation selected from the group consisting of Na⁺, Cs⁺, Li⁺, Mg²⁺ and Ca²⁺, typically Na⁺. In some embodiments, the further metal salt comprises one or more chloride salts comprising a cation selected from the group consisting of Rb⁺, Na⁺, Cs⁺, Lit, and Ca²⁺. In some embodiments, the further metal salt comprises one or more chloride salts comprising a cation selected from the group consisting of Rb⁺, Na⁺, Cs⁺ and Lit. In some embodiments, the further metal salt comprises RbCl. In some embodiments the further metal salt is NaCl. In some embodiments, the concentration of the further metal salt is at saturation. In some embodiments, the concentration of the further metal salt is 3 M or lower, typically from 0.1 to 2.5 M, from 0.3 to 1.9 M, from 0.5 to 1.8 M, from 0.7 to 1.7 M, from 0.9 to 1.6 M or from 1 M to 1.4 M. The concentration of the further metal salt is preferably from 150 mM to 1 M. The method is preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High concentrations of the further metal salt provide a high signal to noise ratio and allow for currents indicative of the presence of a nucleotide to be identified against the background of normal current fluctuations.

As discussed above, the decrease of cation-mediated secondary structure formation is typically compared to a reference condition. In some embodiments, the reference condition comprises a potassium salt. In some embodiments, the method is conducted under conditions comprising 500 mM or less of potassium salt, such as 100 mM potassium salt or less, 10 mM potassium salt or less, 1 mM potassium salt or less, 1 μM potassium salt or less, or 100 nM potassium salt or less. In some embodiments, 50% or less, such as 10% or less, e.g. 1% or less of the salt in the conditions is a potassium salt, such as 0.1% or less, 0.01% or less, or 0.001% or less. In some embodiments, the conditions are substantially free of a potassium salt. A condition is substantially free of potassium salt when potassium cation-mediated secondary structure is absent or undetectable by the use of standard biochemical techniques.

The one or more conditions which decrease formation of cation-mediated secondary structure are contacted with the portion of the polynucleotide that has translocated through the nanopore. In some embodiments, the one or more conditions on each side of the membrane are symmetric, i.e. the one or more conditions are the same on the cis and the trans sides of the membrane. In some embodiments, the one or more conditions on each side of the membrane comprise a salt as described herein, such as a salt comprising an ammonium cation (NH₄⁺) and/or a metal salt. In some embodiments, the one or more conditions on each side of the membrane comprise a metal salt as described herein. In some embodiments, the one or more conditions on each side of the membrane may comprise one or more cations selected from the group consisting of Rb⁺, Na⁺, Cs⁺, Li⁺, Mg²⁺ and Ca²⁺. In some embodiments, the one or more conditions on each side of the membrane may comprise one or more cations selected from the group consisting of Na⁺, Cs⁺, Li⁺, Mg²⁺ and Ca²⁺. In some embodiments, the one or more conditions on each side of the membrane may comprise one or more cations selected from the group consisting of Rb⁺, Na⁺, Cs⁺, Li⁺ and Ca²⁺. In some embodiments, the one or more conditions on each side of the membrane may comprise one or more cations selected from the group consisting of Rb⁺, Na⁺, Cs⁺ and Lit. In some embodiments, the one or more conditions on each side of the membrane may comprise a Rb⁺ cation. In some embodiments, the one or more conditions on each side of the membrane may comprise a Na⁺ cation.

In some embodiments, the one or more conditions of each side of the membrane are asymmetric. In embodiments where the first direction is from the cis side of the membrane to the trans side of the membrane, the one or more conditions are on the trans side of the membrane. In this embodiment, the conditions of the cis side of the membrane may comprise a potassium salt. In embodiments where the first direction is from the trans side of the membrane to the cis side of the membrane, the one or more conditions are on the cis side of the membrane. In this embodiment, the conditions of the trans side of the membrane may comprise a potassium salt. In some embodiments, the trans side of the membrane may comprise one or more conditions as described herein, and the cis side of the membrane may comprise a different set of one or more conditions as described herein. In embodiments where the first direction is from the cis side of the membrane to the trans side of the membrane, the one or more conditions on the trans side of the membrane may comprise a salt as described herein, such as a salt comprising an ammonium cation (NH₄⁺) and/or a metal salt comprising one or more cations selected from the group consisting of Rb⁺, Na⁺, Cs⁺, Li⁺, Mg²⁺ and Ca²⁺. In this embodiment, the conditions on the cis side of the membrane may or may not comprise one or more conditions decrease formation of cation-mediated secondary structure. In embodiments where the first direction is from the trans side of the membrane to the cis side of the membrane, the one or more conditions on the cis side of the membrane may comprise a salt as described herein, such as a salt comprising an ammonium cation (NH₄⁺) and/or a metal salt comprising one or more cations selected from the group consisting of Rb⁺, Na⁺, Cs⁺, Li⁺, Mg²⁺ and Ca²⁺. In this embodiment, the conditions on the trans side of the membrane may or may not comprise one or more conditions decrease formation of cation-mediated secondary structure.

### Translocating a Polynucleotide

The methods comprise translocating a portion of a polynucleotide through a nanopore in a first direction with respect to the nanopore, wherein the first direction is from the first opening to the second opening, and translocating the polynucleotide in a second direction with respect to the nanopore, wherein the second direction is from the second opening to the first opening.

In some embodiments the nanopore is in a membrane having a cis side and a trans side, the first opening of the nanopore is at the cis side of the membrane and the second opening of the nanopore is at the trans side, and a portion of the polynucleotide translocates though the nanopore in a first direction from the cis side to the trans side of the membrane. In some embodiments the nanopore is in a membrane having a cis side and a trans side, the first opening of the nanopore is at the trans side of the membrane and the second opening of the nanopore is at the cis side, and a portion of the polynucleotide translocates though the nanopore in a first direction from the trans side to the cis side of the membrane.

A portion of the polynucleotide is typically sufficient in length to form a cation-mediated secondary structure. In some embodiments, the portion of the polynucleotide is 15 nucleotides in length or more, such as 25 nucleotides in length or more, 50 nucleotides in length or more, 100 nucleotides in length or more, 500 nucleotides in length or more, 1000 nucleotides in length or more, 5000 nucleotides in length or more, 10000 nucleotides in length or more, 50000 nucleotides in length or more, or 100000 nucleotides in length or more. In some embodiments, the portion of the polynucleotide is at least 1% of the total length of the polynucleotide, such as at least 5% of the total length of the polynucleotide, at least 10% of the total length of the polynucleotide, at least 20% of the total length of the polynucleotide, at least 30% of the total length of the polynucleotide, at least 40% of the total length of the polynucleotide, at least 50% of the total length of the polynucleotide, at least 60% of the total length of the polynucleotide, at least 70% of the total length of the polynucleotide, at least 80% of the total length of the polynucleotide, of the total length of the polynucleotide, at least 90% of the total length of the polynucleotide, or at least 95% of the total length of the polynucleotide. In some embodiments, the portion is the full length of the polynucleotide, i.e. the entire polynucleotide is translocated through the nanopore. As used herein, the term “translocating” relates to the movement of a polynucleotide in the first direction or the second direction with respect to the nanopore, i.e. does not necessarily require that the full polynucleotide passes through the nanopore and is subsequently released.

In some embodiments, the portion of the polynucleotide comprises a sequence that is capable of forming a cation-mediated secondary structure as described herein (such as a G quadruplex) under reference conditions. Typically, the reference condition comprises a potassium salt, as discussed herein.

In some embodiments, the methods comprise controlling the movement of the polynucleotide, or of each polynucleotide, with respect to the nanopore in the first direction using a polynucleotide-handling protein. In some embodiments, the methods comprises controlling the movement of the polynucleotide, or of each polynucleotide, with respect to the nanopore in the second direction using a polynucleotide-handling protein. In some embodiments, the methods comprise controlling the movement of the polynucleotide, or of each polynucleotide, with respect to the nanopore in the first direction using a polynucleotide-handling protein and controlling the movement of the polynucleotide, or of each polynucleotide, with respect to the nanopore in the second direction using a polynucleotide-handling protein.

### Characteristics of a Polynucleotide

In some embodiments, the methods comprise taking one or more measurements characteristic of the polynucleotide during the translocation of the polynucleotide through the nanopore. This step thereby characterises the polynucleotide.

When used to characterize the polynucleotide, the presence, absence or one or more characteristics of the target polynucleotide are determined. In some embodiments, the methods are for determining the presence, absence or one or more characteristics of a polynucleotide. In some embodiments, the methods concern determining the presence, absence or one or more characteristics of two or more polynucleotides. The methods may comprise determining the presence, absence or one or more characteristics of any number of polynucleotides, such as 2, 5, 10, 15, 20, 30, 40, 50, 100 or more polynucleotides. Any number of characteristics of the one or more target polynucleotides may be determined, such as 1, 2, 3, 4, 5, 10 or more characteristics. Characteristics amenable to being detected in the methods provide herein include (i) the length of the polynucleotide, (ii) the identity of the polynucleotide, (iii) the sequence of the polynucleotide, (iv) the secondary structure of the polynucleotide and (v) whether or not the polynucleotide is modified. In some embodiments the methods provided herein are methods of sequencing a target polynucleotide. In some embodiments, a polynucleotide sequence may be determined in real-time by aligning real-time signal or basecalling to known references. Exemplary methods of determining a polynucleotide sequence are described in WO 2016/059427, incorporated by reference herein.

In some embodiments, the methods comprise determining if a measured characteristic of the polynucleotide is indicative of one or more desired or undesired characteristics of the polynucleotides.

In some embodiments, the methods comprise determining the presence or absence of one or more desired characteristic. In some embodiments, if the measurements characteristic of the polynucleotide are indicative of a desired characteristic, the method comprises fully translocating the polynucleotide in the first direction. In some embodiments, if the measurements characteristic of the polynucleotide are indicative of a desired characteristic, the method comprises repeating the translocation of the polynucleotide in the first and the second directions multiple times. This allows re-reading approaches as described in WO 2021/255476, incorporated herein by reference, to be applied to the disclosed methods, as described above. The desired characteristic may be the ability of the polynucleotide to translocate through the pore. The desired characteristic may be the sequence of a polynucleotide of interest, for example a particular genomic region.

In some embodiments, the methods comprise determining the presence or absence of one or more undesired characteristic. In some embodiments, if the measurements characteristic of the polynucleotide are indicative of an undesired characteristic, the method comprises ejecting the polynucleotide from the first opening of the nanopore. In some embodiments, ejecting the polynucleotide comprises translocating the portion of the polynucleotide through the nanopore in the second direction. In some embodiments, the one or more undesired characteristic includes the inability of the polynucleotide to fully translocate through the nanopore in the first direction with respect to the nanopore, i.e. the polynucleotide blocks the pore. In some embodiments, a method of characterising a polynucleotide comprises translocating a portion of the polynucleotide through the nanopore in the first direction with respect to the nanopore, determining the presence or absence of the inability of the polynucleotide to fully translocate through the nanopore in the first direction, wherein if the polynucleotide is unable to fully translocate through the nanopore in the first direction with respect to the nanopore, the method further comprises ejecting the polynucleotide from the first opening of the nanopore translocating the portion of the polynucleotide through the nanopore in the second direction. In some embodiments, the one or more undesired characteristic is the sequence of a polynucleotide that is not of interest. The method may comprise ejecting the polynucleotide with the undesired characteristic from the first opening of the nanopore. This allows adaptive sampling approaches as described in WO 2016/059427, incorporated herein by reference, to be applied to the disclosed methods.

In some embodiments, the methods comprise:
translocating a portion of a polynucleotide through the nanopore in a first direction with respect to the nanopore;
determining whether the translocated polynucleotide has a desired characteristic or an undesired characteristic;
ejecting the polynucleotide if it does not have the desired characteristic or if it has an undesired characteristic;
taking one or more further measurements characteristic of the polynucleotide if it has a desired characteristic or if it does not have an undesired characteristic.

In some embodiments, therefore, the nanopore is in a membrane having a cis side and a trans side, the first opening of the nanopore is at the cis side of the membrane and the second opening of the nanopore is at the trans side, a portion of the polynucleotide translocates though the nanopore in a first direction from the cis side to the trans side of the membrane; the translocated portion of the polynucleotide is contacted with one or more conditions which decrease formation of cation-mediated secondary structure; and the polynucleotide is translocated in a second direction from the trans side to the cis side of the membrane; and the method further comprises taking one or more measurements characteristic of the polynucleotide during the translocation of the polynucleotide through the nanopore. In some embodiments, if the measurements characteristic of the polynucleotide are indicative of one or more desired characteristics of the polynucleotide, the method comprises translocating the entire polynucleotide through the nanopore in the first direction from the cis side to the trans side of the membrane, and taking one or more measurements during said translocation; whereas if the measurements characteristic of the polynucleotide are indicative of one or more undesired characteristics of the polynucleotide, the method comprises ejecting the polynucleotide from the first opening of the nanopore by translocating the portion of the polynucleotide in the second direction from the trans side to the cis side of the membrane.

Similarly, in other embodiments, the nanopore is in a membrane having a cis side and a trans side, the first opening of the nanopore is at the trans side of the membrane and the second opening of the nanopore is at the cis side, a portion of the polynucleotide translocates though the nanopore in a first direction from the trans side to the cis side of the membrane; the translocated portion of the polynucleotide is contacted with one or more conditions which decrease formation of cation-mediated secondary structure; and the polynucleotide is translocated in a second direction from the cis side to the trans side of the membrane; and the method further comprises taking one or more measurements characteristic of the polynucleotide during the translocation of the polynucleotide through the nanopore. In some embodiments, if the measurements characteristic of the polynucleotide are indicative of one or more desired characteristics of the polynucleotide, the method comprises translocating the entire polynucleotide through the nanopore in the first direction from the trans side to the cis side of the membrane, and taking one or more measurements during said translocation; whereas if the measurements characteristic of the polynucleotide are indicative of one or more undesired characteristics of the polynucleotide, the method comprises ejecting the polynucleotide from the first opening of the nanopore by translocating the portion of the polynucleotide in the second direction from the cis side to the trans side of the membrane.

### Ejecting a Polynucleotide

In some embodiments, the methods comprise ejecting a polynucleotide, particularly is the polynucleotide comprises measured characteristics that are indicative of one or more undesired characteristic or the lack of a desired characteristic. In the methods disclosed herein, the polynucleotide is ejected from the first opening of the nanopore. Accordingly, to eject the polynucleotide, the polynucleotide is translocated in the second direction with respect to the nanopore. The full polynucleotide is translocated through the nanopore in the second direction such that a further polynucleotide may be contacted with the pore and translocated in the first direction and/or in the second direction.

In some embodiments the polynucleotide is ejected from the nanopore by applying a force across the nanopore which causes the polynucleotide to move in the second direction, i.e. in the direction from the second opening of the nanopore to the first opening of the nanopore. In some embodiments the force is a voltage force. In some embodiments the ejection force is opposite in direction to a voltage force applied across the membrane during the translocation of the polynucleotide in the first direction, i.e. from the first opening of the nanopore to the second opening of the nanopore. In some embodiments the translocation in the first direction is carried out under a positive voltage and the ejection voltage is a negative voltage. In some embodiments the voltage is applied at the trans side of the nanopore relative to the cis side of the nanopore. In some embodiments the translocation in the first direction is carried out under a negative voltage and the ejection voltage is a positive voltage. In some embodiments the voltage is applied at the cis side of the nanopore relative to the trans side of the nanopore.

### Polynucleotide

In some embodiments, the methods disclosed herein involve characterising a polynucleotide.

A polynucleotide, such as a nucleic acid, is a macromolecule comprising two or more nucleotides. A polynucleotide can be single-stranded or double-stranded. A double-stranded polynucleotide is made of two single stranded polynucleotides hybridised together. The polynucleotide can be a single-stranded polynucleotide or a double-stranded polynucleotide as described in more detail herein. The portion of the polynucleotide that translocates through the pore in the first direction is typically a single-stranded portion of the polynucleotide.

A polynucleotide may comprise any combination of any nucleotides. The nucleotides can be naturally occurring or artificial.

A nucleotide typically contains a nucleobase, a sugar and at least one phosphate group. The nucleobase and sugar form a nucleoside.

The nucleobase is typically heterocyclic. Nucleobases include, but are not limited to, purines and pyrimidines and more specifically adenine (A), guanine (G), thymine (T), uracil (U) and cytosine (C).

The sugar is typically a pentose sugar. Nucleotide sugars include, but are not limited to, ribose and deoxyribose. The sugar is preferably a deoxyribose. The polynucleotide preferably comprises the following nucleosides: deoxyadenosine (dA), deoxyuridine (dU) and/or thymidine (dT), deoxyguanosine (dG) and deoxycytidine (dC).

The nucleotide is typically a ribonucleotide or deoxyribonucleotide. The nucleotide typically contains a monophosphate, diphosphate or triphosphate. The nucleotide may comprise more than three phosphates, such as 4 or 5 phosphates. Phosphates may be attached on the 5′ or 3′ side of a nucleotide. Nucleotides include, but are not limited to, adenosine monophosphate (AMP), guanosine monophosphate (GMP), thymidine monophosphate (TMP), uridine monophosphate (UMP), 5-methylcytidine monophosphate, 5-hydroxymethylcytidine monophosphate, cytidine monophosphate (CMP), cyclic adenosine monophosphate (CAMP), cyclic guanosine monophosphate (cGMP), deoxyadenosine monophosphate (dAMP), deoxyguanosine monophosphate (dGMP), deoxythymidine monophosphate (dTMP), deoxyuridine monophosphate (dUMP), deoxycytidine monophosphate (dCMP) and deoxymethylcytidine monophosphate. The nucleotides are preferably selected from AMP, TMP, GMP, CMP, UMP, dAMP, dTMP, dGMP, dCMP and dUMP.

A nucleotide may be abasic (i.e. lack a nucleobase). A nucleotide may also lack a nucleobase and a sugar (i.e. is a C3 spacer).

The polynucleotide can be any length. In some embodiments, the polynucleotide is at least 100, at least 150, at least 200, at least 250, at least 300, at least 400 or at least 500 nucleotides or nucleotide pairs in length. In some embodiments, the polynucleotide can be 1000 or more nucleotides or nucleotide pairs in length, 5000 or more nucleotides or nucleotide pairs in length or 100000 or more nucleotides or nucleotide pairs in length or 500,000 or more nucleotides or nucleotide pairs in length, or 1,000,000 or more nucleotides or nucleotide pairs in length, 10,000,000 or more nucleotides or nucleotide pairs in length, or 100,000,000 or more nucleotides or nucleotide pairs in length, or 200,000,000 or more nucleotides or nucleotide pairs in length, or the entire length of a chromosome. Longer polynucleotides, such as those at least 500 nucleotides or nucleotide pairs in length have particular use in the methods disclosed herein, as the chance of encountering a sequence that may form a cation-mediated secondary structure increases and thus the advantages of the methods are most easily recognisable. In embodiments wherein the method disclosed herein involves a plurality of polynucleotides, the plurality of polynucleotides may have an average (e.g. a median) length of at least 5,000 or more nucleotides or nucleotide pairs in length, or at least 10,000 or more nucleotides or nucleotide pairs in length, or at least 15,000 or more nucleotides or nucleotide pairs in length, or at least 20,000 or more nucleotides or nucleotide pairs in length, or at least 30,000 or more nucleotides or nucleotide pairs in length, or at least 100,000 or more nucleotides or nucleotide pairs in length, or at least 500,000 or more nucleotides or nucleotide pairs in length, or at least 1,000,000 or more nucleotides or nucleotide pairs in length, or at least 10,000,000 or more nucleotides or nucleotide pairs in length, or at least 100,000,000 or more nucleotides or nucleotide pairs in length, or at least 200,000,000 or more nucleotides or nucleotide pairs in length. The methods disclosed herein are of particular use for characterising polynucleotides of a longer average (e.g. median) length, such as those at least 5,000 nucleotides or nucleotide pairs in length, particularly wherein the plurality of polynucleotides are derived from a genomic sample, and the frequency of occurrence of cation-mediated secondary structure is unknown.

The polynucleotide may be a fragment of a longer polynucleotide. In this embodiment, the longer polynucleotide is typically fragmented into multiple, such as two or more, shorter polynucleotides.

In some embodiments, the polynucleotide comprises a sequence that is capable of forming cation-mediated secondary structure, such as a G-tetrad and/or a G-quadruplex. In some embodiments, the polynucleotide comprises a sequence that is rich in guanosine nucleotides which is capable of forming cation-mediated secondary structure, such as a G-tetrad and/or a G-quadruplex. Typically, a sequence rich in guanosine nucleotides is 15 nucleotides in length or more and comprises 50% or more guanine bases.

The nucleotides in the polynucleotide may be attached to each other in any manner. The nucleotides are typically attached by their sugar and phosphate groups as in nucleic acids. The nucleotides may be connected via their nucleobases as in pyrimidine dimers.

The polynucleotide can be a nucleic acid, such as deoxyribonucleic acid (DNA) or ribonucleic acid (RNA). The polynucleotide can comprise one strand of RNA hybridized to one strand of DNA. The polynucleotide may be any synthetic nucleic acid known in the art, such as peptide nucleic acid (PNA), glycerol nucleic acid (GNA), threose nucleic acid (TNA), locked nucleic acid (LNA), bridged nucleic acid (BNA) or other synthetic polymers with nucleotide side chains. The PNA backbone is composed of repeating N-(2-aminoethyl)-glycine units linked by peptide bonds. The GNA backbone is composed of repeating glycol units linked by phosphodiester bonds. The TNA backbone is composed of repeating threose sugars linked together by phosphodiester bonds. LNA is formed from ribonucleotides as discussed above having an extra bridge connecting the 2′ oxygen and 4′ carbon in the ribose moiety.

The polynucleotide is typically DNA, RNA or a DNA or RNA hybrid, most preferably DNA. A DNA/RNA hybrid may comprise DNA and RNA on the same strand. Preferably, the DNA/RNA hybrid comprises one DNA strand hybridized to an RNA strand.

The backbone of the polynucleotide can be altered to reduce the possibility of strand scission. For example, DNA is known to be more stable than RNA under many conditions. The backbone of the polynucleotide strand can be modified to avoid damage caused by e.g. harsh chemicals such as free radicals.

DNA or RNA that contains unnatural or modified bases can be produced by amplifying natural DNA or RNA polynucleotides in the presence of modified NTPs using an appropriate polymerase.

The nucleotides in the polynucleotide may be modified. The nucleotides may be oxidized or methylated. One or more nucleotides in the polynucleotide may be damaged. For instance, the polynucleotide may comprise a pyrimidine dimer. Such dimers are typically associated with damage by ultraviolet light and are the primary cause of skin melanomas. One or more nucleotides in the polynucleotide may be modified with a label or a tag.

In some embodiments, the polynucleotide is a double-stranded polynucleotide. In some embodiments, prior to contacting the polynucleotide with a nanopore, the polynucleotide is comprised in or consists of a first strand of a double-stranded polynucleotide comprising said first strand and a second strand.

In some embodiments, the polynucleotide is a double-stranded polynucleotide and the first strand is hybridised to the second strand.

In some embodiments, the two strands of a double-stranded molecule are attached together. For example, in embodiments wherein the target polynucleotide comprises a first strand and a second strand, the first strand may be attached to the second strand. The two strands may be covalently linked, for example at the ends of the molecules by joining the 5′ end of one strand to the 3′ end of the other with a hairpin structure.

The polynucleotide may comprise the products of a PCR reaction, genomic DNA, the products of an endonuclease digestion and/or a DNA library.

The polynucleotide may be naturally occurring. The polynucleotide may be secreted from cells. Alternatively, the polynucleotide may be present inside cells such that the polynucleotide must be extracted from the cells before the method can be carried out.

The polynucleotide may be sourced from common organisms such as viruses, bacteria, archaea, plants or animals. Such organisms may be selected or altered to adjust the sequence of the target polynucleotide, for example by adjusting the base composition, removing unwanted sequence elements, and the like. The selection and alteration of organisms in order to arrive at desired polynucleotide characteristics is routine for one of ordinary skill in the art.

The source organism for the polynucleotide may be chosen based on desired characteristics of the sequence. Desired characteristics include the ratio of single-stranded vs double-stranded polynucleotides produced by the organism; the complexity of the sequences of polynucleotides produced by the organism, the composition of the polynucleotides produced by the organism (such as the GC composition), or the length of contiguous polynucleotide strands produced by the organism. For example, when a contiguous polynucleotide strand of around 50 kb is required, lambda phage DNA can be used. If longer contiguous strands are required, other organisms can be used to produce the polynucleotide; for example E. coli produces around 4.5 Mb of contiguous dsDNA.

The polynucleotide is typically obtained from a human or animal, e.g. from urine, lymph, saliva, mucus, seminal fluid or amniotic fluid, or from whole blood, plasma or serum. The polynucleotide may be obtained from a plant e.g. a cereal, legume, fruit or vegetable. The polynucleotide may comprise genomic DNA. The genomic DNA may be fragmented. The DNA may be fragmented by any suitable method. For example, methods of fragmenting DNA are known in the art. Such methods may use a transposase, such as a MuA transposase. Often the genomic DNA is not fragmented.

In some embodiments the polynucleotide is synthetic or semi-synthetic. For example, DNA or RNA may be purely synthetic, synthesised by conventional DNA synthesis methods such as phosphoramidite based chemistries. Synthetic polynucleotides subunits may be joined together by known means, such as ligation or chemical linkage, to produce longer strands. In some embodiments, internal self-forming structures (e.g. hairpins, quadruplexes) can be designed into the substrate e.g. by ligating appropriate sequences. Synthetic polynucleotides can be copied and scaled up for production by means known in the art, including PCR, incorporation into bacterial factories, and the like.

In some embodiments, the polynucleotide may have a simplified nucleotide composition. In some embodiments the polynucleotide has a repeating pattern of the same subunit. For example, a repeating unit may be (AmGn)q, wherein m, n and q are positive integers. For example, m is often from 1 to 20, such as from 1 to 10 e.g. from 1 to 5, e.g. 1, 2, 3, 4 or 5. n is often from 1 to 20, such as from 1 to 10 e.g. from 1 to 5, e.g. 1, 2, 3, 4 or 5. m and n may be the same or different. q is often from 1 to about 100,000. A typical repeating unit may be for example (AAAAAAGGGGGG)q. Repeating polynucleotides can be made by many means known in the art, for example by concatenating together synthetic subunits with sticky ends that enable ligation. In some embodiments, the polynucleotide may therefore be a concatenated polynucleotide. Methods of concatenating polynucleotides are described in PCT/GB2017/051493.

Any number of polynucleotides can be used in the disclosed methods. For instance, the method may comprise using 2, 3, 4, 5, 6, 7, 8, 9, 10, 20, 30, 50, 100 or more polynucleotides. The disclosed methods reduce the chances of pore blocking by a polynucleotide by enabling the polynucleotide blocking the nanopore to be ejected, thus allowing additional polynucleotides to be used. If two or more polynucleotides are used, they may be different polynucleotides or two instances of the same polynucleotide. The polynucleotide can be naturally occurring or artificial.

It is within the scope of the methods provided herein that the polynucleotide is labelled with a molecular label. A molecular label may be a modification to the polynucleotide which promotes the detection of the polynucleotide in the methods provided herein. For example, the label may interfere with a flux of ions through the nanopore. In such a manner, the label may improve the sensitivity of the methods.

### Adapter

In some embodiments of the methods provided herein, a polynucleotide may have a polynucleotide adapter attached thereto. An adapter typically comprises a polynucleotide strand capable of being attached to the end of the polynucleotide. Accordingly, in some embodiments the methods comprise attaching an adapter (e.g. an adapter as described herein) to a polynucleotide. In one embodiment, the adapter is synthetic or artificial. Typically, the adapter comprises a polymer as described herein. In some embodiments, the adapter comprises a spacer as described herein. In some embodiments, the adapter comprises a polynucleotide. The polynucleotide adapter may comprise DNA, RNA, modified DNA (such as abasic DNA), RNA, PNA, LNA, BNA and/or PEG. Usually, the adapter comprises single stranded and/or double stranded DNA or RNA. The adapter may comprise the same type of polynucleotide as the polynucleotide strand to which it is attached. The adapter may comprise a different type of polynucleotide to the polynucleotide strand to which it is attached. In some embodiments, the polynucleotide used in the disclosed methods is a single stranded DNA strand and the adapter comprises DNA or RNA, typically single stranded DNA. In some embodiments, the polynucleotide is a double stranded DNA strand and the adapter comprises DNA or RNA, e.g. double or single stranded DNA.

In some embodiments, an adapter may be a bridging moiety. A bridging moiety may be used to connect the two strands of a double-stranded polynucleotide. For example, in some embodiments a bridging moiety is used to connect the template strand of a double stranded polynucleotide to the complement strand of the double stranded polynucleotide.

A bridging moiety typically covalently links the two strands of a double-stranded polynucleotide. The bridging moiety can be anything that is capable of linking the two strands of a double-stranded polynucleotide, provided that the bridging moiety does not interfere with movement of the polynucleotide with respect to the nanopore. Suitable bridging moieties include, but are not limited to a polymeric linker, a chemical linker, a polynucleotide or a polypeptide. Preferably, the bridging moiety comprises DNA, RNA, modified DNA (such as abasic DNA), RNA, PNA, LNA or PEG. The bridging moiety is more preferably DNA or RNA.

In some embodiments a bridging moiety is a hairpin adapter. A hairpin adapter is an adapter comprising a single polynucleotide strand, wherein the ends of the polynucleotide strand are capable of hybridising to each other, or are hybridized to each other, and wherein the middle section of the polynucleotide forms a loop. Suitable hairpin adapters can be designed using methods known in the art. In some embodiments a hairpin loop is typically 4 to 100 nucleotides in length, e.g. from 4 to 50 such as from 4 to 20 e.g. from 4 to 8 nucleotides in length. In some embodiments, the bridging moiety (e.g. hairpin adapter) is attached at one end of a double-stranded polynucleotide. A bridging moiety (e.g. hairpin adapter) is typically not attached at both ends of a double-stranded polynucleotide.

In some embodiments of the methods provided herein, a polynucleotide may have a sequencing adapter attached thereto. A sequencing adapter is typically loaded with a polynucleotide-handling protein, such as a motor protein. The polynucleotide-handling protein may be stalled at a spacer on the sequencing adapter. Examples of suitable sequencing adapters are disclosed in WO 2015/110813 and WO 2020/234612, which are herein incorporated by reference.

In some embodiments an adapter is a linear adapter. A linear adapter may be bound to either or both ends of a single stranded polynucleotide. When the polynucleotide is a double stranded polynucleotide, a linear adapter may be bound to either or both ends of either or both strands of the double stranded polynucleotide.

A linear adapter may comprise a leader sequence as described herein. A linear adapter may comprise a portion for hybridisation with a tag (such as a pore tag) as described herein. A linear adapter may be 10 to 150 nucleotides in length, such as from 20 to 120, e.g. 30 to 100, for example 40 to 80 such as 50 to 70 nucleotides in length. A linear adapter may be single stranded. A linear adapter may be double stranded.

In some embodiments, an adapter may be a Y adapter. A Y adapter is typically a polynucleotide adapter. A Y adapter is typically double stranded and comprises (a) at one end, a region where the two strands are hybridised together and (b), at the other end, a region where the two strands are not complementary. The non-complementary parts of the strands typically form overhangs. The presence of a non-complementary region in the Y adapter gives the adapter its Y shape since the two strands typically do not hybridise to each other unlike the double stranded portion. The two single-stranded portions of the Y adapter may be the same length, or may be different lengths. For example, one single-stranded portion of the Y adapter may be 10 to 150 nucleotides in length, such as from 20 to 120, e.g. 30 to 100, for example 40 to 80 such as 50 to 70 nucleotides in length and the other single stranded portion of the Y adapter may independently by 10 to 150 nucleotides in length, such as from 20 to 120, e.g. 30 to 100, for example 40 to 80 such as 50 to 70 nucleotides in length. The double-stranded “stem” portion of the Y adapter may be e.g. from 10 to 150 nucleotides in length, such as from 20 to 120, e.g. 30 to 100, for example 40 to 80 such as 50 to 70 nucleotides in length. A Y adapter may be attached to either or both ends of the construct described herein.

An adapter may be linked to a polynucleotide by any suitable means known in the art. The adapter may be synthesized separately and chemically attached or enzymatically ligated to the polynucleotide. Alternatively, the adapter may be generated in the processing of the polynucleotide. In some embodiments, the adapter is linked to the polynucleotide at or near one end of the target polynucleotide. In some embodiments, the adapter is linked to the polynucleotide within 50, e.g. within 20 for example within 10 nucleotides of an end of the polynucleotide. In some embodiments the adapter is linked to the polynucleotide at a terminus of the polynucleotide. When an adapter is linked to the polynucleotide the adapter may comprise the same type of nucleotides as the polynucleotide or may comprise different nucleotides to the polynucleotide.

### Spacer

In some embodiments of the methods provided herein, a polynucleotide or an adapter as described herein may comprise a spacer. For example, one or more spacers may be present in a polynucleotide adapter.

In some embodiments, a polynucleotide or polynucleotide adapter may comprise one or more spacers, e.g. from one to about 10 spacers, e.g. from 1 to about 5 spacers, e.g. 1, 2, 3, 4 or 5 spacers. The spacer may comprise any suitable number of spacer units. A spacer typically provides an energy barrier which impedes movement of a polynucleotide binding protein. For example, a spacer may impede movement of a polynucleotide-handling protein by reducing the traction of the protein, e.g. using an abasic spacer. A spacer may physically block movement of the protein, for instance by introducing a bulky chemical group to physically impede the movement of the polynucleotide-handling protein.

In some embodiments, one or more spacers are included in the polynucleotide or in a polynucleotide adapter to provide a distinctive signal when they pass through or across a nanopore. One or more spacers may be used to define or separate one or more regions of a polynucleotide; e.g. to separate an adapter from the target polynucleotide.

In some embodiments, a spacer may comprise a linear molecule, such as a polymer, e.g. a polypeptide or a polyethylene glycol (PEG). Typically, such a spacer has a different structure from the target polynucleotide. For instance, if the target polynucleotide is DNA, the or each spacer typically does not comprise DNA. In particular, if the target polynucleotide is deoxyribonucleic acid (DNA) or ribonucleic acid (RNA), the or each spacer preferably comprises peptide nucleic acid (PNA), glycerol nucleic acid (GNA), threose nucleic acid (TNA), locked nucleic acid (LNA) or a synthetic polymer with nucleotide side chains. In some embodiments, a spacer may comprise one or more nitroindoles, one or more inosines, one or more acridines, one or more 2-aminopurines, one or more 2-6-diaminopurines, one or more 5-bromo-deoxyuridines, one or more inverted thymidines (inverted dTs), one or more inverted dideoxy-thymidines (ddTs), one or more dideoxy-cytidines (ddCs), one or more 5-methylcytidines, one or more 5-hydroxymethylcytidines, one or more 2′-O-Methyl RNA bases, one or more Iso-deoxycytidines (Iso-dCs), one or more Iso-deoxyguanosines (Iso-dGs), one or more C3 (OC₃H₆OPO₃) groups, one or more photo-cleavable (PC) [OC₃H₆—C(O) NHCH₂-C₆H₃NO₂—CH(CH₃)OPO₃] groups, one or more hexandiol groups, one or more spacer 9 (iSp9) [(OCH₂CH₂)₃OPO₃] groups, or one or more spacer 18 (iSp18) [(OCH₂CH₂)₆OPO₃] groups; or one or more thiol connections. A spacer may comprise any combination of these groups. Many of these groups are commercially available from IDT® (Integrated DNA Technologies®). For example, C3, iSp9 and iSp18 spacers are all available from IDT®. A spacer may comprise any number of the above groups as spacer units.

In some embodiments, a spacer may comprise one or more chemical groups, e.g. one or more pendant chemical groups. The one or more chemical groups may be attached to one or more nucleobases in a polynucleotide adapter. The one or more chemical groups may be attached to the backbone of a polynucleotide adapter. Any number of appropriate chemical groups may be present, such as 2, 3, 4, 5, 6, 7, 8, 9, 10, 11, 12 or more. Suitable groups include, but are not limited to, fluorophores, streptavidin and/or biotin, cholesterol, methylene blue, dinitrophenols (DNPs), digoxigenin and/or anti-digoxigenin and dibenzylcyclooctyne groups.

In some embodiments, a spacer may comprise one or more abasic nucleotides (i.e. nucleotides lacking a nucleobase), such as 2, 3, 4, 5, 6, 7, 8, 9, 10, 11, 12 or more abasic nucleotides. The nucleobase can be replaced by —H (idSp) or —OH in the abasic nucleotide. Abasic spacers can be inserted into target polynucleotides by removing the nucleobases from one or more adjacent nucleotides. For instance, polynucleotides may be modified to include 3-methyladenine, 7-methylguanine, 1,N6-ethenoadenine inosine or hypoxanthine and the nucleobases may be removed from these nucleotides using Human Alkyladenine DNA Glycosylase (hAAG). Alternatively, polynucleotides may be modified to include uracil and the nucleobases removed with Uracil-DNA Glycosylase (UDG). In one embodiment, the one or more spacers do not comprise any abasic nucleotides.

Methods of stalling a polynucleotide-handling protein such as a helicase on a polynucleotide adapter using a spacer are described in WO 2014/135838, which is hereby incorporated by reference in its entirety.

Suitable spacers can be designed or selected depending on the nature of the polynucleotide or polynucleotide adapter, the polynucleotide-handling protein and the conditions under which the method is to be carried out.

### Anchor

In some embodiments, a polynucleotide or an adapter attached thereto may comprise a membrane anchor or a transmembrane pore anchor e.g. attached to the adapter. In one embodiment the anchor aids in characterisation of the polynucleotide in accordance with the methods disclosed herein. For example, a membrane anchor or transmembrane pore anchor may promote localisation of the polynucleotide around a nanopore in a membrane.

In one embodiment, a polynucleotide or polynucleotide adapter may comprise a membrane anchor or a transmembrane pore anchor. In one embodiment the anchor assists in the characterisation of a polynucleotide in accordance with the methods disclosed herein. For example, a membrane anchor or transmembrane pore anchor may promote localisation of the polynucleotides with a desired characteristic around a nanopore.

The anchor may be a polypeptide anchor and/or a hydrophobic anchor that can be inserted into the membrane. In one embodiment, the hydrophobic anchor is a lipid, fatty acid, sterol, carbon nanotube, polypeptide, protein or amino acid, for example cholesterol, palmitate or tocopherol. The anchor may comprise thiol, biotin or a surfactant. In one aspect the anchor may be biotin (for binding to streptavidin), amylose (for binding to maltose binding protein or a fusion protein), Ni-NTA (for binding to poly-histidine or poly-histidine tagged proteins) or peptides (such as an antigen).

In one embodiment, the anchor comprises a linker, or 2, 3, 4 or more linkers. Preferred linkers include, but are not limited to, polymers, such as polynucleotides, polyethylene glycols (PEGs), polysaccharides and polypeptides. These linkers may be linear, branched or circular. For instance, the linker may be a circular polynucleotide. The adapter may hybridise to a complementary sequence on a circular polynucleotide linker. The one or more anchors or one or more linkers may comprise a component that can be cut or broken down, such as a restriction site or a photolabile group. The linker may be functionalised with maleimide groups to attach to cysteine residues in proteins. Suitable linkers are described in WO 2010/086602.

In one embodiment, the anchor is cholesterol or a fatty acyl chain. For example, any fatty acyl chain having a length of from 6 to 30 carbon atom, such as hexadecanoic acid, may be used. Examples of suitable anchors and methods of attaching anchors to adapters are disclosed in WO 2012/164270 and WO 2015/150786.

In another embodiment the anchor may consist or comprise a hydrophobic modification to the polynucleotide or polynucleotide adapter. The hydrophobic modification may comprise a modified phosphate group comprised within the polynucleotide or polynucleotide anchor. The hydrophobic modification may for example comprise a phosphorothioate such as a charge-neutralized alkyl-phosphorothioate (PPT) as described in Jones et al, J. Am. Chem. Soc. 2021, 143, 22, 8305, the entire contents of which are hereby incorporated by reference. Suitable alkyl groups include for example C₁-C₁₀ alkyl groups such as C₂-C₆ alkyl groups; e.g. methyl, ethyl, propyl, butyl, pentyl and hexyl groups. Incorporation of the charge-neutralized alkyl-phosphorothioate into a polynucleotide allows for the polynucleotide to anchor to a hydrophobic region such as a lipid bilayer.

### Polynucleotide-Handling Enzyme

In some embodiments of the methods disclosed herein, the translocation of the polynucleotide through the pore in the first direction and/or in the second direction with respect to the nanopore (preferably in the first direction) is controlled using a polynucleotide handling enzyme. In some embodiments the polynucleotide-handling protein controls the movement of the polynucleotide in the same direction as the physical or chemical force (potential). For example, in some embodiments a positive voltage potential is applied to the trans side of the nanopore, and the polynucleotide-handling protein controls the movement of the construct from the cis side of the nanopore to the trans side of the nanopore. In some embodiments a positive voltage potential is applied to the cis side of the nanopore relative to the trans side of the nanopore, and the polynucleotide-handling protein controls the movement of the construct from the trans side of the nanopore to the cis side of the nanopore.

In some embodiments the polynucleotide-handling protein controls the movement of the polynucleotide in the opposite direction to the physical or chemical force (potential). For example, in some embodiments a positive voltage potential is applied to the trans side of the nanopore, and the polynucleotide-handling protein controls the movement of the construct from the trans side of the nanopore to the cis side of the nanopore. In some embodiments a positive voltage potential is applied to the cis side of the nanopore, and the polynucleotide-handling protein controls the movement of the construct from the cis side of the nanopore to the trans side of the nanopore.

In some embodiments the movement of the polynucleotide is driven by the polynucleotide-handling protein in the absence of an applied potential.

In some embodiments, the disclosed methods comprise contacting the polynucleotide with a polynucleotide-handling protein capable of controlling the movement of the polynucleotide through the pore in the first direction and/or in the second direction with respect to the nanopore.

Suitable polynucleotide-handling proteins are also known as motor proteins or polynucleotide-handling enzymes. Suitable polynucleotide-handling proteins are known in the art and some exemplary polynucleotide-handling proteins are described in more detail below. In some embodiments, the polynucleotide-handling protein is, comprises, or is derived from a helicase, translocate or helicase-nuclease complex.

In one embodiment, a motor protein is or is derived from a polynucleotide handling enzyme. A polynucleotide handling enzyme is a polypeptide that is capable of interacting with and modifying at least one property of a polynucleotide. The enzyme may modify the polynucleotide by cleaving it to form individual nucleotides or shorter chains of nucleotides, such as di- or trinucleotides. The enzyme may modify the polynucleotide by orienting it or moving it to a specific position.

In some embodiments, a polynucleotide-handling protein may be modified to prevent it from disengaging from a polynucleotide (other than by passing off the end of the strand) during the methods provided herein. Modification of a polynucleotide-handling protein in order to prevent it from disengaging from a polynucleotide can be achieved using methods known in the art, such as those discussed in WO 2014/013260, which is hereby incorporated by reference in its entirety, and with particular reference to passages describing the modification of polynucleotide-handling proteins (polynucleotide binding proteins) such as helicases in order to prevent them from disengaging with polynucleotide strands.

The polynucleotide-handling protein may be chosen or selected according to the polynucleotide to be used in the methods disclosed herein. Alternatively, the polynucleotide may be chosen or selected according to the polynucleotide-handling protein used in the disclosed methods. For example, typically DNA motor proteins can be used when the polynucleotide is DNA. RNA motor protein can be used when the polynucleotide is RNA. Motor proteins which can process both DNA and RNA can be used when the polynucleotide is a hybrid of DNA and RNA.

In one embodiment, the motor protein is derived from a member of any of the Enzyme Classification (EC) groups 3.1.11, 3.1.13, 3.1.14, 3.1.15, 3.1.16, 3.1.21, 3.1.22, 3.1.25, 3.1.26, 3.1.27, 3.1.30 and 3.1.31.

In some embodiments of the claimed methods, the motor protein is a helicase, a polymerase, an exonuclease, a topoisomerase, or a variant thereof.

In one embodiment, the motor protein is an exonuclease. Suitable enzymes include, but are not limited to, exonuclease I from E. coli (SEQ ID NO: 1), exonuclease III enzyme from E. coli (SEQ ID NO: 2), RecJ from T. thermophilus (SEQ ID NO: 3) and bacteriophage lambda exonuclease (SEQ ID NO: 4), TatD exonuclease and variants thereof. Three subunits comprising the sequence shown in SEQ ID NO: 3 or a variant thereof interact to form a trimer exonuclease.

In one embodiment, the motor protein is a polymerase. The polymerase may be PyroPhage® 3173 DNA Polymerase (which is commercially available from Lucigen® Corporation), SD Polymerase (commercially available from Bioron®), Klenow from NEB or variants thereof. In one embodiment, the enzyme is Phi29 DNA polymerase (SEQ ID NO: 5) or a variant thereof. Modified versions of Phi29 polymerase that may be used in the disclosed methods are disclosed in U.S. Pat. No. 5,576,204.

In embodiments of the methods provided herein which comprise controlling the movement of the polynucleotide by synthesizing a strand complementary to a polynucleotide strand, the polynucleotide-handling protein is typically a polymerase, e.g. a polymerase as described herein.

In one embodiment the polynucleotide-handling protein is a topoisomerase. In one embodiment, the topoisomerase is a member of any of the Moiety Classification (EC) groups 5.99.1.2 and 5.99.1.3. The topoisomerase may be a reverse transcriptase, which are enzymes capable of catalysing the formation of cDNA from a RNA template. They are commercially available from, for instance, New England Biolabs® and Invitrogen®.

In one embodiment the polynucleotide-handling protein is a translocase. Examples include translocases in the FtsK and SpoIII families.

In one embodiment, the polynucleotide-handling protein is a helicase. Any suitable helicase can be used in accordance with the methods provided herein. For example, the or each motor protein used in accordance with the present disclosure may be independently selected from a Hel308 helicase, a RecD helicase, a Tral helicase, a TrwC helicase, an XPD helicase, and a Dda helicase, or a variant thereof. Monomeric helicases may comprise several domains attached together. For instance, Tral helicases and Tral subgroup helicases may contain two RecD helicase domains, a relaxase domain and a C-terminal domain. The domains typically form a monomeric helicase that is capable of functioning without forming oligomers. Particular examples of suitable helicases include Hel308, NS3, Dda, UvrD, Rep, PcrA, Pif1 and TraI. These helicases typically work on single stranded DNA. Examples of helicases that can move along both strands of a double stranded DNA include FtsK and hexameric enzyme complexes, or multisubunit complexes such as RecBCD, and are particularly suited to some embodiments disclosed herein. NS3 helicases are particularly suitable for use in the disclosed methods as they are capable of processing both DNA and RNA and so can be used in embodiments of the disclosed methods in which the target double stranded nucleic acid is a DNA-RNA hybrid.

Hel308 helicases are described in publications such as WO 2013/057495, the entire contents of which are incorporated by reference. RecD helicases are described in publications such as WO 2013/098562, the entire contents of which are incorporated by reference. XPD helicases are described in publications such as WO 2013/098561, the entire contents of which are incorporated by reference. Dda helicases are described in publications such as WO 2015/055981 and WO 2016/055777, the entire contents of each of which are incorporated by reference.

In one embodiment the helicase comprises the sequence shown in SEQ ID NO: 6 (Trwc Cba) or a variant thereof, the sequence shown in SEQ ID NO: 7 (Hel308 Mbu) or a variant thereof or the sequence shown in SEQ ID NO: 8 (Dda) or a variant thereof. Variants may differ from the native sequences in any of the ways discussed herein. An example variant of SEQ ID NO: 8 comprises E94C/A360C. A further example variant of SEQ ID NO: 8 comprises E94C/A360C and then (ΔM1)G1G2 (i.e. deletion of M1 and then addition of G1 and G2).

In some embodiments a motor protein (e.g. a helicase) can control the movement of the polynucleotide in at least two active modes of operation (when the motor protein is provided with all the necessary components to facilitate movement, e.g. fuel and cofactors such as ATP and Mg²⁺ discussed herein) and one inactive mode of operation (when the motor protein is not provided with the necessary components to facilitate movement).

When provided with all the necessary components to facilitate movement (i.e. in the active modes), the motor protein (e.g. helicase) moves along the polynucleotide in a 5′ to 3′ or a 3′ to 5′ direction (depending on the motor protein). The motor protein can be used to either move the polynucleotide away from (e.g. out of) the pore (e.g. against an applied force) or towards (e.g. into) the pore (e.g. with an applied force). For example, when the end of the polynucleotide strand towards which the motor protein moves is captured by a pore, the motor protein works against the direction of the force and pulls the threaded strand out of the pore (e.g. into the cis chamber). However, when the end away from which the motor protein moves is captured in the pore, the motor protein works with the direction of the force and pushes the threaded strand into the pore (e.g. into the trans chamber).

When the motor protein (e.g. helicase) is not provided with the necessary components to facilitate movement (i.e. in the inactive mode) it can bind to the polynucleotide and act as a brake slowing the movement of the strand when it is moved with respect to a nanopore, e.g. by being pulled into the pore by a force. In the inactive mode, it does not matter which end of the strand is captured, it is the applied force which determines the movement with respect to the pore, and the polynucleotide binding protein acts as a brake. When in the inactive mode, the movement control by the polynucleotide binding protein can be described in a number of ways including ratcheting, sliding and braking.

A motor protein typically requires fuel in order to handle the processing of polynucleotides. Fuel is typically free nucleotides or free nucleotide analogues. The free nucleotides may be one or more of, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), cyclic adenosine monophosphate (CAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP). The free nucleotides are usually selected from AMP, TMP, GMP, CMP, UMP, dAMP, dTMP, dGMP or dCMP. The free nucleotides are typically adenosine triphosphate (ATP).

A cofactor for the motor protein is a factor that allows the motor protein to function. The cofactor is preferably a divalent metal cation. The divalent metal cation is preferably Mg²⁺, Mn²⁺, Ca²⁺ or Co²⁺. The cofactor is most preferably Mg²⁺.

### Nanopore

In the disclosed methods, any suitable nanopore can be used. In some embodiments, the nanopore is a protein pore, a solid state pore or a DNA origami pore. In some embodiments, the nanopore is a transmembrane pore, such as a transmembrane protein pore. The pore may be biological or artificial.

A transmembrane pore is a structure that crosses the membrane to some degree. It permits hydrated ions driven by an applied potential to flow across or within the membrane. The transmembrane pore typically crosses the entire membrane so that hydrated ions may flow from one side of the membrane to the other side of the membrane. However, the transmembrane pore does not have to cross the membrane. It may be closed at one end. For instance, the pore may be a well, gap, channel, trench or slit in the membrane along which or into which hydrated ions may flow.

In the methods provided herein, the nanopore typically has a first opening and a second opening. As discussed herein, the first opening is typically the cis opening and the second opening is typically the trans opening. However, in some embodiments the first opening is the trans opening and the second opening is the cis opening. The polynucleotide-handling enzyme used in some embodiments of the methods provided herein is typically provided at the first opening of the nanopore and thus controls the movement of the target polynucleotide in the first direction.

A solid state pore may, in one embodiment, comprise a nanochannel. In some embodiments, the solid state pore is a pore disclosed in WO 2003/003446, WO 2009/020682 or WO 2016/187519, each of which is incorporated by reference in their entirety.

In one embodiment, the pore may be a DNA origami pore (Langecker et al., Science, 2012; 338:932-936). Suitable DNA origami pores are disclosed in WO2013/083983, WO 2018/011603 and WO 2020/025974, each of which is incorporated by reference in their entirety.

In one embodiment, the nanopore is a scaffolded polypeptide nanopore. In some embodiments the pore is a scaffolded polypeptide nanopore as disclosed in WO 2020/025909 or WO 2020/074399, each of which is incorporated by reference in their entirety.

In one embodiment, the nanopore is a transmembrane protein pore. A transmembrane protein pore is a polypeptide or a collection of polypeptides that permits hydrated ions, such as polynucleotide, to flow from one side of a membrane to the other side of the membrane. In the methods provided herein, the transmembrane protein pore is capable of forming a pore that permits hydrated ions driven by an applied potential to flow from one side of the membrane to the other. The transmembrane protein pore preferably permits polynucleotides to flow from one side of the membrane, such as a triblock copolymer membrane, to the other. The transmembrane protein pore allows a polynucleotide to be moved through the pore.

In one embodiment, the nanopore is a transmembrane protein pore which is a monomer or an oligomer. The pore is preferably made up of several repeating subunits, such as at least 6, at least 7, at least 8, at least 9, at least 10, at least 11, at least 12, at least 13, at least 14, at least 15, or at least 16 subunits. The pore is preferably a hexameric, heptameric, octameric or nonameric pore. The pore may be a homo-oligomer or a hetero-oligomer.

In one embodiment, the transmembrane protein pore comprises a barrel or channel through which the ions may flow. The subunits of the pore typically surround a central axis and contribute strands to a transmembrane B-barrel or channel or a transmembrane cx-helix bundle or channel.

Typically, the barrel or channel of the transmembrane protein pore comprises amino acids that facilitate interaction with an analyte, such as a target polynucleotide (as described herein). These amino acids are preferably located near a constriction of the barrel or channel. The transmembrane protein pore typically comprises one or more positively charged amino acids, such as arginine, lysine or histidine, or aromatic amino acids, such as tyrosine or tryptophan. These amino acids typically facilitate the interaction between the pore and nucleotides, polynucleotides or nucleic acids.

In one embodiment, the nanopore is a transmembrane protein pore derived from β-barrel pores or α-helix bundle pores. B-barrel pores comprise a barrel or channel that is formed from B-strands. Suitable B-barrel pores include, but are not limited to, B-toxins, such as α-hemolysin, anthrax toxin and leukocidins, and outer membrane proteins/porins of bacteria, such as Mycobacterium smegmatis porin (Msp), for example MspA, MspB, MspC or MspD, CsgG, outer membrane porin F (OmpF), outer membrane porin G (OmpG), outer membrane phospholipase A and Neisseria autotransporter lipoprotein (NalP) and other pores, such as lysenin. α-helix bundle pores comprise a barrel or channel that is formed from α-helices. Suitable α-helix bundle pores include, but are not limited to, inner membrane proteins and α outer membrane proteins, such as WZA and ClyA toxin.

In one embodiment the nanopore is a transmembrane pore derived from or based on Msp, α-hemolysin (α-HL), lysenin, CsgG, ClyA, Sp1 or haemolytic protein fragaceatoxin C (FraC).

In one embodiment, the nanopore is a transmembrane protein pore derived from CsgG, e.g. from CsgG from E. coli Str. K-12 substr. MC4100. Such a pore is oligomeric and typically comprises 7, 8, 9 or 10 monomers derived from CsgG. The pore may be a homo-oligomeric pore derived from CsgG comprising identical monomers. Alternatively, the pore may be a hetero-oligomeric pore derived from CsgG comprising at least one monomer that differs from the others. Examples of suitable pores derived from CsgG are disclosed in WO 2016/034591, WO 2017/149316, WO 2017/149317, WO 2017/149318 and WO 2019/002893, each of which is hereby incorporated by reference in its entirety.

In one embodiment, the nanopore is a transmembrane pore derived from lysenin. Examples of suitable pores derived from lysenin are disclosed in WO 2013/153359, which is hereby incorporated by reference in its entirety.

In one embodiment, the nanopore is a transmembrane pore derived from or based on α-hemolysin (α-HL). The wild type α-hemolysin pore is formed of 7 identical monomers or sub-units (i.e., it is heptameric). An α-hemolysin pore may be α-hemolysin-NN or a variant thereof. The variant preferably comprises N residues at positions E111 and K147.

In one embodiment, the nanopore is a transmembrane protein pore derived from Msp, e.g. from MspA. Examples of suitable pores derived from MspA are disclosed in WO 2012/107778.

In one embodiment, the nanopore is a transmembrane pore derived from or based on ClyA. Examples of suitable pores derived from ClyA are disclosed in Soskine et al., Nano Letters 2012 12 (9), 4895-4900; WO 2014/153625; and WO 2017/098322, each of which is hereby incorporated by reference.

In one embodiment, the nanopore is a transmembrane pore derived from Phi29. Examples of suitable pores derived from Phi29 are disclosed in Wendell et al., Nature Nanotech 4, 765-772 (2009), WO 2010/062697, WO 2019/157365 and WO 2019/157424, each of which is hereby incorporated by reference.

In some embodiments the nanopore is selected from M-ring protein, perforin-2, PlyAB (pleurotolysin), SpoIIIAG, VirB7, Type II secretion system protein D, GspD, InvG, PilQ, pentraxin, and portal proteins including T4, T7, P23_45, G20c and Phi29 nanopores.

In one embodiment, the nanopore is a transmembrane pore derived from or based on a Rhodococcus species of bacteria, for example Rhodococcus corynebacteroides or Rhodococcus ruber, for example PorARr, PorBRr or PorARc. Examples of such pores are described in Piselli et al., Eur Biophys J 51, 309-323 (2022).

### Membrane

In the disclosed methods, the nanopore is typically a nanopore present in a membrane. Any suitable membrane may be used.

The membrane is preferably an amphiphilic layer. An amphiphilic layer is a layer formed from amphiphilic molecules, such as phospholipids, which have both hydrophilic and lipophilic properties. The amphiphilic molecules may be synthetic or naturally occurring. Non-naturally occurring amphiphiles and amphiphiles which form a monolayer are known in the art and include, for example, block copolymers (Gonzalez-Perez et al., Langmuir, 2009, 25, 10447-10450). Block copolymers are polymeric materials in which two or more monomer sub-units that are polymerized together to create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e. lipophilic), whilst the other sub-unit(s) are hydrophilic whilst in aqueous media. In this case, the block copolymer may possess amphiphilic properties and may form a structure that mimics a biological membrane. The block copolymer may be a diblock (consisting of two monomer sub-units), but may also be constructed from more than two monomer sub-units to form more complex arrangements that behave as amphiphiles. The copolymer may be a triblock, tetrablock or pentablock copolymer. The membrane is preferably a triblock copolymer membrane.

Archaebacterial bipolar tetraether lipids are naturally occurring lipids that are constructed such that the lipid forms a monolayer membrane. These lipids are generally found in extremophiles that survive in harsh biological environments, thermophiles, halophiles and acidophiles. Their stability is believed to derive from the fused nature of the final bilayer. It is straightforward to construct block copolymer materials that mimic these biological entities by creating a triblock polymer that has the general motif hydrophilic-hydrophobic-hydrophilic. This material may form monomeric membranes that behave similarly to lipid bilayers and encompass a range of phase behaviours from vesicles through to laminar membranes. Membranes formed from these triblock copolymers hold several advantages over biological lipid membranes. Because the triblock copolymer is synthesised, the exact construction can be carefully controlled to provide the correct chain lengths and properties required to form membranes and to interact with pores and other proteins.

Block copolymers may also be constructed from sub-units that are not classed as lipid sub-materials; for example a hydrophobic polymer may be made from siloxane or other non-hydrocarbon based monomers. The hydrophilic sub-section of block copolymer can also possess low protein binding properties, which allows the creation of a membrane that is highly resistant when exposed to raw biological samples. This head group unit may also be derived from non-classical lipid head-groups.

Triblock copolymer membranes also have increased mechanical and environmental stability compared with biological lipid membranes, for example a much higher operational temperature or pH range. The synthetic nature of the block copolymers provides a platform to customise polymer based membranes for a wide range of applications.

In some embodiments, the membrane is one of the membranes disclosed in International Application No. WO2014/064443 or WO2014/064444.

The amphiphilic molecules may be chemically-modified or functionalised to facilitate coupling of the polynucleotide. The amphiphilic layer may be a monolayer or a bilayer. The amphiphilic layer is typically planar. The amphiphilic layer may be curved. The amphiphilic layer may be supported.

Amphiphilic membranes are typically naturally mobile, essentially acting as two dimensional fluids with lipid diffusion rates of approximately 10-8 cm s⁻¹. This means that the pore and coupled polynucleotide can typically move within an amphiphilic membrane.

The membrane may be a lipid bilayer. Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer or a liposome. The lipid bilayer is preferably a planar lipid bilayer. Suitable lipid bilayers are disclosed in WO 2008/102121, WO 2009/077734 and WO 2006/100484.

Methods for forming lipid bilayers are known in the art. Lipid bilayers are commonly formed by the method of Montal and Mueller (Proc. Natl. Acad. Sci. USA., 1972; 69:3561-3566), in which a lipid monolayer is carried on aqueous solution/air interface past either side of an aperture which is perpendicular to that interface. The lipid is normally added to the surface of an aqueous electrolyte solution by first dissolving it in an organic solvent and then allowing a drop of the solvent to evaporate on the surface of the aqueous solution on either side of the aperture. Once the organic solvent has evaporated, the solution/air interfaces on either side of the aperture are physically moved up and down past the aperture until a bilayer is formed. Planar lipid bilayers may be formed across an aperture in a membrane or across an opening into a recess.

The method of Montal & Mueller is popular because it is a cost-effective and relatively straightforward method of forming good quality lipid bilayers that are suitable for protein pore insertion. Other common methods of bilayer formation include tip-dipping, painting bilayers and patch-clamping of liposome bilayers.

Tip-dipping bilayer formation entails touching the aperture surface (for example, a pipette tip) onto the surface of a test solution that is carrying a monolayer of lipid. Again, the lipid monolayer is first generated at the solution/air interface by allowing a drop of lipid dissolved in organic solvent to evaporate at the solution surface. The bilayer is then formed by the Langmuir-Schaefer process and requires mechanical automation to move the aperture relative to the solution surface.

For painted bilayers, a drop of lipid dissolved in organic solvent is applied directly to the aperture, which is submerged in an aqueous test solution. The lipid solution is spread thinly over the aperture using a paintbrush or an equivalent. Thinning of the solvent results in formation of a lipid bilayer. However, complete removal of the solvent from the bilayer is difficult and consequently the bilayer formed by this method is less stable and more prone to noise during electrochemical measurement.

Patch-clamping is commonly used in the study of biological cell membranes. The cell membrane is clamped to the end of a pipette by suction and a patch of the membrane becomes attached over the aperture. The method has been adapted for producing lipid bilayers by clamping liposomes which then burst to leave a lipid bilayer sealing over the aperture of the pipette. The method requires stable, giant and unilamellar liposomes and the fabrication of small apertures in materials having a glass surface.

Liposomes can be formed by sonication, extrusion or the Mozafari method (Colas et al. (2007) Micron 38:841-847).

In some embodiments, a lipid bilayer is formed as described in International Application No. WO 2009/077734. Advantageously in this method, the lipid bilayer is formed from dried lipids. In a most preferred embodiment, the lipid bilayer is formed across an opening as described in WO2009/077734.

A lipid bilayer is formed from two opposing layers of lipids. The two layers of lipids are arranged such that their hydrophobic tail groups face towards each other to form a hydrophobic interior. The hydrophilic head groups of the lipids face outwards towards the aqueous environment on each side of the bilayer. The bilayer may be present in a number of lipid phases including, but not limited to, the liquid disordered phase (fluid lamellar), liquid ordered phase, solid ordered phase (lamellar gel phase, interdigitated gel phase) and planar bilayer crystals (lamellar sub-gel phase, lamellar crystalline phase).

Any lipid composition that forms a lipid bilayer may be used. The lipid composition is chosen such that a lipid bilayer having the required properties, such surface charge, ability to support membrane proteins, packing density or mechanical properties, is formed. The lipid composition can comprise one or more different lipids. For instance, the lipid composition can contain up to 100 lipids. The lipid composition preferably contains 1 to 10 lipids. The lipid composition may comprise naturally-occurring lipids and/or artificial lipids.

The lipids typically comprise a head group, an interfacial moiety and two hydrophobic tail groups which may be the same or different. Suitable head groups include, but are not limited to, neutral head groups, such as diacylglycerides (DG) and ceramides (CM); zwitterionic head groups, such as phosphatidylcholine (PC), phosphatidylethanolamine (PE) and sphingomyelin (SM); negatively charged head groups, such as phosphatidylglycerol (PG); phosphatidylserine (PS), phosphatidylinositol (PI), phosphatic acid (PA) and cardiolipin (CA); and positively charged headgroups, such as trimethylammonium-Propane (TAP). Suitable interfacial moieties include, but are not limited to, naturally-occurring interfacial moieties, such as glycerol-based or ceramide-based moieties. Suitable hydrophobic tail groups include, but are not limited to, saturated hydrocarbon chains, such as lauric acid (n-Dodecanolic acid), myristic acid (n-Tetradecononic acid), palmitic acid (n-Hexadecanoic acid), stearic acid (n-Octadecanoic) and arachidic (n-Eicosanoic); unsaturated hydrocarbon chains, such as oleic acid (cis-9-Octadecanoic); and branched hydrocarbon chains, such as phytanoyl. The length of the chain and the position and number of the double bonds in the unsaturated hydrocarbon chains can vary. The length of the chains and the position and number of the branches, such as methyl groups, in the branched hydrocarbon chains can vary. The hydrophobic tail groups can be linked to the interfacial moiety as an ether or an ester. The lipids may be mycolic acid.

The lipids can also be chemically-modified. The head group or the tail group of the lipids may be chemically-modified. Suitable lipids whose head groups have been chemically-modified include, but are not limited to, PEG-modified lipids, such as 1,2-Diacyl-sn-Glycero-3-Phosphoethanolamine-N-[Methoxy (Polyethylene glycol)-2000]; functionalised PEG Lipids, such as 1,2-Distearoyl-sn-Glycero-3 Phosphoethanolamine-N-[Biotinyl (Polyethylene Glycol) 2000]; and lipids modified for conjugation, such as 1,2-Dioleoyl-sn-Glycero-3-Phosphoethanolamine-N-(succinyl) and 1,2-Dipalmitoyl-sn-Glycero-3-Phosphoethanolamine-N-(Biotinyl). Suitable lipids whose tail groups have been chemically-modified include, but are not limited to, polymerisable lipids, such as 1,2-bis (10,12-tricosadiynoyl)-sn-Glycero-3-Phosphocholine; fluorinated lipids, such as 1-Palmitoyl-2-(16-Fluoropalmitoyl)-sn-Glycero-3-Phosphocholine; deuterated lipids, such as 1,2-Dipalmitoyl-D62-sn-Glycero-3-Phosphocholine; and ether linked lipids, such as 1,2-Di-O-phytanyl-sn-Glycero-3-Phosphocholine. The lipids may be chemically-modified or functionalised to facilitate coupling of the polynucleotide.

The amphiphilic layer, for example the lipid composition, typically comprises one or more additives that will affect the properties of the layer. Suitable additives include, but are not limited to, fatty acids, such as palmitic acid, myristic acid and oleic acid; fatty alcohols, such as palmitic alcohol, myristic alcohol and oleic alcohol; sterols, such as cholesterol, ergosterol, lanosterol, sitosterol and stigmasterol; lysophospholipids, such as 1-Acyl-2-Hydroxy-sn-Glycero-3-Phosphocholine; and ceramides.

In another embodiment, the membrane comprises a solid state layer. Solid state layers can be formed from both organic and inorganic materials including, but not limited to, microelectronic materials, insulating materials such as Si₃N₄, Al₂O₃, and SiO, organic and inorganic polymers such as polyamide, plastics such as Teflon® or elastomers such as two-component addition-cure silicone rubber, and glasses. The solid state layer may be formed from graphene. Suitable graphene layers are disclosed in WO 2009/035647. If the membrane comprises a solid state layer, the pore is typically present in an amphiphilic membrane or layer contained within the solid state layer, for instance within a hole, well, gap, channel, trench or slit within the solid state layer. The skilled person can prepare suitable solid state/amphiphilic hybrid systems. Suitable systems are disclosed in WO 2009/020682 and WO 2012/005857. Any of the amphiphilic membranes or layers discussed above may be used.

The methods disclosed herein are typically carried out using (i) an artificial amphiphilic layer comprising a pore, (ii) an isolated, naturally-occurring lipid bilayer comprising a pore, or (iii) a cell having a pore inserted therein. The methods are typically carried out using an artificial amphiphilic layer, such as an artificial triblock copolymer layer. The layer may comprise other transmembrane and/or intramembrane proteins as well as other molecules in addition to the pore. Suitable apparatus and conditions are discussed below. The method of the invention is typically carried out in vitro.

### General Methods

The methods provided herein may be operated using any suitable nanopore, and as such any suitable nanopore apparatus for detecting polynucleotides can be used.

The methods provided herein may in some embodiments be carried out using any apparatus that is suitable for transmembrane pore sensing. For example, the apparatus may comprise a chamber comprising an aqueous solution and a barrier that separates the chamber into two sections. The barrier may have an aperture in which a membrane containing a transmembrane pore is formed. Transmembrane pores are described herein.

The methods may be carried out using the apparatus described in WO 2008/102120, WO 2010/122293 or WO 00/28312. In brief, the binding of a molecule (e.g. a target polynucleotide) in the channel of a pore will have an effect on the open-channel ion flow through the pore, which is the essence of “molecular sensing” of pore channels. Variation in the open-channel ion flow can be measured using suitable measurement techniques by the change in electrical current. The degree of reduction in ion flow, as measured by the reduction in electrical current, is related to the size of the obstruction within, or in the vicinity of, the pore. Binding of a molecule of interest (e.g. the target polynucleotide) in or near the pore therefore provides a detectable and measurable event, thereby forming the basis of a “biological sensor”. Detecting the presence of biological molecules finds application in personalised drug development, medicine, diagnostics, life science research, environmental monitoring and in the security and/or the defence industry.

When used to characterize the polynucleotide, the methods may involve measuring the ion current flow through the pore, typically by measurement of a current. Alternatively, the ion flow through the pore may be measured optically, such as disclosed by Heron et al: J. Am. Chem. Soc. 9 Vol. 131, No. 5, 2009. Therefore, the apparatus may also comprise an electrical circuit capable of applying a potential and measuring an electrical signal across the membrane and pore. The characterisation methods may be carried out using a patch clamp or a voltage clamp. The characterisation methods preferably involve the use of a voltage clamp.

The methods may involve measuring an optical signal as described in Chen et al, Nature Communications (2018) 9:1733, the entire contents of which are hereby incorporated by reference. For example, a nanopore such as an optically engineered nanopore structure (e.g. a plasmonic nanoslit) may be used to locally enable single-molecule surface enhanced Raman spectroscopy (SERS) to allow the characterisation of the polynucleotide through direct Raman spectroscopic detection.

The methods may be carried out on a silicon-based array of wells where each array comprises 128, 256, 512, 1024, 2000, 3000, 4000, 6000, 10000, 12000, 15000 or more wells.

The methods may involve the measuring of a current flowing through the pore. The method is typically carried out with a voltage applied across the membrane and pore. The voltage used is typically from +2 V to −2 V, typically −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 100 mV to 240 mV and most preferably in the range of 120 mV to 220 mV. It is possible to increase discrimination between different nucleotides by a pore by using an increased applied potential.

The voltage is applied across the nanopore via one or more electrodes. In some embodiments, the voltage is applied across the nanopore via one or more platinum electrodes. In some embodiments the voltage is applied across the nanopore via one or more platinum electrodes and the conditions which decrease formation of cation-mediated secondary structure comprise the use of sodium chloride and sodium ferricyanide/sodium ferrocyanide salts. Surprisingly, such conditions are compatible.

With reference to the conditions desired herein, the methods are typically carried out in the presence of any charge carriers, such as metal salts, for example alkali metal salts, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In the exemplary apparatus discussed above, the salt is present in the aqueous solution in the chamber. The salts typically used as charge carriers are described herein and above.

The methods are typically carried out in the presence of a buffer. In the exemplary apparatus discussed above, the buffer is present in the aqueous solution in the chamber. Any suitable buffer may be used. Typically, the buffer is HEPES. Another suitable buffer is Tris-HCl buffer. The methods are typically carried out at a pH of from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 7.5.

### Further Aspects

Also provided herein as methods of operating a nanopore. In some embodiments the methods comprise providing a condition which decreases formation of cation-mediated secondary structure. In some embodiments the nanopore is disposed in said condition. In some embodiments, the method involves operating the nanopore by translocating a portion of a polynucleotide through the nanopore. In some embodiments the method involves operating the nanopore by translocating a portion of a polynucleotide through the nanopore under conditions such that blocking of the nanopore is inhibited. In some embodiments the nanopore is present in a nanopore array. In some embodiments the nanopore array comprises a plurality of nanopores.

In some embodiments, provided herein are methods of operating a nanopore array. In some embodiments the methods comprise providing a condition which decreases formation of cation-mediated secondary structure in polynucleotides that may be contacted with the array. In some embodiments, the method involves operating the array by contacting a plurality of polynucleotides with the array and translocating a portion of each polynucleotide through the array. In some embodiments the method involves operating the array by translocating a portion of a polynucleotide through the nanopore under conditions such that blocking of the nanopores in the array is inhibited. In some embodiments the methods involve increasing the lifetime of the array. In some embodiments the or each nanopore is a nanopore as described in more detail herein. In some embodiments the polynucleotide is a polynucleotide as described herein. In some embodiments the conditions are as described herein.

Also provided herein are methods of unblocking a nanopore, such as a nanopore in a nanopore array. In some embodiments the methods are methods of unblocking a nanopore which is blocked by a polynucleotide. In some embodiments the methods comprise contacting the nanopore with providing a condition which decreases formation of cation-mediated secondary structure, such as a condition as described herein.

In some embodiments, the methods described herein are methods of increasing the usable lifetime of a nanopore. In some embodiments, the methods described herein methods of increasing the lifetime of a nanopore array. In some embodiments the lifetime of the nanopore or nanopore array is increased by reducing the blocking of the nanopore or of the nanopores in the array. In some embodiments the lifetime of the nanopore or array is increased by contacting the nanopore or array with a condition which decreases formation of cation-mediated secondary structure, such as a condition as described herein

In some embodiments, the methods described herein are methods of increasing the efficiency of polynucleotide characterisation by a nanopore or a nanopore array. In some embodiments, the methods described herein are methods of increasing the efficiency of the operation of a nanopore or a nanopore array. In some embodiments the methods involve increasing the efficiency of polynucleotide characterisation by decreasing blocking of the nanopore or of nanopores in the array. In some embodiments the efficiency of characterisation is increased by contacting the nanopore or array with a condition which decreases formation of cation-mediated secondary structure, such as a condition as described herein

In some of the aforementioned embodiments, the or each nanopore may be a nanopore as described in more detail herein. In some embodiments the or each polynucleotide is a polynucleotide as described herein. In some embodiments the conditions are as described herein.

### Kits

Also described herein is a kit comprising (a) a nanopore in a membrane and (b) one or more conditions as described herein, such as a solution comprising one or more conditions as described herein. Also provided is a kit comprising (a) a nanopore in a membrane and (b) a solution comprising a sodium ferricyanide/sodium ferrocyanide salt. Further provided is a kit comprising (a) a nanopore in a membrane and (b) a solution comprising one or more metal salts selected from the group consisting of one or more rubidium salts, sodium ferricyanide and sodium ferrocyanide. In some embodiments, the one or more metal salts are selected from the group consisting of (i) rubidium ferricyanide and/or rubidium ferrocyanide, (ii) rubidium chloride, and (iii) sodium ferricyanide and sodium ferrocyanide. In some embodiments, the one or more conditions comprise a salt comprising an ammonium cation and/or a metal salt as described herein. In some embodiments, the salt comprising an ammonium cation comprises (i) ammonium ferricyanide and/or ammonium ferrocyanide, and/or (ii) ammonium chloride. In some embodiments, the rubidium salt is rubidium chloride. It will be understood that any of the conditions, nanopores, polynucleotide-handling enzymes, anchors and adaptors can be applied to the kit discussed herein and above.

In some embodiments, the kit further comprises a charge carrier as described herein. For example, in some embodiments the charge carrier is a further metal salt. Typically, the further metal salt is not a potassium salt. In some embodiments, the further metal salt comprises a cation selected from the group consisting of Na⁺, Cs⁺, Li⁺, Mg²⁺ and Ca²⁺, typically Na⁺. In some embodiments, the further metal salt comprises a cation selected from the group consisting of Rb⁺, Na⁺, Cs⁺, Li⁺, Mg²⁺ and Ca²⁺, typically Rb⁺ or Na⁺. In some embodiments, the further metal salt is a chloride salt. In some embodiments, the further metal salt is a chloride salt comprising a cation selected from the group consisting of Na⁺, Cs⁺, Li⁺, Mg²⁺ and Ca²⁺, typically Na⁺. In some embodiments, the further metal salt is a chloride salt comprising a cation selected from the group consisting of Rb⁺, Na⁺, Cs⁺, Lit, Mg²⁺ and Ca²⁺, typically Rb⁺ or Na⁺. In some embodiments the further metal salt is RbCl. In some embodiments the further metal salt is NaCl. In embodiments where the further metal salt is not present on the trans side of a nanopore in the membrane, the further metal salt may comprise a potassium salt, such as KCl.

In some embodiments, the kit further comprises a polynucleotide-handling enzyme as described herein. In some embodiments, the kit further comprises an anchor as described herein. In some embodiments, the kit further comprises an adaptor as described herein. In some embodiments, the nanopore in the kit may be comprised in one or more independently addressable elements as further described herein, wherein each independently addressable element comprises a cis and a trans chamber connected by an electrical communication means, wherein said cis and trans chambers are separated by the membrane comprising the nanopore.

### Apparatuses

Also described herein is an apparatus comprising (a) an array of nanopores in a membrane and (b) one or more conditions, as described herein, in contact with the membrane. Also provided is an apparatus comprising (a) an array of nanopores in a membrane and (b) a sodium ferricyanide/sodium ferrocyanide salt solution in contact with the membrane. Further provided is an apparatus comprising (a) an array of nanopores in a membrane and (b) a solution comprising one or more metal salts in contact with the membrane, wherein the one or more metal salts are selected from the group consisting of one or more rubidium salts, sodium ferricyanide and sodium ferrocyanide. In some embodiments, the one or more metal salts are selected from the group consisting of (i) rubidium ferricyanide and/or rubidium ferrocyanide, (ii) rubidium chloride, and (iii) sodium ferricyanide and sodium ferrocyanide. In some embodiments, the one or more conditions comprise a salt comprising an ammonium cation and/or a metal salt as described herein. In some embodiments, the salt comprising an ammonium cation comprises (i) ammonium ferricyanide and/or ammonium ferrocyanide, and/or (ii) ammonium chloride. It will be understood that any of the conditions, nanopores, polynucleotide-handling enzymes, anchors and adaptors can be applied to the apparatus discussed herein and above.

In some embodiments, the apparatus further comprises a charge carrier as described herein. For example, in some embodiments the charge carrier is a further metal salt. Typically, the further metal salt is not a potassium salt. In some embodiments, the further metal salt comprises one or more cations selected from the group consisting of Rb⁺, Na⁺, Cs⁺, Li⁺, Mg²⁺ and Ca²⁺, typically Rb⁺ or Na⁺. In some embodiments, the further metal salt comprises a cation selected from the group consisting of Na⁺, Cs⁺, Li⁺, Mg²⁺ and Ca²⁺, typically Na⁺. In some embodiments, the further metal salt comprises one or more cations selected from the group consisting of Rb⁺, Na⁺, Cs⁺, Lit, and Ca²⁺. In some embodiments, the further metal salt comprises one or more cations selected from the group consisting of Rb⁺, Na⁺, Cs⁺ and Lit, and Ca²⁺. In some embodiments, the further metal salt is a chloride salt. In some embodiments, the further metal salt comprises a chloride salt comprising one or more cations selected from the group consisting of Rb⁺, Na⁺, Cs⁺, Li⁺, Mg²⁺ and Ca²⁺, typically Rb⁺ or Na⁺. In some embodiments, the further metal salt is a chloride salt comprising a cation selected from the group consisting of Na⁺, Cs⁺, Li⁺, Mg²⁺ and Ca²⁺, typically Na⁺. In some embodiments, the further metal salt comprises a chloride salt comprising one or more cations selected from the group consisting of Rb⁺, Na⁺, Cs⁺, Li⁺ and Ca²⁺. In some embodiments, the further metal salt is a chloride salt comprising one or more cations selected from the group consisting of Rb⁺, Na⁺, Cs⁺ and Lit. In some embodiments the further metal salt is RbCl. In some embodiments the further metal salt is NaCl.

In some embodiments, the apparatus further comprises a polynucleotide-handling enzyme as described herein. In some embodiments, the apparatus further comprises an anchor as described herein. In some embodiments, the apparatus further comprises an adaptor as described herein.

In some embodiments, the array of nanopores in a membrane may be an array of individually addressable elements, wherein each independently addressable element of the array comprises a cis and a trans chamber connected by an electrical communication means, wherein said cis and trans chambers are separated by the membrane, and the membrane of each independently addressable element comprises a single nanopore. In some embodiments, the cis and the trans chamber each comprise the solution. In some embodiments, the trans chamber is substantially free of a potassium cation. In some embodiments, the trans chamber and the cis chamber is substantially free of a potassium cation. In some embodiments, the trans chamber comprises the solution, and the cis chamber comprises a different solution comprising one or more metal salts. In this embodiment, the cis chamber may or may not comprise conditions which decrease the formation of cation-mediated secondary structure. For example, the cis chamber may comprise a metal salt comprising a potassium cation. In some embodiments, the apparatus comprises a polynucleotide handling enzyme on the cis side of the membrane. In some embodiments, the independently addressable elements of the array share a common cis chamber. In some embodiments, each independently addressable element has a trans chamber separate from the trans chambers of other independently addressable elements of the array. In some embodiments, the apparatus comprises a voltage source connected to electrodes, wherein the voltage source and electrodes are configured to apply an electric field between the cis and the trans chambers. In some embodiments, the apparatus comprises a digital logic circuit associated with the nanopores of the independently addressable elements of the array of independently addressable elements. Such configurations of the apparatus are described in more detail in WO 2008/124107 A1.

In some embodiments, the independently addressable elements of the array share a common cis electrode. In some embodiments, each independently addressable element has a trans electrode separate from the trans electrode of other independently addressable elements of the array. In some embodiments, the apparatus comprises a cis buffer in contact with the cis electrode. In some embodiments, the cis buffer comprises a metal salt, such as a metal salt comprising one or more cations selected from the group consisting of K⁺, Rb⁺, Na⁺, Cs⁺, Li⁺, Mg²⁺ and Ca²⁺, typically K⁺, Rb⁺ or Na⁺. In some embodiments, the trans buffer is substantially free of a potassium cation. In some embodiments, the trans buffer comprises a metal salt comprising one or more cations selected from the group consisting of Rb⁺, Na⁺, Cs⁺, Li⁺, Mg²⁺ and Ca²⁺, typically Rb⁺ or Na⁺.

The kit and apparatus disclosed herein may be configured for use with an algorithm, also provided herein, adapted to be run on a computer system. The algorithm may be adapted to detect information characteristic of a polynucleotide (e.g. characteristic of the sequence of the polynucleotide), and to selectively process the signal obtained as the polynucleotide moves with respect to a nanopore. Also provided is an apparatus comprising computing means configured to detect information characteristic of a polynucleotide (e.g. characteristic of the sequence of the polynucleotide) and to selectively process the signal obtained as the polynucleotide moves with respect to the nanopore. In some embodiments the system comprises receiving means for receiving data from detection of the polynucleotide, processing means for processing the signal obtained as the polynucleotide moves with respect to the nanopore, and output means for outputting the characterisation information thus obtained.

### Uses

Also provided herein is use of a sodium salt for reducing cation-mediated secondary structure in a polynucleotide in a nanopore apparatus or during a method of characterising a polynucleotide using a nanopore. Further provided herein is use of one or more metal salts for reducing cation-mediated secondary structure in a polynucleotide in a nanopore apparatus or during a method of characterising a polynucleotide using a nanopore, wherein the one or more metal salts are selected from the group consisting of one or more rubidium salts, sodium ferricyanide and sodium ferrocyanide. It will be understood that any of the methods, conditions, nanopores, polynucleotide-handling enzymes, anchors, adaptors and apparatuses can be applied to the use discussed herein and above.

In some embodiments, the sodium salt is a sodium ferricyanide/sodium ferrocyanide salt solution. In some embodiments, the sodium salt further comprises a charge carrier as described herein. In some embodiments, the one or more metal salts are selected from the group consisting of (i) rubidium ferricyanide and/or rubidium ferrocyanide, (ii) rubidium chloride, and (iii) sodium ferricyanide and sodium ferrocyanide. In some embodiments, the rubidium salt further comprises a charge carrier as described herein. For example, in some embodiments, the further metal salt comprises one or more cations selected from the group consisting of Rb⁺, Na⁺, Cs⁺, Li⁺, Mg²⁺ and Ca²⁺, typically Rb⁺ or Na⁺. In some embodiments, the further metal salt comprises one or more cations selected from the group consisting of Rb⁺, Na⁺, Cs⁺, Lit and Ca²⁺. In some embodiments, the further metal salt comprises one or more cations selected from the group consisting of Rb⁺, Na⁺, Cs⁺ and Lit. For example, in some embodiments the charge carrier is a further metal salt. Typically, the further metal salt is not a potassium salt. In some embodiments, the further metal salt comprises a cation selected from the group consisting of Na⁺, Cs⁺, Li⁺, Mg²⁺ and Ca²⁺, typically Na⁺. In some embodiments, the further metal salt is a chloride salt. In some embodiments, the further metal salt comprises a chloride salt comprising one or more cations selected from the group consisting of Rb⁺, Na⁺, Cs⁺, Li⁺, Mg²⁺ and Ca²⁺, typically Rb⁺ or Na⁺. In some embodiments, the further metal salt is a chloride salt comprising a cation selected from the group consisting of Na⁺, Cs⁺, Li⁺, Mg²⁺ and Ca²⁺, typically Na⁺. In some embodiments, the further metal salt comprises a chloride salt comprising one or more cations selected from the group consisting of Rb⁺, Na⁺, Cs⁺, Lit and Ca²⁺. In some embodiments, the further metal salt comprises a chloride salt comprising one or more cations selected from the group consisting of Rb⁺, Na⁺, Cs⁺ and Lit. In some embodiments the further metal salt is RbCl. In some embodiments the further metal salt is NaCl.

In some embodiments, the sodium salt is used in any of the methods described herein. In some embodiments, the sodium salt is used in any of the apparatuses described herein. In some embodiments, the rubidium salt is used in any of the methods described herein. In some embodiments, the rubidium salt is used in any of the apparatuses described herein.

It is to be understood that although particular embodiments, specific configurations as well as materials and/or molecules, have been discussed herein for methods according to the present invention, various changes or modifications in form and detail may be made without departing from the scope and spirit of this invention. The preceding embodiments and following examples are provided for illustration only, and should not be considered limiting the application. The application is limited only by the claims.

The following examples illustrate the invention.

### EXAMPLES

### Example 1

A sample comprising human genomic DNA 30 kb in length was prepared and added to flow cells in a nanopore sequencing device from Oxford Nanopore Technologies, each flow cell comprising an array of transmembrane protein nanopores such as described herein, under control conditions and under conditions as disclosed herein. The control conditions comprise potassium chloride, potassium ferrocyanide and potassium ferricyanide salts on both sides of the membrane. The conditions as disclosed herein comprise a sodium salt on the cis and trans side of the membrane.

Electrical measurements were acquired and the total read output was plotted over time comparing the control conditions to the conditions comprising the sodium salt. As can be seen the results showed that the conditions comprising the sodium salt resulted in a greater than 30% increase in the total read output over the measurement period (FIG. 2).

The experiment was repeated using a polynucleotide comprising the G-quadruplex structure from thrombin-binding aptamer. As shown in FIG. 3, where a sodium salt was added to the cis side of the membrane, and a potassium salt was present on the trans side of the membrane, the total output was the same as compared to control conditions. However, when the sodium salt was added to both the trans and cis sides of the membrane a greater than 70% increase in the total output was achieved when compared to control conditions.

In order to characterise the extent to which sodium may decrease the formation of G-quadruplex secondary structure when compared to conditions comprising potassium salts, the melting temperature of the G-quadruplex from the thrombin-binding aptamer in the presence of different salts was determined by fluorescence measurements. The 3′ and 5′ ends of a polynucleotide comprising a G-quadruplex structure were attached to a quencher and a fluorophore such that the fluorescence was quenched when the G-quadruplex was formed around the cation. Upon denaturing of the G-quadruplex, the quenching of the fluorophore was reduced (FIG. 4). It was shown that in the presence of a potassium salt the melting temperature of the G-quadruplex was approximately 65° C., but in the presence of a sodium salt the melting temperature of the G-quadruplex was approximately 50° C., significantly higher than the temperature at which the electrical recordings in FIGS. 2 and 3 were obtained.

### Example 2

A further experiment to characterise the extent to which alternative ions to potassium ions decrease the formation of G-quadruplex secondary structure was carried out, in which the melting temperature of the G-quadruplex from the thrombin-binding aptamer in the presence of different salts was determined by fluorescence measurements. The following salt concentrations were used in this experiment:
K: 150 mM K Ferrocyanide, 150 mM K Ferricyanide, 25 mM K phosphate monobasic, pH 8.
Rb: 150 mM Rb Ferrocyanide, 150 mM Rb Ferricyanide, 25 mM Na phosphate monobasic, pH 7.
Na: 210 mM Na Ferrocyanide, 210 mM Na Ferricyanide, 25 mM Na phosphate monobasic, pH 7.
Na 150:150 mM Na Ferrocyanide, 150 mM Na Ferricyanide, 25 mM Na phosphate monobasic, pH 7.

The 3′ and 5′ ends of a polynucleotide comprising a G-quadruplex structure were attached to a quencher and a fluorophore such that the fluorescence was quenched when the G-quadruplex was formed around a cation. Raising the temperature results in denaturing of the G-quadruplex; the quenching of the fluorophore is then reduced (FIG. 5). The melting temperature of the G-quadruplex in the presence of potassium ions under the above conditions was determined to be approximately 75° C. In the presence of sodium ions the melting temperature of the G-quadruplex was determined to be approximately 65° C. The melting temperature of the G-quadruplex was also shown to be concentration dependent with lower concentrations of cation leading to small variations in melting temperature (smaller than the ion to ion differences). Repeating the fluorescence assay in the presence of rubidium ions further lowered the melting temperature of the G-quadruplex to 55° C.

### Example 3

A sample comprising human genomic DNA was extracted from HG002 cells (Coriell) using a Puregene extraction kit (Qiagen), sheared using a Megaruptor (Diagenode), and size selected using a Pippin HT (Sage Science) to deplete the short fragment content and obtain a final library with a read length N50 of 30 kilobases. This DNA sample was prepared for nanopore sequencing using Oxford Nanopore Technologies ligation sequencing kit SQK-LSK114. Following this preparation step, 500 ng of the sample was added to Oxford Nanopore Technologies PromethION R10 flow cells, each flow cell comprising an array of transmembrane protein nanopores, under control and test conditions. The control conditions comprised potassium chloride on the cis side of the membrane, and potassium ferrocyanide and potassium ferricyanide salts on the trans side of the membrane. The test conditions comprised either rubidium or sodium salts on the trans side of the membrane with potassium chloride salts on the cis side of the membrane. The following salt concentrations were used for the sodium and rubidium test conditions, respectively:
Sodium salt: 210 mM Na Ferrocyanide, 210 mM Na Ferricyanide, 25 mM Na phosphate monobasic, pH 7.
Rubidium salt: 145 mM Rb Ferrocyanide, 145 mM Rb Ferricyanide, 25 mM Na phosphate monobasic, pH 7.

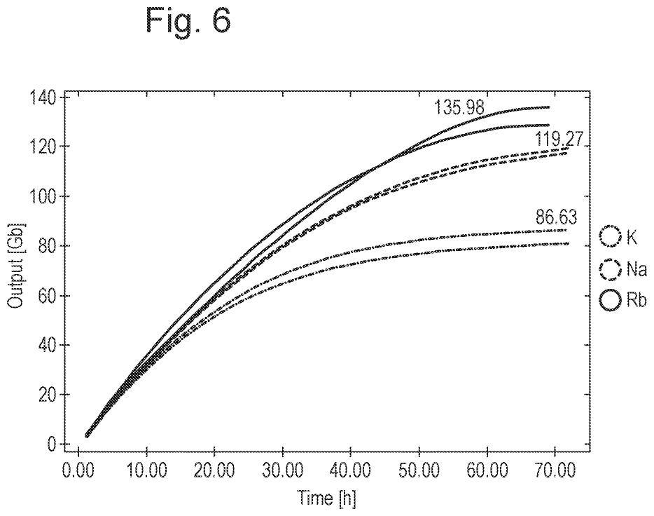

Electrical measurements were acquired by running a standard sequencing script on MinKNOW software version 5.9, and the total read output was plotted over time comparing the control conditions (potassium salts) to the test conditions comprising sodium or rubidium salts on the trans side of the membrane. The results showed that the presence of the sodium salt (or rubidium salt) on the trans side of the membrane resulted in a greater than 37% (55% for rubidium salt) increase in the total read output over the measurement period (FIG. 6). This also demonstrated that the trend in G-quadruplex stability described in Example 2 above also holds for increases in sequencing output during nanopore sequencing, despite the sequencing temperature being lower than the measured melting temperature as described above.

### Example 4

The experiment described above in Example 3 was repeated using similarly extracted HG002 genomic DNA, this time sheared more intensely down to a read length N50 of 15 kilobases and prepared using Oxford Nanopore Technologies native barcoding kit (SQK-NBD114.24) for multiplexing samples. 500 ng of this DNA library was added to PromethION R10 flow cells and run on MinKNOW version 5.9 with a standard sequencing script. Control flow cells were paused after 22 hours and washed with Oxford Nanopore Technologies wash kit (EXP-WSH004) before being reloaded with another 500 ng of the same 15 kilobase library and run for the remainder of the experiment. These control flow cells were compared to flow cells where rubidium salts were present on the trans side of the membrane, where washing and reloading steps were not performed. The EXP-WSH004 wash kit comprises a nuclease to digest and remove nucleic acid on the cis side of the membrane in the flow cell.

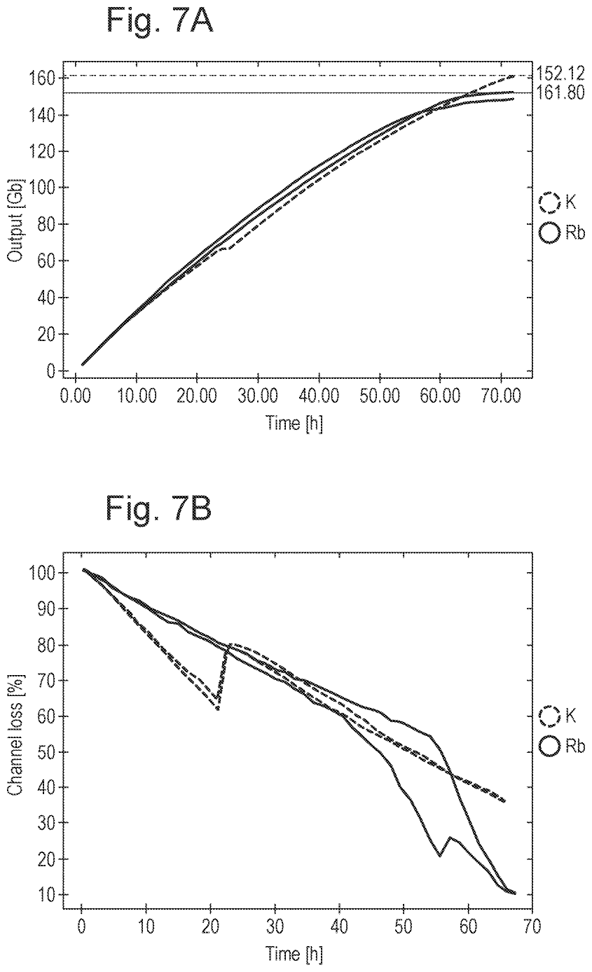

FIG. 7A shows the total read output over time for this experiment, showing that the rubidium condition obtains similar total outputs despite not being washed and reloaded. FIG. 7B shows a graph tracking the channel loss over time for this experiment. The slope of channel loss is representative of the decrease of available nanopore sequencing channels/throughput over time. The control traces show clear recovery in the number of channels available at 22 hours after the wash protocol was carried out on the control flow cells. The rubidium conditions show that the slope of channel loss is much shallower-believed to be due to the destabilisation of G-quadruplex structures in the trans chamber that would otherwise block channels-leading to longer retention of throughput. The decrease in channel loss demonstrates that the presence of rubidium ions in the trans chamber enables the rubidium-containing flow cells to achieve a similar total sequencing output compared to the control flow cells, without the need for either the addition of more DNA library or for user interaction with the flow cell such as the use of a nuclease wash step.

### Example 5

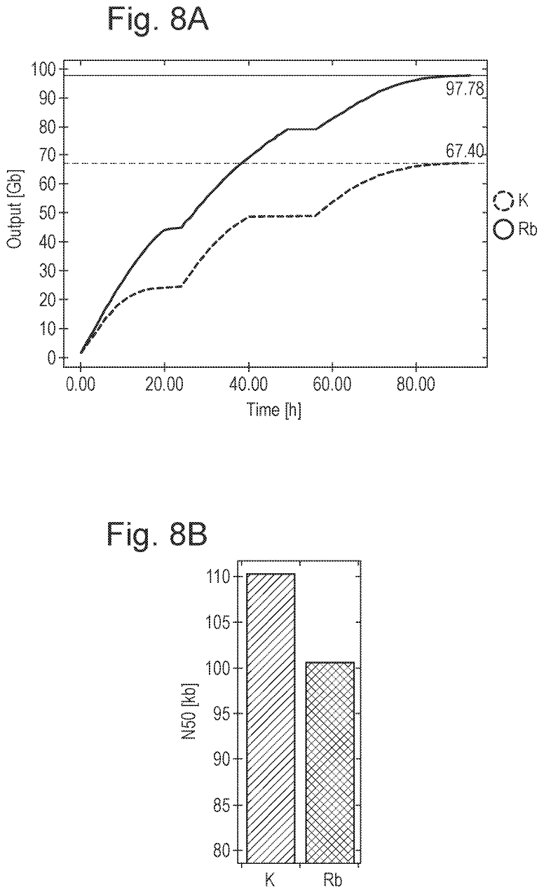

A further experiment was carried out using human genomic DNA extracted from HG002 cells (Coriell) using Monarch high molecular weight extraction kit (NEB) and prepared using Oxford Nanopore Technologies ultra-long sequencing kit (SQK-ULK114). These libraries were similarly loaded onto PromethION R10 flow cells (as described above with regard to Examples 3 and 4) and run on MinKNOW version 5.9. The ultra-long kit produces libraries with very long DNA fragments; in this case the read length N50 of the libraries was over 100 kilobases (FIG. 8B). These long DNA fragments typically result in rapid loss of channels on a flow cell. As such all flow cells were washed with Oxford Nanopore Technologies wash kit (EXP-WSH004) when channel loss reached ~70% of starting channels. This was repeated twice during the 72-hour run for a total of two washes and three loads. FIG. 8A shows the total read output for this experiment comparing control flow cells to flow cells with rubidium salts in the trans chamber. The rubidium condition shows an increase in total output of 46%. This experiment demonstrates that the rubidium-containing flow cells were not adversely affected by the use of a nuclease wash step.

### DESCRIPTION OF THE SEQUENCE LISTING

SEQ ID NO: 1 shows the amino acid sequence of (hexa-histidine tagged) exonuclease I (EcoExo I) from E. coli.

SEQ ID NO: 2 shows the amino acid sequence of the exonuclease III enzyme from E. coli. SEQ ID NO: 3 shows the amino acid sequence of the RecJ enzyme from T. thermophilus (TthRecJ-cd).

SEQ ID NO: 4 shows the amino acid sequence of bacteriophage lambda exonuclease. The sequence is one of three identical subunits that assemble into a trimer. (http://www.neb.com/nebecomm/products/productM0262.asp).

SEQ ID NO: 5 shows the amino acid sequence of Phi29 DNA polymerase from Bacillus subtilis phage Phi29.

SEQ ID NO: 6 shows the amino acid sequence of Trwc Cba (Citromicrobium bathyomarinum) helicase.

SEQ ID NO: 7 shows the amino acid sequence of Hel308 Mbu (Methanococcoides burtonii) helicase.

SEQ ID NO: 8 shows the amino acid sequence of the Dda helicase 1993 from Enterobacteria phage T4.

SEQ ID NO: 9 shows the nucleotide sequence of thrombin binding aptamer

### FURTHER EMBODIMENTS

1. A method of characterising a polynucleotide, comprising:
   (a) contacting the polynucleotide with a nanopore having a first opening and a second opening;
   (b) translocating a portion of the polynucleotide through the nanopore in a first direction with respect to the nanopore, wherein the first direction is from the first opening to the second opening;
   (c) contacting the translocated portion of the polynucleotide with one or more conditions which decrease formation of cation-mediated secondary structure;
   (d) translocating the polynucleotide in a second direction with respect to the nanopore, wherein the second direction is from the second opening to the first opening; and
   (e) taking one or more measurements characteristic of the polynucleotide during the translocation of the polynucleotide through the nanopore, thereby characterising the polynucleotide.
2. A method according to embodiment 1, wherein if the measurements characteristic of the polynucleotide are indicative of one or more desired characteristics of the polynucleotide, the method comprises translocating the entire polynucleotide through the nanopore and taking one or more measurements during said translocation.
3. A method according to embodiment 1 or embodiment 2, comprising repeating steps (b) to (d) multiple times.
4. A method according to embodiment 1, wherein if the measurements characteristic of the polynucleotide are indicative of one or more undesired characteristics of the polynucleotide, the method comprises ejecting the polynucleotide from the first opening of the nanopore.
5. A method according to embodiment 4, wherein the one or more undesired characteristics include the inability of the polynucleotide to fully translocate through the nanopore in the first direction with respect to the nanopore.
6. A method according to any one of the preceding embodiments, wherein the method comprises characterising a plurality of polynucleotides using a plurality of nanopores, optionally wherein said plurality of nanopores are present in a nanopore array.
7. A method of operating a nanopore array, wherein each nanopore in the array has a first opening and a second opening, the method comprising:
   (a) contacting the array with a plurality of polynucleotides under conditions such that a portion of each polynucleotide translocates through the array in a first direction with respect to the nanopores in the array, wherein the first direction is from the first opening to the second opening;
   (b) contacting the translocated portion of the polynucleotides with one or more conditions which decrease formation of cation-mediated secondary structure;
   (c) determining whether each polynucleotide translocated in step (b) has a desired characteristic; and
   (d) ejecting polynucleotides which do not have the desired characteristic by translocating the polynucleotide in a second direction with respect to the nanopores in the array, wherein the second direction is from the second opening to the first opening.
8. A method according to embodiment 7, comprising taking one or more measurements characteristic of the polynucleotides in the plurality of polynucleotides during the translocation of the portion of each polynucleotide through the nanopore, thereby characterising the polynucleotides.
9. A method according to any one of the preceding embodiments, wherein the method is performed at a temperature lower than the melting temperature of the cation-mediated secondary structure.
10. A method according to any one of the preceding embodiments, wherein the cation-mediated secondary structure is a guanine tetrad or a G-quadruplex, preferably a G-quadruplex.
11. A method according to any one of the preceding embodiments, wherein the one or more conditions comprise a metal salt that disrupts the stability of the cation-mediated secondary structure.
12. A method according to embodiment 11, wherein the metal salt comprises a cation selected from the group consisting of Na⁺, Cs⁺, Li⁺, Mg²⁺ and Ca²⁺.
13. A method according to embodiment 11 or 12, wherein the metal salt comprises a Na⁺ cation.
14. A method according to any one of embodiments 11 to 13, wherein the metal salt comprises sodium ferricyanide and/or sodium ferrocyanide.
15. A method according to any one of the preceding embodiments, wherein the conditions are substantially free of K⁺.
16. A method according to any one of the preceding embodiments, wherein the polynucleotide, or each polynucleotide in the plurality of the polynucleotides, has a length of 100 nucleotides or more.
17. A method according to any one of the preceding embodiments, comprising controlling the movement of the or each polynucleotide with respect to the nanopore in the first direction using a polynucleotide-handling protein.
18. A method according to embodiment 17, wherein the polynucleotide-handling protein is or comprises a helicase, translocase or helicase-nuclease complex.
19. A method according to any one of the preceding embodiments, wherein the nanopore is a protein pore, a solid state pore or a DNA origami pore, preferably wherein the nanopore is a transmembrane protein pore.
20. A method according to any one of the preceding embodiments, wherein the one or more characteristics comprises (i) the length of the polynucleotide, (ii) the identity of the polynucleotide, (iii) the sequence of the polynucleotide, (iv) the secondary structure of the polynucleotide and (v) whether or not the polynucleotide is modified.
21. A method according to any one of the embodiments 4 to 20, wherein ejecting the polynucleotide from the nanopore comprises applying a force across the nanopore.
22. A method according to embodiment 20, wherein the force comprises a voltage potential applied across the nanopore.
23. A method according to any one of the preceding embodiments, comprising applying a voltage potential across the nanopore via one or more platinum electrodes.
24. A kit for characterising a polynucleotide, comprising:
   (a) a nanopore in a membrane; and
   (b) a solution comprising a sodium ferricyanide/sodium ferrocyanide salt.
25 An apparatus for characterising a polynucleotide, comprising:
   (a) an array of nanopores in a membrane; and
   (b) a sodium ferricyanide/sodium ferrocyanide salt solution in contact with the membrane.
26. Use of a sodium salt for reducing cation-mediated secondary structure in a polynucleotide in a nanopore apparatus or during a method of characterising a polynucleotide using a nanopore.

SEQUENCE LISTING
exonuclease I from E. coli
SEQ ID NO: 1
MMNDGKQQSTFLFHDYETFGTHPALDRPAQFAAIRTDSEFNVIGEPEVFYCKPADDYLPQ
PGAVLITGITPQEARAKGENEAAFAARIHSLFTVPKTCILGYNNVREDDEVTRNIFYRNF
YDPYAWSWQHDNSRWDLLDVMRACYALRPEGINWPENDDGLPSFRLEHLTKANGIEHSNA
HDAMADVYATIAMAKLVKTRQPRLFDYLFTHRNKHKLMALIDVPQMKPLVHVSGMFGAWR
GNTSWVAPLAWHPENRNAVIMVDLAGDISPLLELDSDTLRERLYTAKTDLGDNAAVPVKL
VHINKCPVLAQANTLRPEDADRLGINRQHCLDNLKILRENPQVREKVVAIFAEAEPFTPS
DNVDAQLYNGFFSDADRAAMKIVLETEPRNLPALDITFVDKRIEKLLFNYRARNFPGTLD
YAEQQRWLEHRRQVFTPEFLQGYADELQMLVQQYADDKEKVALLKALWQYAEEIVSGSGH
HHHHH
exonuclease III enzyme from E. coli
SEQ ID NO: 2
MKFVSFNINGLRARPHQLEAIVEKHQPDVIGLQETKVHDDMFPLEEVAKLGYNVFYHGQK
GHYGVALLTKETPIAVRRGFPGDDEEAQRRIIMAEIPSLLGNVTVINGYFPQGESRDHPI
KFPAKAQFYQNLQNYLETELKRDNPVLIMGDMNISPTDLDIGIGEENRKRWLRTGKCSFL
PEEREWMDRLMSWGLVDTFRHANPQTADRFSWFDYRSKGFDDNRGLRIDLLLASQPLAEC
CVETGIDYEIRSMEKPSDHAPVWATFRR
RecJ enzyme from T. thermophilus
SEQ ID NO: 3
MFRRKEDLDPPLALLPLKGLREAAALLEEALRQGKRIRVHGDYDADGLTGTAILVRGLAA
LGADVHPFIPHRLEEGYGVLMERVPEHLEASDLFLTVDCGITNHAELRELLENGVEVIVT
DHHTPGKTPPPGLVVHPALTPDLKEKPTGAGVAFLLLWALHERLGLPPPLEYADLAAVGT
IADVAPLWGWNRALVKEGLARIPASSWVGLRLLAEAVGYTGKAVEVAFRIAPRINAASRL
GEAEKALRLLLTDDAAEAQALVGELHRLNARRQTLEEAMLRKLLPQADPEAKAIVLLDPE
GHPGVMGIVASRILEATLRPVFLVAQGKGTVRSLAPISAVEALRSAEDLLLRYGGHKEAA
GFAMDEALFPAFKARVEAYAARFPDPVREVALLDLLPEPGLLPQVFRELALLEPYGEGNP
EPLFL
bacteriophage lambda exonuclease
SEQ ID NO: 4
MTPDIILQRTGIDVRAVEQGDDAWHKLRLGVITASEVHNVIAKPRSGKKWPDMKMSYFHT
LLAEVCTGVAPEVNAKALAWGKQYENDARTLFEFTSGVNVTESPIIYRDESMRTACSPDG
LCSDGNGLELKCPFTSRDFMKFRLGGFEAIKSAYMAQVQYSMWVTRKNAWYFANYDPRMK
REGLHYVVIERDEKYMASFDEIVPEFIEKMDEALAEIGFVFGEQWR
Phi29 DNA polymerase
SEQ ID NO: 5
MKHMPRKMYSCAFETTTKVEDCRVWAYGYMNIEDHSEYKIGNSLDEFMAWVLKVQADLYF
HNLKFDGAFIINWLERNGFKWSADGLPNTYNTIISRMGQWYMIDICLGYKGKRKIHTVIY
DSLKKLPFPVKKIAKDFKLTVLKGDIDYHKERPVGYKITPEEYAYIKNDIQIIAEALLIQ
FKQGLDRMTAGSDSLKGFKDIITTKKFKKVFPTLSLGLDKEVRYAYRGGFTWLNDRFKEK
EIGEGMVFDVNSLYPAQMYSRLLPYGEPIVFEGKYVWDEDYPLHIQHIRCEFELKEGYIP
TIQIKRSRFYKGNEYLKSSGGEIADLWLSNVDLELMKEHYDLYNVEYISGLKFKATTGLF
KDFIDKWTYIKTTSEGAIKQLAKLMLNSLYGKFASNPDVTGKVPYLKENGALGFRLGEEE
TKDPVYTPMGVFITAWARYTTITAAQACYDRIIYCDTDSIHLTGTEIPDVIKDIVDPKKL
GYWAHESTFKRAKYLRQKTYIQDIYMKEVDGKLVEGSPDDYTDIKFSVKCAGMTDKIKKE
VTFENFKVGFSRKMKPKPVQVPGGVVLVDDTFTIKSGGSAWSHPQFEKGGGSGGGSGGSA
WSHPQFEK
Trwc Cba helicase
SEQ ID NO: 6
MLSVANVRSPSAAASYFASDNYYASADADRSGQWIGDGAKRLGLEGKVEARAFDALLRGE
LPDGSSVGNPGQAHRPGTDLTFSVPKSWSLLALVGKDERIIAAYREAVVEALHWAEKNAA
ETRVVEKGMVVTQATGNLAIGLFQHDTNRNQEPNLHFHAVIANVTQGKDGKWRTLKNDRL
WQLNTTLNSIAMARFRVAVEKLGYEPGPVLKHGNFEARGISREQVMAFSTRRKEVLEARR
GPGLDAGRIAALDTRASKEGIEDRATLSKQWSEAAQSIGLDLKPLVDRARTKALGQGMEA
TRIGSLVERGRAWLSRFAAHVRGDPADPLVPPSVLKQDRQTIAAAQAVASAVRHLSQREA
AFERTALYKAALDFGLPTTIADVEKRTRALVRSGDLIAGKGEHKGWLASRDAVVTEQRIL
SEVAAGKGDSSPAITPQKAAASVQAAALTGQGFRLNEGQLAAARLILISKDRTIAVQGIA
GAGKSSVLKPVAEVLRDEGHPVIGLAIQNTLVQMLERDTGIGSQTLARFLGGWNKLLDDP
GNVALRAEAQASLKDHVLVLDEASMVSNEDKEKLVRLANLAGVHRLVLIGDRKQLGAVDA
GKPFALLQRAGIARAEMATNLRARDPVVREAQAAAQAGDVRKALRHLKSHTVEARGDGAQ
VAAETWLALDKETRARTSIYASGRAIRSAVNAAVQQGLLASREIGPAKMKLEVLDRVNTT
REELRHLPAYRAGRVLEVSRKQQALGLFIGEYRVIGQDRKGKLVEVEDKRGKRFRFDPAR
IRAGKGDDNLTLLEPRKLEIHEGDRIRWTRNDHRRGLFNADQARVVEIANGKVTFETSKG
DLVELKKDDPMLKRIDLAYALNVHMAQGLTSDRGIAVMDSRERNLSNQKTFLVTVTRLRD
HLTLVVDSADKLGAAVARNKGEKASAIEVTGSVKPTATKGSGVDQPKSVEANKAEKELTR
SKSKTLDFGI
Hel308 Mou helicase
SEQ ID NO: 7
MMIRELDIPRDIIGFYEDSGIKELYPPQAEAIEMGLLEKKNLLAAIPTASGKTLLAELAM
IKAIREGGKALYIVPLRALASEKFERFKELAPFGIKVGISTGDLDSRADWLGVNDIIVAT
SEKTDSLLRNGTSWMDEITTVVVDEIHLLDSKNRGPTLEVTITKLMRLNPDVQVVALSAT
VGNAREMADWLGAALVLSEWRPTDLHEGVLFGDAINFPGSQKKIDRLEKDDAVNLVLDTI
KAEGQCLVFESSRRNCAGFAKTASSKVAKILDNDIMIKLAGIAEEVESTGETDTAIVLAN
CIRKGVAFHHAGLNSNHRKLVENGFRQNLIKVISSTPTLAAGLNLPARRVIIRSYRREDS
NFGMQPIPVLEYKQMAGRAGRPHLDPYGESVLLAKTYDEFAQLMENYVEADAEDIWSKLG
TENALRTHVLSTIVNGFASTRQELFDFFGATFFAYQQDKWMLEEVINDCLEFLIDKAMVS
ETEDIEDASKLFLRGTRLGSLVSMLYIDPLSGSKIVDGFKDIGKSTGGNMGSLEDDKGDD
ITVTDMTLLHLVCSTPDMRQLYLRNTDYTIVNEYIVAHSDEFHEIPDKLKETDYEWFMGE
VKTAMLLEEWVTEVSAEDITRHFNVGEGDIHALADTSEWLMHAAAKLAELLGVEYSSHAY
SLEKRIRYGSGLDLMELVGIRGVGRVRARKLYNAGFVSVAKLKGADISVLSKLVGPKVAY
NILSGIGVRVNDKHENSAPISSNTLDTLLDKNQKTENDFQ
Dda helicase
SEQ ID NO: 8
MTFDDLTEGQKNAFNIVMKAIKEKKHHVTINGPAGTGKTTLTKFIIEALISTGETGIILA
APTHAAKKILSKLSGKEASTIHSILKINPVTYEENVLFEQKEVPDLAKCRVLICDEVSMY
DRKLFKILLSTIPPWCTIIGIGDNKQIRPVDPGENTAYISPFFTHKDFYQCELTEVKRSN
APIIDVATDVRNGKWIYDKVVDGHGVRGFTGDTALRDFMVNYFSIVKSLDDLFENRVMAF
TNKSVDKLNSIIRKKIFETDKDFIVGEIIVMQEPLFKTYKIDGKPVSEIIFNNGQLVRII
EAEYTSTFVKARGVPGEYLIRHWDLTVETYGDDEYYREKIKIISSDEELYKFNLFLGKTA
ETYKNWNKGGKAPWSDFWDAKSQFSKVKALPASTFHKAQGMSVDRAFIYTPCIHYADVEL
AQQLLYVGVTRGRYDVFYV

1. A method of characterising a polynucleotide, comprising:
(a) contacting the polynucleotide with a nanopore having a first opening and a second opening;
(b) translocating a portion of the polynucleotide through the nanopore in a first direction with respect to the nanopore, wherein the first direction is from the first opening to the second opening;
(c) contacting the translocated portion of the polynucleotide with one or more conditions which decrease formation of cation-mediated secondary structure;
(d) translocating the polynucleotide in a second direction with respect to the nanopore, wherein the second direction is from the second opening to the first opening; and
(e) taking one or more measurements characteristic of the polynucleotide during the translocation of the polynucleotide through the nanopore, thereby characterising the polynucleotide.

2. A method according to claim 1, wherein if the measurements characteristic of the polynucleotide are indicative of one or more desired characteristics of the polynucleotide, the method comprises translocating the entire polynucleotide through the nanopore and taking one or more measurements during said translocation.

3. A method according to claim 1 or claim 2, comprising repeating steps (b) to (d) multiple times.

4. A method according to claim 1, wherein if the measurements characteristic of the polynucleotide are indicative of one or more undesired characteristics of the polynucleotide, the method comprises ejecting the polynucleotide from the first opening of the nanopore.

5. A method according to claim 4, wherein the one or more undesired characteristics include the inability of the polynucleotide to fully translocate through the nanopore in the first direction with respect to the nanopore.

6. A method according to any one of the preceding claims, wherein the method comprises characterising a plurality of polynucleotides using a plurality of nanopores, optionally wherein said plurality of nanopores are present in a nanopore array.

7. A method of operating a nanopore array, wherein each nanopore in the array has a first opening and a second opening, the method comprising:
(a) contacting the array with a plurality of polynucleotides under conditions such that a portion of each polynucleotide translocates through the array in a first direction with respect to the nanopores in the array, wherein the first direction is from the first opening to the second opening;
(b) contacting the translocated portion of the polynucleotides with one or more conditions which decrease formation of cation-mediated secondary structure;
(c) determining whether each polynucleotide translocated in step (b) has a desired characteristic; and
(d) ejecting polynucleotides which do not have the desired characteristic by translocating the polynucleotide in a second direction with respect to the nanopores in the array, wherein the second direction is from the second opening to the first opening.

8. A method according to claim 7, comprising taking one or more measurements characteristic of the polynucleotides in the plurality of polynucleotides during the translocation of the portion of each polynucleotide through the nanopore, thereby characterising the polynucleotides.

9. A method according to any one of the preceding claims, wherein the method is performed at a temperature lower than the melting temperature of the cation-mediated secondary structure.

10. A method according to any one of the preceding claims, wherein the cation-mediated secondary structure is a guanine tetrad or a G-quadruplex, preferably a G-quadruplex.

11. A method according to any one of the preceding claims, wherein the one or more conditions comprise a metal salt that disrupts the stability of the cation-mediated secondary structure.

12. A method according to claim 11, wherein the metal salt comprises a cation selected from the group consisting of Rb⁺, Na⁺, Cs⁺, Li⁺, Mg²⁺ and Ca²⁺.

13. A method according to claim 11 or 12, wherein the metal salt comprises a Rb⁺ cation.

14. A method according to any one of claims 11 to 13, wherein the metal salt comprises (i) rubidium ferricyanide and/or rubidium ferrocyanide, and/or (ii) rubidium chloride.

15. A method according to claim 11 or 12, wherein the metal salt comprises a Na⁺ cation.

16. A method according to any one of claims 11, 12 and 15, wherein the metal salt comprises sodium ferricyanide and/or sodium ferrocyanide.

17. A method according to any one of the preceding claims, wherein the conditions are substantially free of K⁺.

18. A method according to any one of the preceding claims, wherein the polynucleotide, or each polynucleotide in the plurality of the polynucleotides, has a length of 100 nucleotides or more.

19. A method according to any one of the preceding claims, wherein the polynucleotide has a length of 5000 nucleotides or more, or the polynucleotides in the plurality of polynucleotides have an average length of 5000 nucleotides or more.

20. A method according to any one of the preceding claims, comprising controlling the movement of the or each polynucleotide with respect to the nanopore in the first direction using a polynucleotide-handling protein.

21. A method according to claim 20, wherein the polynucleotide-handling protein is or comprises a helicase, translocase or helicase-nuclease complex.

22. A method according to any one of the preceding claims, wherein the nanopore is a protein pore, a solid state pore or a DNA origami pore, preferably wherein the nanopore is a transmembrane protein pore.

23. A method according to any one of the preceding claims, wherein the one or more characteristics comprises (i) the length of the polynucleotide, (ii) the identity of the polynucleotide, (iii) the sequence of the polynucleotide, (iv) the secondary structure of the polynucleotide and (v) whether or not the polynucleotide is modified.

24. A method according to any one of claims 4 to 23, wherein ejecting the polynucleotide from the nanopore comprises applying a force across the nanopore.

25. A method according to claim 21, wherein the force comprises a voltage potential applied across the nanopore.

26. A method according to any one of the preceding claims, comprising applying a voltage potential across the nanopore via one or more platinum electrodes.

27. A kit for characterising a polynucleotide, comprising:
(a) a nanopore in a membrane; and
(b) a solution comprising one or more metal salts selected from the group consisting of one or more rubidium salts, sodium ferricyanide and sodium ferrocyanide.

28. A kit according to claim 27, wherein the one or more metal salts are selected from the group consisting of (i) rubidium ferricyanide and/or rubidium ferrocyanide, (ii) rubidium chloride, and/or (iii) sodium ferricyanide and sodium ferrocyanide.

29. An apparatus for characterising a polynucleotide, comprising:
(a) an array of nanopores in a membrane; and
(b) a solution comprising one or more metal salts in contact with the membrane, wherein the one or more metal salts are selected from the group consisting of one or more rubidium salts, sodium ferricyanide and sodium ferrocyanide.

30. An apparatus according to claim 29, wherein the one or more metal salts are selected from the group consisting of (i) rubidium ferricyanide and/or rubidium ferrocyanide, (ii) rubidium chloride, and (iii) sodium ferricyanide and sodium ferrocyanide.

31. An apparatus according to claim 29 or 30, wherein the array is an array of individually addressable elements, wherein each independently addressable element of the array comprises a cis and a trans chamber connected by an electrical communication means, wherein said cis and trans chambers are separated by the membrane, and the membrane of each independently addressable element comprises a single nanopore.

32. An apparatus according to claim 31, wherein:
(i) the cis and the trans chamber each comprise the solution; or
(ii) the trans chamber comprises the solution, and the cis chamber comprises a different solution comprising one or more metal salts.

33. An apparatus according to claim 31 or 32, wherein:
(i) the apparatus comprises a polynucleotide handling enzyme on the cis side of the membrane;
(ii) the independently addressable elements of the array share a common cis chamber;
(iii) each independently addressable element has a trans chamber separate from the trans chambers of other independently addressable elements of the array;
(iv) the apparatus comprises a voltage source connected to electrodes, wherein the voltage source and electrodes are configured to apply an electric field between the cis and the trans chambers; and/or
(v) the apparatus comprises a digital logic circuit associated with the nanopores of the independently addressable elements of the array of independently addressable elements.

34. Use of a one or more metal salts for reducing cation-mediated secondary structure in a polynucleotide in a nanopore apparatus or during a method of characterising a polynucleotide using a nanopore, wherein the one or more metal salts are selected from the group consisting of one or more rubidium salts, sodium ferricyanide and sodium ferrocyanide.

35. A use according to claim 34, wherein the one or more metal salts are selected from the group consisting of (i) rubidium ferricyanide and/or rubidium ferrocyanide, (ii) rubidium chloride, and (iii) sodium ferricyanide and sodium ferrocyanide.


SEQUENCE LISTING (9 sequences)

SEQ ID NO: 1 - protein, 485 aa, Escherichia coli
MMNDGKQQSTFLFHDYETFGTHPALDRPAQFAAIRTDSEFNVIGEPEVFYCKPADDYLPQPGAVLITGITPQEARAKGENEAAFAARIHSLFTVPKTCILGYNNVRFDDEVTRNIFYRNFYDPYAWSWQHDNSRWDLLDVMRACYALRPEGINWPENDDGLPSFRLEHLTKANGIEHSNAHDAMADVYATIAMAKLVKTRQPRLFDYLFTHRNKHKLMALIDVPQMKPLVHVSGMFGAWRGNTSWVAPLAWHPENRNAVIMVDLAGDISPLLELDSDTLRERLYTAKTDLGDNAAVPVKLVHINKCPVLAQANTLRPEDADRLGINRQHCLDNLKILRENPQVREKVVAIFAEAEPFTPSDNVDAQLYNGFFSDADRAAMKIVLETEPRNLPALDITFVDKRIEKLLFNYRARNFPGTLDYAEQQRWLEHRRQVFTPEFLQGYADELQMLVQQYADDKEKVALLKALWQYAEEIVSGSGHHHHHH

SEQ ID NO: 2 - protein, 268 aa, Escherichia coli
MKFVSFNINGLRARPHQLEAIVEKHQPDVIGLQETKVHDDMFPLEEVAKLGYNVFYHGQKGHYGVALLTKETPIAVRRGFPGDDEEAQRRIIMAEIPSLLGNVTVINGYFPQGESRDHPIKFPAKAQFYQNLQNYLETELKRDNPVLIMGDMNISPTDLDIGIGEENRKRWLRTGKCSFLPEEREWMDRLMSWGLVDTFRHANPQTADRFSWFDYRSKGFDDNRGLRIDLLLASQPLAECCVETGIDYEIRSMEKPSDHAPVWATFRR

SEQ ID NO: 3 - protein, 425 aa, Thermus thermophilus
MFRRKEDLDPPLALLPLKGLREAAALLEEALRQGKRIRVHGDYDADGLTGTAILVRGLAALGADVHPFIPHRLEEGYGVLMERVPEHLEASDLFLTVDCGITNHAELRELLENGVEVIVTDHHTPGKTPPPGLVVHPALTPDLKEKPTGAGVAFLLLWALHERLGLPPPLEYADLAAVGTIADVAPLWGWNRALVKEGLARIPASSWVGLRLLAEAVGYTGKAVEVAFRIAPRINAASRLGEAEKALRLLLTDDAAEAQALVGELHRLNARRQTLEEAMLRKLLPQADPEAKAIVLLDPEGHPGVMGIVASRILEATLRPVFLVAQGKGTVRSLAPISAVEALRSAEDLLLRYGGHKEAAGFAMDEALFPAFKARVEAYAARFPDPVREVALLDLLPEPGLLPQVFRELALLEPYGEGNPEPLFL

SEQ ID NO: 4 - protein, 226 aa, Escherichia virus Lambda
MTPDIILQRTGIDVRAVEQGDDAWHKLRLGVITASEVHNVIAKPRSGKKWPDMKMSYFHTLLAEVCTGVAPEVNAKALAWGKQYENDARTLFEFTSGVNVTESPIIYRDESMRTACSPDGLCSDGNGLELKCPFTSRDFMKFRLGGFEAIKSAYMAQVQYSMWVTRKNAWYFANYDPRMKREGLHYVVIERDEKYMASFDEIVPEFIEKMDEALAEIGFVFGEQWR

SEQ ID NO: 5 - protein, 608 aa, Bacillus subtilis phage Phi29
MKHMPRKMYSCAFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKSGGSAWSHPQFEKGGGSGGGSGGSAWSHPQFEK

SEQ ID NO: 6 - protein, 970 aa, Citromicrobium bathyomarinum
MLSVANVRSPSAAASYFASDNYYASADADRSGQWIGDGAKRLGLEGKVEARAFDALLRGELPDGSSVGNPGQAHRPGTDLTFSVPKSWSLLALVGKDERIIAAYREAVVEALHWAEKNAAETRVVEKGMVVTQATGNLAIGLFQHDTNRNQEPNLHFHAVIANVTQGKDGKWRTLKNDRLWQLNTTLNSIAMARFRVAVEKLGYEPGPVLKHGNFEARGISREQVMAFSTRRKEVLEARRGPGLDAGRIAALDTRASKEGIEDRATLSKQWSEAAQSIGLDLKPLVDRARTKALGQGMEATRIGSLVERGRAWLSRFAAHVRGDPADPLVPPSVLKQDRQTIAAAQAVASAVRHLSQREAAFERTALYKAALDFGLPTTIADVEKRTRALVRSGDLIAGKGEHKGWLASRDAVVTEQRILSEVAAGKGDSSPAITPQKAAASVQAAALTGQGFRLNEGQLAAARLILISKDRTIAVQGIAGAGKSSVLKPVAEVLRDEGHPVIGLAIQNTLVQMLERDTGIGSQTLARFLGGWNKLLDDPGNVALRAEAQASLKDHVLVLDEASMVSNEDKEKLVRLANLAGVHRLVLIGDRKQLGAVDAGKPFALLQRAGIARAEMATNLRARDPVVREAQAAAQAGDVRKALRHLKSHTVEARGDGAQVAAETWLALDKETRARTSIYASGRAIRSAVNAAVQQGLLASREIGPAKMKLEVLDRVNTTREELRHLPAYRAGRVLEVSRKQQALGLFIGEYRVIGQDRKGKLVEVEDKRGKRFRFDPARIRAGKGDDNLTLLEPRKLEIHEGDRIRWTRNDHRRGLFNADQARVVEIANGKVTFETSKGDLVELKKDDPMLKRIDLAYALNVHMAQGLTSDRGIAVMDSRERNLSNQKTFLVTVTRLRDHLTLVVDSADKLGAAVARNKGEKASAIEVTGSVKPTATKGSGVDQPKSVEANKAEKELTRSKSKTLDFGI

SEQ ID NO: 7 - protein, 760 aa, Methanococcoides burtonii
MMIRELDIPRDIIGFYEDSGIKELYPPQAEAIEMGLLEKKNLLAAIPTASGKTLLAELAMIKAIREGGKALYIVPLRALASEKFERFKELAPFGIKVGISTGDLDSRADWLGVNDIIVATSEKTDSLLRNGTSWMDEITTVVVDEIHLLDSKNRGPTLEVTITKLMRLNPDVQVVALSATVGNAREMADWLGAALVLSEWRPTDLHEGVLFGDAINFPGSQKKIDRLEKDDAVNLVLDTIKAEGQCLVFESSRRNCAGFAKTASSKVAKILDNDIMIKLAGIAEEVESTGETDTAIVLANCIRKGVAFHHAGLNSNHRKLVENGFRQNLIKVISSTPTLAAGLNLPARRVIIRSYRRFDSNFGMQPIPVLEYKQMAGRAGRPHLDPYGESVLLAKTYDEFAQLMENYVEADAEDIWSKLGTENALRTHVLSTIVNGFASTRQELFDFFGATFFAYQQDKWMLEEVINDCLEFLIDKAMVSETEDIEDASKLFLRGTRLGSLVSMLYIDPLSGSKIVDGFKDIGKSTGGNMGSLEDDKGDDITVTDMTLLHLVCSTPDMRQLYLRNTDYTIVNEYIVAHSDEFHEIPDKLKETDYEWFMGEVKTAMLLEEWVTEVSAEDITRHFNVGEGDIHALADTSEWLMHAAAKLAELLGVEYSSHAYSLEKRIRYGSGLDLMELVGIRGVGRVRARKLYNAGFVSVAKLKGADISVLSKLVGPKVAYNILSGIGVRVNDKHFNSAPISSNTLDTLLDKNQKTFNDFQ

SEQ ID NO: 8 - protein, 439 aa, Escherichia virus T4
MTFDDLTEGQKNAFNIVMKAIKEKKHHVTINGPAGTGKTTLTKFIIEALISTGETGIILAAPTHAAKKILSKLSGKEASTIHSILKINPVTYEENVLFEQKEVPDLAKCRVLICDEVSMYDRKLFKILLSTIPPWCTIIGIGDNKQIRPVDPGENTAYISPFFTHKDFYQCELTEVKRSNAPIIDVATDVRNGKWIYDKVVDGHGVRGFTGDTALRDFMVNYFSIVKSLDDLFENRVMAFTNKSVDKLNSIIRKKIFETDKDFIVGEIIVMQEPLFKTYKIDGKPVSEIIFNNGQLVRIIEAEYTSTFVKARGVPGEYLIRHWDLTVETYGDDEYYREKIKIISSDEELYKFNLFLGKTAETYKNWNKGGKAPWSDFWDAKSQFSKVKALPASTFHKAQGMSVDRAFIYTPCIHYADVELAQQLLYVGVTRGRYDVFYV

SEQ ID NO: 9 - DNA, 15 bp, synthetic construct
  source 1..15: mol_type = other DNA
ggttggtgtggttgg